(19) United States
(12) Patent Application Publication
(10) Pub. No.: US 2023/0242974 A1
(43) Pub. Date: Aug. 3, 2023

(54) METHODS AND COMPOSITIONS FOR ROLLING CIRCLE AMPLIFICATION

(71) Applicant: 10x Genomics, Inc., Pleasanton, CA (US)

(72) Inventors: Justin COSTA, Union City, CA (US); Jorge Iván HERNÁNDEZ NEUTA, Stockholm (SE)

(73) Assignee: 10x Genomics, Inc., Pleasanton, CA (US)

(21) Appl. No.: 18/088,330

(22) Filed: Dec. 23, 2022

Related U.S. Application Data
(60) Provisional application No. 63/320,645, filed on Mar. 16, 2022.
(60) Provisional application No. 63/294,037, filed on Dec. 27, 2021.

(51) Int. Cl. C12Q 1/6841; C12Q 1/6855

(52) U.S. Cl. CPC C12Q 1/6841; C12Q 1/6855

(57) ABSTRACT

The present disclosure in some aspects relates to methods and compositions for detecting and quantifying multiple analytes present in a biological sample by rolling circle amplification (RCA). In some aspects, the methods and compositions provided herein address issues associated with control of signal spot size and intensity for RCA products (RCPs) in a sample. In some aspects, provided herein are methods and compositions (e.g., probes and/or reaction mixtures) for slowing down RCA and/or decreasing the size of RCA products. In some embodiments, nucleotides or nucleotide analogs having a hydrophobic group (e.g., a hydrophobic modification on the base) are included in nucleotides for RCA and incorporated into the RCP product, leading to reduction of RCP size without the need of crosslinking an incorporated hydrophobic nucleotide or nucleotide analog to the RCP itself or another molecule.

### CROSS-REFERENCE TO RELATED APPLICATIONS

This application claims priority to U.S. Provisional Patent Application No. 63/294,037, filed Dec. 27, 2021, entitled “METHODS AND COMPOSITIONS FOR SLOWING ROLLING CIRCLE AMPLIFICATION,” and to U.S. Provisional Patent Application No. 63/320,645, filed Mar. 16, 2022, entitled “METHODS AND COMPOSITIONS FOR ROLLING CIRCLE AMPLIFICATION SLOWING OR COMPACTION,” each of which is herein incorporated by reference in its entirety for all purposes.

### FIELD

The present disclosure generally relates to methods and compositions for rolling circle amplification (RCA), e.g., in situ detection of analytes by RCA.

### BACKGROUND

Genomic, transcriptomic, and proteomic profiling of cells and tissue samples using microscopic imaging can resolve multiple analytes of interest at the same time, thereby providing valuable information regarding analyte abundance and localization in situ. Thus, in situ assays (e.g., rolling circle amplification (RCA)-based methods) are important tools, for example, for understanding the molecular basis of cell identity and developing treatment for diseases. The size of detected RCA products, however, may limit the ability to resolve individual RCA products corresponding to target analytes. Large signal spots may overlap with one another and/or mask adjacent smaller signal spots, rendering the signal spots unresolvable. In addition, some analytes may be associated with bright signal spots (e.g., due to high analyte abundance and/or preferential signal amplification), while other analytes may be associated with signal spots that are too dim to be detected simultaneously with the bright spots. There is a need for new and improved methods for in situ assays. The present disclosure addresses these and other needs.

### SUMMARY

In some aspects, provided herein is a method for analyzing a biological sample, comprising: (a) contacting the biological sample with a reaction mixture comprising one or more modified nucleotides or nucleotide analogs comprising a modified nucleotide or nucleotide analog having a hydrophobic modification, (b) performing rolling circle amplification (RCA) of a circular nucleic acid template in the biological sample using a polymerase, thereby generating an RCA product incorporating the one or more modified nucleotides or nucleotide analogs, wherein the RCA product is not crosslinked via the one or more modified nucleotides or nucleotide analogs incorporated into the RCA product, and. (c) detecting the RCA product not crosslinked via the one or more modified nucleotides or nucleotide analogs at a location in the biological sample.

In some embodiments, the RCA product is not crosslinked to another molecule via the one or more modified nucleotides or nucleotide analogs incorporated into the RCA product. In some embodiments, the RCA product is not crosslinked, via the one or more modified nucleotides or nucleotide analogs incorporated into the RCA product, to itself, to another molecule in the biological sample, or to a matrix embedding the biological sample.

In some embodiments, the hydrophobic modification is a base modification. In some embodiments, the hydrophobic modification comprises a carbon chain and/or a hydrocarbon ring. In some embodiments, the hydrophobic modification comprises a triple bond. In some embodiments, the hydrophobic modification comprises a vinyl or ethynyl group. In some embodiments, the modified nucleotide or nucleotide analog comprising the hydrophobic modification is an ethynyl-dUTP or a vinyl-dUTP. In some embodiments, the modified nucleotide or nucleotide analog comprising the hydrophobic modification is a 5-ethynyl-dUTP or a 5-vinyl-dUTP.

In some embodiments, the diameter of the RCA product generated using the one or more modified nucleotides or nucleotide analogs is smaller than a reference RCA product produced using the same template without including the one or more modified nucleotides or nucleotide analogs in the reaction mixture.

In some embodiments, the modified nucleotide or nucleotide analog comprising the hydrophobic modification is added to the biological sample at a concentration of at least 1 μM, at least 1.25 μM, at least 2.5 μM, at least 5 μM, at least 10 μM, at least 40 μM, at least 80 μM, or at least 100 μM. In some embodiments, the modified nucleotide or nucleotide analog is a modified dUTP and the ratio of the modified dUTP to an unmodified dUTP or dTTP in the reaction mixture is between about 80:20 and about 1:99. In some embodiments, the ratio of the modified dUTP to the unmodified dUTP or dTTP in the reaction mixture is between about 80:20 and about 40:60. In some embodiments, the modified nucleotide or nucleotide analog comprising the hydrophobic modification is added to the biological sample at a concentration of about 50 μM to about 100 μM. In some embodiments, the modified nucleotide or nucleotide analog comprising the hydrophobic modification is added to the biological sample at a concentration of about 80 μM to about 100 μM.

In some embodiments, the median diameter of an RCA product generated using the one or more modified nucleotides or nucleotide analogs is smaller than the median diameter of a reference RCA product produced using the same template without including the one or more modified nucleotides or nucleotide analogs in the reaction mixture. In some embodiments, the median diameter of an RCA product generated using the one or more modified nucleotides or nucleotide analogs is less than 500 nm. In some embodiments, the median diameter of an RCA product generated using the one or more modified nucleotides or nucleotide analogs is no more than 90% or no more than 80% of the median diameter of a reference RCA product produced using the same template without including the one or more modified nucleotides or nucleotide analogs in the reaction mixture.

In some embodiments, the average intensity, average signal-to-noise ratio, and/or density of an RCA product incorporating the one or more modified nucleotides or nucleotide analogs is not significantly different from the average intensity, average signal-to-noise ratio, and/or density of a reference RCA product produced using the same template without including the one or more modified nucleotides or nucleotide analogs in the reaction mixture.

In some embodiments, the incorporation of the one or more modified nucleotides or nucleotide analogs into the RCA product increases overall hydrophobicity of the RCA product. In some embodiments, the incorporation of the one or more modified nucleotides or nucleotide analogs into the RCA product promotes base stacking interactions among nucleotides in the RCA product.

In some embodiments, the one or more modified nucleotides or nucleotide analogs incorporated into the RCA product do not comprise a detectable label. In some embodiments, the detectable label is a fluorophore. In some embodiments, detecting the RCA product comprises contacting the biological sample with a nucleic acid probe that hybridizes to the RCA product. In some embodiments, the nucleic acid probe comprises a detectable label. In some embodiments, the detectable label is a fluorophore. In some embodiments, the nucleic acid probe is an intermediate probe, and the method comprises contacting the biological sample with a detectably labeled probe that hybridizes to the intermediate probe.

In some aspects, provided herein is a method for analyzing a biological sample, comprising: (a) contacting the biological sample with a reaction mixture comprising one or more modified nucleotides or nucleotide analogs, (b) performing rolling circle amplification (RCA) of a circular nucleic acid template in the biological sample using a polymerase, thereby generating an RCA product, wherein the one or more modified nucleotides or nucleotide analogs comprise: (i) a non-incorporable nucleotide or analog thereof that is not incorporated by the polymerase, and/or (ii) an incorporable modified nucleotide or nucleotide analog that is configured to be incorporated by the polymerase at a slower rate than a corresponding nucleoside triphosphate, and (c) detecting the RCA product which is not crosslinked via modified nucleotide(s) or nucleotide analog(s) at a location in the biological sample.

In some embodiments, the RCA product is not crosslinked, via any modified nucleotide or nucleotide analog incorporated into the RCA product, to the RCA product itself, to another molecule in the biological sample, or to a matrix embedding the biological sample. In some embodiments, the one or more modified nucleotides or nucleotide analogs incorporated into the RCA product do not comprise a detectable label. In some embodiments, the detectable label is a fluorophore.

In some embodiments, detecting the RCA product comprises contacting the biological sample with a nucleic acid probe that hybridizes to the RCA product. In some embodiments, the nucleic acid probe comprises a detectable label. In some embodiments, the detectable label is a fluorophore. In some embodiments, the nucleic acid probe is an intermediate probe, and the method comprises contacting the biological sample with a detectably labeled probe that hybridizes to the intermediate probe

In some embodiments, the presence of the one or more modified nucleotides or nucleotide analogs in the reaction mixture decreases the polymerization rate of the polymerase and/or the size of the RCA product as compared to a reference reaction mixture without the one or more modified nucleotides or nucleotide analogs. In some embodiments, the reference reaction mixture comprises only unmodified dATP, dTTP and/or dUTP, dCTP, and dGTP.

In some aspects, provided herein is a method for analyzing a biological sample, comprising: (a) contacting the biological sample with a circular probe or circularizable probe or probe set comprising one or more modified nucleotide or nucleotide analog residues, wherein the circular probe or circularizable probe or probe set comprises a hybridization region that hybridizes to a target nucleic acid in the biological sample, and wherein the one or more modified nucleotide or nucleotide analog residues are outside the hybridization region, and (b) using a polymerase to perform rolling circle amplification (RCA) of the circular probe or of a circularized probe generated from the circularizable probe or probe set, thereby generating an RCA product, wherein the presence of the one or more modified nucleotide or nucleotide analog residues decreases the polymerization rate of the polymerase on the circular or circularized probe and/or the size of the RCA product as compared to a reference circular template without the one or more modified nucleotide or nucleotide analog residues.

In some embodiments, the one or more modified nucleotide or nucleotide analog residues comprise modified deoxyribonucleotide (DNA) or DNA analog residues. In some embodiments, the one or more modified nucleotide or nucleotide analog residues comprise modified ribonucleotide (RNA) or RNA analog residues. In some embodiments, the method comprises detecting the RCA product at a location in the biological sample.

In some aspects, provided herein is a method for analyzing a biological sample, comprising: (a) contacting the biological sample with a circular probe or circularizable probe or probe set comprising one or more modified nucleotide or nucleotide analog residues, wherein the circular probe or circularizable probe or probe set hybridizes to a target nucleic acid in the biological sample, and (b) using a polymerase to perform rolling circle amplification (RCA) of the circular probe or of a circularized probe generated from the circularizable probe or probe set, thereby generating an RCA product, wherein the presence of the one or more modified nucleotide or nucleotide analog residues decreases the polymerization rate of the polymerase on the circular or circularized probe as compared to a reference circular template without the one or more modified nucleotide or nucleotide analog residues. In some embodiments, the one or more modified nucleotide or nucleotide analog residues can comprise a sugar modified nucleotide. In any of the preceding embodiments, the one or more modified nucleotide or nucleotide analog residues can comprise a C2′ modified nucleotide. In any of the preceding embodiments, the one or more modified nucleotide or nucleotide analog residues can have a C3′ endo pucker conformation.

In any of the preceding embodiments, the one or more modified nucleotide or nucleotide analog residues can comprise one or more modified ribonucleotide residues. In some embodiments, the circular probe or circularized probe does not comprise an unmodified ribonucleotide residue. Alternatively, in some embodiments, the circular probe or circularized probe can comprise one or more unmodified ribonucleotide residues. In any of the preceding embodiments, the one or more modified nucleotide or nucleotide analog residues can be selected from the group consisting of 2′-O-methyl ribonucleic acid (2′-OMeRNA), locked nucleic acid (LNA) nucleotides, 2′-fluoro ribonucleic acid (2′-F RNA), and combinations thereof. In any of the preceding embodiments, the one or more modified nucleotide or nucleotide analog residues can comprise 2′-OMeRNA. In any of the preceding embodiments, the one or more modified nucleotide or nucleotide analog residues can comprise modified deoxyribonucleotide residues. In any of the preceding embodiments, the circular probe or circularized probe can be primarily composed of deoxyribonucleotide residues. In some embodiments, the circular probe or circularized probe can consist of deoxyribonucleotide residues. In any of the preceding embodiments, the circular probe or circularized probe may comprise no more than 20%, no more than 10%, no more than 5%, or no more than 1% of modified nucleotide or nucleotide analog residues and/or modified or unmodified ribonucleotide residues. In any of the preceding embodiments, the circular probe or circularized probe may comprise no more than two, no more than three, no more than four, or no more than five consecutive modified nucleotide or nucleotide analog residues. In any of the receding embodiments, the circular probe or circularized probe may comprise no more than two, no more than three, no more than four, or no more than five consecutive modified or unmodified ribonucleotide residues. In any of the preceding embodiments, the one or more modified nucleotide or nucleotide analog residues can comprise one or more triazole or thiol-based linkages instead of a phosphodiester group. In any of the preceding embodiments, the circular or circularizable template can comprise one or more thiol linkages between residues selected from a phosphorothioate and thiophosphate linkage. In any of the preceding embodiments, the method can comprise contacting the biological sample with a circularizable probe or probe set comprising one or more thiol linkages, wherein the one or more thiol linkages are not located at a 5′ or 3′ end of the circularizable probe or probe set. In any of the preceding embodiments, the circular probe or circularizable probe or probe set can comprise two or more triazole linkages. In any of the preceding embodiments, the circular probe or circularizable probe or probe set can comprise two or more thiol linkages. In any of the preceding embodiments, at least two of the two or more triazole linkages or the two or more thiol linkages can be separated by fewer than 100 nucleotides. In any of the preceding embodiments, the circular probe or circularizable probe or probe set can be a first circular probe or circularizable probe or probe set that hybridizes to a first target nucleic acid in the sample, and the method can further comprise: contacting the biological sample with a second circular or circularizable probe or probe set, wherein the second circular or circularizable probe or probe set hybridizes to a second target nucleic acid in the biological sample, and using a polymerase to perform rolling circle amplification (RCA) of the second circular probe or of a second circularized probe generated from the second circularizable probe or probe set, thereby generating a second RCA product. In some embodiments, the second circular probe or circularizable probe or probe set may not comprise modified nucleotide or nucleotide analog residues. In some embodiments, the second circular probe or circularizable probe or probe set can comprise fewer modified nucleotide or nucleotide analog residues than the first circular probe or circularizable probe or probe set. In some embodiments, the second circular probe or circularizable probe or probe set can comprise different modified nucleotide or nucleotide analog residues than the first circular probe or circularizable probe or probe set. In some embodiments, the second circular probe or circularizable probe or probe set does not comprise modified nucleotide or nucleotide analog residues. In any of the preceding embodiments, the polymerization rate of the polymerase on the first circular or circularized probe can be slower than the polymerization rate of the polymerase on the second circular or circularized probe. In any of the preceding embodiments, the method can comprise contacting the biological sample with a reaction mixture comprising one or more nucleotides and/or nucleotide analogs, wherein the one or more nucleotides and/or nucleotide analogs comprise: (i) a non-incorporable nucleotide or nucleotide analog that binds transiently to the polymerase but is not incorporated by the polymerase, and/or (ii) an incorporable nucleotide or nucleotide analog that is incorporated by the polymerase at a slower rate than a corresponding nucleoside triphosphate.

In some aspects, provided herein is a method for analyzing a biological sample, comprising: (a) contacting the biological sample with a reaction mixture comprising one or more nucleotides or analogs thereof, and (b) performing rolling circle amplification (RCA) of a circular nucleic acid template in the biological sample using a polymerase, thereby generating an RCA product, wherein the one or more nucleotides or analogs thereof comprise: (i) a non-incorporable nucleotide or analog thereof that bind transiently to the polymerase but is not incorporated by the polymerase, and/or (ii) an incorporable nucleotide or analog thereof that is configured to be incorporated by the polymerase at a slower rate than a corresponding nucleoside triphosphate. In some embodiments, the one or more nucleotides or analogs thereof can comprise a non-incorporable nucleotide or nucleotide analog that binds transiently to the polymerase but is not incorporated by the polymerase, and an incorporable nucleotide or nucleotide analog that is configured to be incorporated by the polymerase at a slower rate than a corresponding nucleoside triphosphate. In any of the preceding embodiments, the mean open time of the polymerase during polymerization can be greater for incorporation of the incorporable nucleotide or analog thereof than for incorporation of a corresponding nucleotide triphosphate, e.g., unmodified dNTP such as dATP, dTTP, dUTP, dCTP, or dGTP. In some embodiments, the mean open time of the polymerase during polymerization for incorporation of the incorporable nucleotide is at least 125%, 150%, 200%, 225%, or 250% of the mean open time for incorporation of a corresponding nucleoside triphosphate. In any of the preceding embodiments, the incorporable nucleotide or analog thereof can comprise an alpha-thiol nucleotide. Additionally or alternatively, in any of the preceding embodiments, the incorporable nucleotide or analog thereof can comprise a diphosphate nucleotide. Additionally or alternatively, in any of the preceding embodiments, the incorporable nucleotide can comprise an azide modified nucleotide or nucleotide analog and/or an alkyne modified nucleotide or nucleotide analog. In some embodiments wherein the one or more incorporable nucleotides include click functionalized nucleotides, the method does not comprise performing a click reaction. In any of the preceding embodiments, the reaction mixture can comprise one or more incorporable nucleotides or incorporable nucleotide analogs, wherein the incorporable nucleotides or incorporable nucleotide analogs are selected from alpha thiol nucleotides, diphosphate nucleotides, azide modified nucleotides or nucleotide analogs, alkyne modified nucleotides or nucleotide analogs, and combinations thereof. In some embodiments, the reaction mixture does not comprise unmodified deoxyribose nucleotide triphosphates. In some embodiments, the method does not comprise contacting the biological sample with unmodified deoxyribose nucleotide triphosphates. In some embodiments, no more than 50%, 40%, 30%, 20%, 10%, 5% or 1% of the nucleotides or analogs thereof in the reaction mixture are the non-incorporable nucleotide or analog thereof and/or the incorporable nucleotide or analog thereof. For example, at least 50%, 60%, 70%, 80%, 90%, 95%, or 99% of the nucleotides or analogs thereof in the reaction mixture can be unmodified deoxyribonucleotides. In any of the preceding embodiments, the non-incorporable nucleotide or analog thereof can be a monophosphate nucleotide. In any of the preceding embodiments, the non-incorporable nucleotide analog can be dissociable from the polymerase. In any of the preceding embodiments, the mean rate of polymerization by the polymerase in the rolling circle amplification n can be inversely correlated with the concentration of the one or more incorporable or non-incorporable nucleotides or nucleotide analogs in the reaction mixture. In any of the preceding embodiments, the mean rate of polymerization by the polymerase in the rolling circle amplification can be less than 2280 nt/min, less than 2000 nt/min, less than 1500 nt/min, less than 1250 nt/min, less than 1000 nt/min, less than 750 nt/min, less than 500 nt/min, or less than 250 nt/min. In any of the preceding embodiments, the rolling circle amplification can be performed at a temperature between 18° C. and 30° C. In some embodiments, the rolling circle amplification is performed at 30° C.

In some aspects, provided herein is a method for analyzing a biological sample, comprising: (a) contacting the biological sample with a reaction mixture comprising one or more nucleotide analogs, and (b) performing rolling circle amplification (RCA) of a circular nucleic acid template in the biological sample using a polymerase, thereby generating an RCA product incorporating the one or more nucleotide analogs, wherein the one or more nucleotide analogs comprise a nucleotide analog comprising a hydrophobic modification. In some embodiments, the hydrophobic modification can be a base modification. In some embodiments, the hydrophobic modification can comprise a carbon chain. In some embodiments, the hydrophobic modification can comprise a triple bond. In some embodiments, the hydrophobic modification can comprise vinyl or ethynyl group. In some embodiments, nucleotide analog comprising the hydrophobic modification can be an ethynyl-dUTP or a vinyl-dUTP. In some such embodiments, comprising the hydrophobic modification is a 5-ethynyl-dUTP or a 5-vinyl-dUTP. In some embodiments, the diameter of the RCA product generated using the one or more nucleotide analogs can be smaller than a reference RCA product produced using the same template without including the one or more nucleotide analogs in the reaction mixture. In some embodiments, the nucleotide analog comprising the hydrophobic modification can be added to the sample at a concentration of at least 1 μM, at least 1.25 μM, at least 2.5 μM, at least 5 μM, at least 10 μM, at least 40 μM, at least 80 μM, or at least 100 μM. In some embodiments, the nucleotide analog can be a modified dUTP and the ratio of the modified dUTP to an unmodified dUTP or dTTP in the reaction mixture can be between about 80:20 and about 1:99, optionally wherein the ratio of the modified dUTP to the unmodified dUTP or dTTP in the reaction mixture can be between about 80:20 and about 40:60. In some embodiments, the nucleotide analog comprising the hydrophobic modification can be added to the sample at a concentration of about 50 μM to about 100 μM, optionally wherein the nucleotide analog comprising the hydrophobic modification can be added to the sample at a concentration of about 80 μM to about 100 μM. In some embodiments, the median diameter of an RCA product generated using the one or more nucleotide analogs can be smaller than the median diameter of a reference RCA product produced using the same template without including the one or more nucleotide analogs in the reaction mixture. In some such embodiments, the median diameter of an RCA product generated using the one or more nucleotide analogs can be less than 500 nm. In some embodiments, the RCA product can be in the form of a nanoball having a diameter of between about 0.1 μm and about 3 μm, optionally wherein the diameter is between about 0.1 μm and about 0.5 μm, between about 0.5 μm and about 1 μm, between about 0.8 μm and about 1.3 μm, or between about 1 μm and about 1.5 μm. In some embodiments, the median diameter of an RCA product generated using the one or more nucleotide analogs can be no more than 90% or no more than 80% of the median diameter of a reference RCA product produced using the same template without including the one or more nucleotide analogs in the reaction mixture. In some embodiments, the average intensity, average signal-to-noise ratio, and/or density of an RCA product incorporating the one or more nucleotide analogs can be not significantly different from the average intensity, average signal-to-noise ratio, and/or density of a reference RCA product produced using the same template without including the one or more nucleotide analogs in the reaction mixture. In some embodiments, the diameter of the RCA product is smaller than the diameter of the reference RCA product. In some embodiments, the diameter of the RCA product is an average diameter and the diameter of the reference RCA product is an average diameter. In some embodiments, the diameter of the RCA product is no more than 90%, no more than 80%, or no more than 70%, of the diameter of the reference RCA product. In some embodiments, the diameter of the RCA product is less than 700 nm, less than 600 nm, or less than 500 nm. In some embodiments, the method comprises detecting an RCA product signal associated with the RCA product, and the size of the RCA product is determined based on the RCA product signal. In some embodiments, the method does not comprise crosslinking the RCA product to itself, to one or more other molecules in the biological sample, and/or to a matrix embedding the biological sample or molecules thereof, before the diameter of the RCA product is determined. In some embodiments, the method does not comprise crosslinking the RCA product. In some embodiments, the average intensity, average signal-to-noise ratio, and/or density of the RCA product signal is not significantly different from the average intensity, average signal-to-noise ratio, and/or density of a reference RCA product signal associated with the reference RCA product. In some embodiments, the one or more nucleotide analogs in the RCA product increases overall hydrophobicity of the RCA product in comparison to the reference RCA product. In some embodiments, the incorporation of the one or more nucleotide analogs into the RCA product can increase overall hydrophobicity of the RCA product. In some embodiments, the incorporation of the one or more nucleotide analogs into the RCA product can promote base stacking interactions among nucleotides in the RCA product. In any of the preceding embodiments, the RCA product can be generated using linear RCA, branched RCA, dendritic RCA, or any combination thereof. In any of the preceding embodiments, the RCA product can be generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and a variant or derivative thereof. In some embodiments, the polymerase is a Phi29 polymerase, a Vent DNA polymerase, or a Bst DNA polymerase. In some embodiments, the polymerase is a Phi29 polymerase. In any of the preceding embodiments, the RCA product can be generated in situ in the biological sample or in a matrix embedding the biological sample or molecules thereof. In any of the preceding embodiments, the RCA product can be crosslinked to one or more other molecules in the biological sample and/or a matrix embedding the biological sample or molecules thereof. In any of the preceding embodiment, the method can comprise detecting the RCA product in situ in the biological sample or in a matrix embedding the biological sample or molecules thereof. In some embodiments, detecting the RCA product can comprise contacting the biological sample with one or more detectably-labeled probes that directly or indirectly hybridize to one or more barcode sequences comprised by the RCA product. In some embodiments, a signal associated with the RCA product can be amplified in situ in the biological sample or in a matrix embedding the biological sample or molecules thereof. In some embodiments, the signal amplification can comprise rolling circle amplification (RCA) of a probe that directly or indirectly binds to the RCA product; hybridization chain reaction (HCR) directly or indirectly on the RCA product; linear oligonucleotide hybridization chain reaction (LO-HCR) directly or indirectly on the RCA product; primer exchange reaction (PER) directly or indirectly on the RCA product; assembly of branched structures directly or indirectly on the RCA product; hybridization of a plurality of detectable probes directly or indirectly on the RCA product, or any combination thereof. In any of the preceding embodiments, the method can further comprise terminating the rolling circle amplification by heat denaturation or by contacting the biological sample with a polymerase inhibitor. In some embodiments, terminating the rolling circle amplification can comprise contacting the biological sample with a polymerase inhibitor selected from the group consisting of a pyrophosphate analog, an allosteric inhibitor of the polymerase, a non-catalytic ion that binds to the polymerase, and a chain terminating nucleotide. In any of the preceding embodiments, the circular nucleic acid template can be a circular probe or a circularized probe generated from a circularizable probe or probe set, wherein the circular probe or circularizable probe or probe set hybridizes to a target nucleic acid in the biological sample. In some embodiments, the circularized probe can be generated from the circularizable probe or probe set using the target nucleic acid as a ligation template. In some embodiments, the circularizable probe set can comprise two, three, or more probes. In some embodiments, the circularized probe can be generated using enzymatic ligation and/or chemical ligation. In some embodiments, the circularized probe can be generated using template dependent ligation and/or template independent ligation. In any of the preceding embodiments, the circularized probe can be generated using a ligase having an RNA-templated DNA ligase activity and/or an RNA-templated RNA ligase activity. In any of the preceding embodiments, the circularized probe can generated using a ligase selected from the group consisting of a Chlorella virus DNA ligase (PBCV DNA ligase), a T4 RNA ligase, a T4 DNA ligase, and a single-stranded DNA (ssDNA) ligase. In some embodiments, the circularized probe is generated using a PBCV-1 DNA ligase or variant or derivative thereof and/or a T4 RNA ligase 2 (T4 Rnl2) or variant or derivative thereof. In any of the preceding embodiments, the RCA product can comprise one or more barcode sequences or complements thereof. In some embodiments, the one or more barcode sequences or complements thereof correspond to the target nucleic acid or a portion thereof. In any of the preceding embodiments, the RCA product can comprise between about 10 and about 100, between about 100 and about 1,000, between about 1,000 and about 5,000, between about 5,000 and about 10,000, or more than 10,000 copies of the circular nucleic acid template or the circular or circularized probe. In any of the preceding embodiments, the RCA product can be in the form of a nanoball having a diameter of between about 0.1 μm and about 3 μm, optionally wherein the diameter is between about 0.1 μm and about 0.5 μm, between about 0.5 μm and about 1 μm, between about 0.8 μm and about 1.3 μm, or between about 1 μm and about 1.5 μm. In any of the preceding embodiments, the RCA product can be between about 1 and about 15 kilobases, between about 15 and about 25 kilobases, between about 25 and about 35 kilobases, between about 35 and about 45 kilobases, between about 45 and about 55 kilobases, between about 55 and about 65 kilobases, between about 65 and about 75 kilobases, or more than 75 kilobases in length. In any of the preceding embodiments, the target nucleic acid can comprise DNA and/or RNA. In some embodiments, the target nucleic acid can be genomic DNA/RNA, mRNA, cDNA, or a reporter oligonucleotide of a labelling agent that directly or indirectly binds to an analyte in the biological sample. In any of the preceding embodiments, the method can further comprise crosslinking the RCA product to itself, to one or more other molecules in the biological sample, and/or to a matrix embedding the biological sample or molecules thereof. In some embodiments, the crosslinking reduces the mobility of the amplification product in the biological sample and/or in the matrix. In any of the preceding embodiments, the biological sample can be a fixed and/or permeabilized biological sample. In any of the preceding embodiments, the biological sample can be a non-homogenized tissue sample or a tissue section. In some embodiments, the biological sample can be a formalin-fixed, paraffin-embedded (FFPE) tissue sample, a frozen tissue sample, or a fresh tissue sample. In some embodiments, the biological sample can be a tissue slice between about 1 μm and about 50 μm in thickness, optionally wherein the tissue slice is between about 5 μm and about 35 μm in thickness. In some embodiments, the biological sample can be crosslinked. In some embodiments, the biological sample can be embedded in a matrix, optionally wherein the matrix is a hydrogel. In some embodiments, the biological sample can be cleared.

In some aspects, provided herein is a method for analyzing a biological sample, comprising: (a) contacting the biological sample with (i) a first circular probe or circularizable probe or probe set comprising one or more modified nucleotide or nucleotide analog residues, wherein the first circular probe or circularizable probe or probe set hybridizes to a first target nucleic acid in the biological sample, and (ii) a second circular probe or circularizable probe or probe set, wherein the second circular probe or circularizable probe or probe set hybridizes to a second target nucleic acid in the biological sample, and (b) (i) using a polymerase to perform rolling circle amplification (RCA) of the first circular probe or of a first circularized probe generated from the first circularizable probe or probe set, thereby generating a first RCA product, and (ii) using a polymerase to perform rolling circle amplification (RCA) of the second circular probe or of a second circularized probe generated from the second circularizable probe or probe set, thereby generating a second RCA product. In some embodiments, the presence of the one or more modified nucleotide or nucleotide analog residues decreases the polymerization rate of the polymerase on the first circular or circularized probe as compared to a reference circular template without the one or more modified nucleotide or nucleotide analog residues. In some embodiments, the polymerization rate of the polymerase on the first circular or circularized probe is slower than the polymerization rate of the polymerase on the second circular or circularized probe.

In some embodiments, the second circular probe or circularizable probe or probe set does not comprise modified nucleotide or nucleotide analog residues. In some embodiments, the second circular probe or circularizable probe or probe set comprises fewer modified nucleotide or nucleotide analog residues than the first circular probe or circularizable probe or probe set. In some embodiments, the first target nucleic acid is more abundant in the biological sample than the second target nucleic acid.

In some aspects, provided herein is a kit for performing rolling circle amplification, the kit comprising: (a) a circular probe or circularizable probe or probe set comprising one or more modified nucleotide or nucleotide analog residues, wherein the circular probe or circularizable probe or probe set comprises a target hybridization region complementary to a target sequence of a target nucleic acid; and (b) a polymerase; wherein the one or more modified nucleotide or nucleotide analog residues decreases the polymerization rate of the polymerase in a rolling circle amplification reaction on the circular probe or a circularized probe generated from the circularizable probe or probe set as compared to a reference circular template without the one or more modified nucleotide or nucleotide analog residues. In some embodiments, the kit can further comprise a ligase for generating the circularized probe from the circularizable probe or probe set. In some embodiments, the one or more modified nucleotide or nucleotide analog residues can comprise modified sugar nucleotide residues and/or modified backbone nucleotide residues. In some embodiments, the one or more modified nucleotide or nucleotide analog residues can be selected from the group consisting of 2′-O-methyl ribonucleic acid (2′-OMeRNA), locked nucleic acid (LNA) nucleotides, 2′-fluoro ribonucleic acid (2′-F RNA), phosphorothioate backbone nucleotides, thiophosphate backbone nucleotides, triazole-modified nucleotides, and combinations thereof. In any of the preceding embodiments, the kit can further comprise one or more non-incorporable nucleotides or nucleotide analogs configured to bind transiently to the polymerase but not be incorporated by the polymerase, and/or one or more incorporable nucleotides or nucleotide analogs configured to be incorporated by the polymerase at a slower rate than a corresponding nucleoside triphosphate, optionally wherein the one or more non incorporable nucleotides. In some aspects, provided herein is a kit for rolling circle amplification, the kit comprising: (a) a polymerase; (b) one or more non-incorporable nucleotides or nucleotide analogs configured to bind transiently to the polymerase but not be incorporated by the polymerase, and/or one or more incorporable nucleotides or nucleotide analogs configured to be incorporated by the polymerase at a slower rate than a corresponding nucleoside triphosphate; and (c) one or more circular probes or circularizable probes or probe sets for generating a circularized probe as a template for rolling circle amplification, or one or more reagents for generating a circularized template for rolling circle amplification. In any of the preceding embodiments, the one or more non-incorporable nucleotides or nucleotide analogs and/or one or more incorporable nucleotides or nucleotide analogs can comprise an alpha-thiol nucleotide, a diphosphate nucleotide, and/or a monophosphate nucleotide. In any of the preceding embodiments, the kit can further comprise unmodified deoxyribose nucleotide triphosphates. In any of the preceding embodiments of the kit, the combination of the one or more the one or more non-incorporable nucleotides or nucleotide analogs, the one or more incorporable nucleotides or nucleotide analogs, and/or the unmodified deoxyribose nucleotide triphosphates can be comprised by a reaction mixture for rolling circle amplification. In some aspects, provided herein is a kit for performing rolling circle amplification, the kit comprising: (a) a polymerase; (b) one or more nucleotide analogs comprising one or more hydrophobic modifications configured to be incorporated by the polymerase; and (c) one or more circular probes or circularizable probes or probe sets for generating the circularized probe as a template for rolling circle amplification, or one or more reagents for generating a circularized template for rolling circle amplification. In some embodiments, the one or more nucleotide analogs can comprise a modified dUTP selected from ethynyl-dUTP and/or vinyl dUTP. In some embodiments, the one or more nucleotide analogs can be provided as a reaction mixture comprising the one or more nucleotide analogs and unmodified deoxyribose nucleotide triphosphates. In some embodiments, the ratio of the modified dUTP to an unmodified dTTP in the reaction mixture can be at least about 80:20.

### BRIEF DESCRIPTION OF THE DRAWINGS

The drawings illustrate certain embodiments of the features and advantages of this disclosure. These embodiments are not intended to limit the scope of the appended claims in any manner.

FIGS. 1A-1B show schematics illustrating exemplary circular or circularized probes with (FIG. 1A) or without (FIG. 1B) one or more modified nucleotide or nucleotide analog residues, and rolling circle amplification (RCA) of the probes. The one or more modified nucleotide or nucleotide analog residues can reduce the rate of polymerization during RCA, thereby resulting in a smaller RCA product compared to probes with unmodified nucleotide residue(s) (e.g., residues with A, T, C, or G bases, and unmodified/natural sugar phosphate backbones) at the corresponding positions in the probes, under otherwise equal RCA conditions (e.g., reaction temperature, time, etc.). FIG. 1C shows the structure of unmodified DNA residue and an exemplary modified RNA residue with a modified sugar moiety (2′-OMeRNA). FIG. 1D shows the structure of an exemplary modified nucleotide or nucleotide analog residue or nucleotide analog comprising a modified backbone that forms a triazole linkage with another residue.

FIG. 2A shows a schematic illustrating an exemplary method for analyzing a biological sample using RCA with a reaction mixture comprising (i) a non-incorporable nucleotide (e.g., dNMP or a modified nucleotide) or nucleotide analog that is not incorporated (e.g., the non-incorporable nucleotide or nucleotide analog is configured to only bind transiently to a polymerase and not be incorporated into an RCA product), and/or (ii) an incorporable nucleotide or nucleotide analog that is configured to be incorporated by the polymerase at a slower rate than a corresponding nucleoside triphosphate (e.g., a dNTP with an A, T/U, C, or G base and an unmodified/natural sugar phosphate backbone). The reaction mixture can include unmodified dNTPs, as shown. FIG. 2B shows exemplary structures of (i) an exemplary non-incorporable nucleotide (e.g., deoxyribonucleoside monophosphate, dNMP) that can bind transiently to a polymerase and compete with other, incorporable nucleotides for binding to the polymerase, and (ii) exemplary incorporable nucleotides or nucleotide analogs (e.g., deoxyribonucleoside diphosphate (dNDP) and an alpha thiol nucleotide), that are incorporated by the polymerase at a slower rate than a corresponding dNTP.

FIG. 3 shows a plot indicating the density of detected in situ RCA products (detected object count per unit nuclei area) for the indicated experimental conditions. Concentrations of modified dNTPs (5-Azido-PEG₄-dCTP and 5-Ethynyl-dUTP) are shown as a percentage of total dNTPs. Dots represent densities from experimental replicates.

FIG. 4 shows a plot indicating the mean size of detected in situ RCA product signals for the indicated experimental conditions. Concentrations of modified dNTPs (5-Azido-PEG₄-dCTP and 5-Ethynyl-dUTP) are shown as a percentage of total dNTPs. Dots represent mean sizes from experimental replicates.

FIG. 5 shows a plot indicating mean signal intensity above local background for in situ RCA product signals for the indicated experimental conditions. Concentrations of modified dNTPs (5-Azido-PEG₄-dCTP and 5-Ethynyl-dUTP) are shown as a percentage of total dNTPs. Dots represent mean intensities from experimental replicates.

FIG. 6A shows the molecular structure of modified dNTPs 5-Azido-PEG₄-dCTP and 5-Ethynyl-dUTP used in the in situ RCA and quantitative RCA experiments described herein. FIG. 6B shows results of quantitative rolling circle amplification (qRCA) with inclusion of 0%, 50%, or 100% of the indicated dNTPs (5-Azido-PEG₄-dCTP and 5-Ethynyl-dUTP, individually or in combination), and negative controls. The y-axis corresponds to relative fluorescence units (RFU), x-axis corresponds to imaging cycles over time.

FIG. 7 shows the molecular structure of modified dNTPs 5-Ethynyl-dUTP (5-EdUTP) and 5-Vinyl-dUTP used in the in situ RCA experiments described herein. Dashed circles indicate added hydrophobic modifications (e.g., hydrophobic groups).

FIGS. 8A-8D show results from RCA experiments including indicated concentrations of the modified dNTPs: 5-Ethynyl-dUTP (5-EdUTP) or 5-Vinyl-dUTP. Control (Ctrl) conditions did not include the modified dNTP. FIG. 8A shows bar plots indicating density of detected RCA products (RCA products detected per area of nucleus imaged) for the indicated experimental conditions. No data for 40 μM 5-EdUTP condition. FIG. 8B shows bar plots indicating signal intensity of detected RCA products (mean signal intensity above local background) for the indicated experimental conditions. FIG. 8C shows bar plots indicating signal-to-noise ratio for detected RCA products (mean local signal-to-noise ratio mean) for the indicated experimental conditions. FIG. 8D shows bar plots indicating signal intensity of detected RCA products (mean signal-to-background ratio mean) for the indicated experimental conditions.

FIGS. 9A-9B show results from RCA experiments including indicated concentrations of the modified dNTPs: 5-Ethynyl-dUTP (5-EdUTP, FIG. 9A) or 5-Vinyl-dUTP (FIG. 9B). Control (Ctrl) conditions did not include the modified dNTP. Violin plots indicate size of detected RCA products for indicated experimental conditions. Dots represent size of individual detected RCA products, shapes represent size distributions.

FIGS. 10A-10B show results from RCA experiments including indicated concentrations of the modified dNTPs: 5-Ethynyl-dUTP (5-EdUTP, FIG. 10A) or 5-Vinyl-dUTP (FIG. 10B). Control (Ctrl) conditions did not include the modified dNTP. Plots represent size distribution of detected RCA products for indicated conditions, in terms of probability (top panels) and cumulative probability (middle panels). Tables (bottom panels) show summary statistics from replicates for each condition. Results indicate a decrease in RCA product size with modified dNTPs comprising a hydrophobic modification.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

In assays involving in situ rolling circle amplification (RCA), the heterogeneous size and intensity distribution of rolling circle amplification products (RCPs) can lead to loss of sensitivity, because weak signals associated with certain RCPs may not pass the detection threshold of RCP signal (e.g., “spot”) detection for image analysis, while strong signals associated with certain RCPs in close proximity may lead to optical crowding as well as the inability to detect nearby weaker signal spots. Both weak undetected RCPs and large overcrowding RCPs can lead to loss of sensitivity. In some aspects, the ability to tune the rate of polymerization would permit much more control over the RCA reaction and the resulting diameter of the RCP signal spots. For example, increased control over the rate of RCA could make it easier to terminate an RCA reaction at the time point when the RCA product is an optimal size for detection (e.g., as small as the detection limits will allow).

Controlling the rate of polymerization for an RCA reaction (e.g., an RCA reaction performed in situ in a biological sample such as a tissue sample) can be challenging for a number of reasons. Polymerization rate can be dependent on temperature. For example, the Phi29 polymerase has a rate of about 2280 nt/min at 30° C., about 1490 nt/min at 15° C., and about 290 nt/mn at 4° C. (Soengas, et al. 1995 J. Mol. Biol. 253(4):517-529, the content of which is herein incorporated by reference in its entirety). Thus, dropping the reaction temperature for RCA can in theory have a dramatic effect on the rate of polymerization. However, for some applications, it may be desirable to control the rate of polymerization for RCA without changing the temperature, e.g., to reduce the complexity of instrumentation required to control temperature, and/or to tune polymerization rates for RCA performed on multiple samples at the same temperature. Additionally, it may not be feasible to perform RCA at some lower temperatures, for example because of precipitation of buffer or sample preservative components (e.g., tissue preservative or clearing agents).

In some aspects, the present disclosure provides methods and compositions for altering the rate of rolling circle amplification using RCA templates (e.g., circular or circularized probes) comprising one or more modified nucleotide or nucleotide analog residues, wherein the one or more modified nucleotides slow the polymerization rate of the polymerase on the template. In some embodiments, provided herein is a method for analyzing a biological sample, comprising: (a) contacting the biological sample with a circular probe or circularizable probe or probe set comprising one or more modified nucleotide or nucleotide analog residues, wherein the circular probe or circularizable probe or probe set hybridizes to a target nucleic acid in the biological sample, and (b) using a polymerase to perform rolling circle amplification (RCA) of the circular probe or of a circularized probe generated from the circularizable probe or probe set, thereby generating an RCA product, wherein the presence of the one or more modified nucleotide or nucleotide analog residues in the circular probe or circularized probe decreases the polymerization rate of the polymerase using the circular probe or circularized probe as template as compared to a reference circular template without the one or more modified nucleotide or nucleotide analog residues. In some embodiments, the reference circular template is a template that does not comprise the one or more modified nucleotide or nucleotide analog residues, but is otherwise identical to the circular or circularized probe. For example, the reference circular template may have the same sequence as the circular or circularized probe, but includes unmodified nucleotide or nucleotide analog residues in place of the one or more modified nucleotide or nucleotide analog residues of the circular or circularized probe.

The one or more modified nucleotide or nucleotide analog residues can comprise, for example, sugar modified nucleotides and/or nucleotide residues comprising triazole or thiol-based linkages. In some embodiments, the one or more modified nucleotides can comprise C2′ modified nucleotides. In some embodiments, the one or more modified nucleotide or nucleotide analog residues can have a C3′ endo pucker conformation. The one or more modified nucleotide or nucleotide analog residues can comprise one or more modified ribonucleotide or deoxyribonucleotide residues. Examples of sugar modified nucleotide or nucleotide analog residues include but are not limited to 2′-OMeRNA, locked nucleic acid (LNA) nucleotides, 2′-F RNA, and combinations thereof. In some embodiments, the circular probe or circularizable probe or probe set comprises one or more thiol linkages between residues, such as a phosphorothioate or thiophosphate linkage. In some embodiments, the circular probe or circularizable probe or probe set comprises two or more triazole linkages and/or thiol linkages. In some embodiments, the two or more triazole linkages and/or thiol linkages are separated by fewer than 100 nucleotides.

In some embodiments, the one or more modified nucleotide or nucleotide analog residues include 2′-OMeRNA (e.g., 1, 2, 3, 4, or more 2′-OMeRNA residues). A single 2′-OMeRNA residue included in an exemplary circular template has been shown to reduce the rate of rolling circle amplification by about 90% for Phi29 polymerase, about 40% for Vent DNA polymerase, and about 30% for Bst DNA polymerase. For this reason, it has been recommended that use of 2′-OMeRNA should be avoided in RCA applications. See, for example, Tang et al. (2016) Bioscience, Biotechnology, and Biochemistry, 80:8, 1555-1561, the content of which is herein incorporated by reference in its entirety. Some aspects of the present disclosure are thus based in part on the realization that the strong suppression of the rate of polymerization using a template including 2′-OMeRNA may be advantageous in certain applications, such as in situ detection of analytes by RCA. In some embodiments, the circular probe or circularizable probe or probe set comprises between or between about any of 1 and 10, 1 and 5, 1 and 4, 1 and 3, 1 and 2, 2 and 10, 2 and 5, 2 and 4, or 5 and 10 2′-OMeRNA residues. In some embodiments, the circular probe or circularizable probe or probe set comprises 1, 2, 3, 4, 5, 6, 7, 8, 9, 10, or more 2′-OMeRNA residues.

In some aspects, the present disclosure provides methods that facilitate differential tuning of polymerization rates in RCA reactions among different circular probes or circularizable probes or probe sets, because the polymerization rate in an RCA reaction can be linked to modified nucleotide or nucleotide analog residues in the RCA template itself (such as any of the modified nucleotide or nucleotide analog residues described herein, e.g. comprising modified sugars, thiol linkages, and/or triazole linkages). In some embodiments, circular probes or circularizable probes or probe sets comprising different modified nucleotide or nucleotide analog residues and/or different numbers of modified nucleotide or nucleotide analog residues can be designed to hybridize to different target nucleic acids. For example, a circular or circularizable probe or probe set that hybridizes to an first target nucleic acid in the sample can be designed to provide a circular or circularized probe that is amplified at a slower rate than a circular or circularized probe that hybridizes to a second target nucleic acid. In such an example, the first target nucleic acid may be more abundant than the second target nucleic acid. In some embodiments, a method provided herein comprises contacting the biological sample with a first circular probe or circularizable probe or probe set that hybridizes to a first target nucleic acid in the sample, and contacting the biological sample with a second circular or circularizable probe or probe set, wherein the second circular or circularizable probe or probe set hybridizes to a second target nucleic acid in the biological sample, and using a polymerase to perform rolling circle amplification (RCA) of (i) the first circular probe or of a first circularized probe generated from the first circularizable probe or probe set, and (ii) the second circular probe or of a second circularized probe generated from the second circularizable probe or probe set, thereby generating a first RCA product and a second RCA product. In some embodiments, the polymerization rate of the polymerase on the first circular or circularized probe is slower than the polymerization rate of the polymerase on the second circular or circularized probe. In some embodiments, the first RCA product is smaller than the second RCA product. In some embodiments, the first circular probe or circularizable probe or probe set comprises one or more modified nucleotides, and the second circular probe or circularizable probe or probe set does not comprise modified nucleotide or nucleotide analog residues. In other embodiments, both the first and second circular probe or circularizable probe or probe set can comprise one or more modified nucleotides. In some embodiments, the second circular probe or circularized probe comprises fewer modified nucleotide or nucleotide analog residues than the first circular probe or circularized probe. In some embodiments, the second circular probe or circularized probe comprises different modified nucleotide or nucleotide analog residues than the first circular probe or circularized probe.

In some aspects, the present disclosure provides methods of tuning the rate of polymerization by a polymerase in an RCA reaction by including one or more molecules in a reaction mixture that decrease the rate of polymerization. In some embodiments, provided herein is a method for analyzing a biological sample, comprising: (a) contacting the biological sample with a reaction mixture comprising one or more nucleotides or analogs thereof, and (b) performing rolling circle amplification (RCA) of a circular nucleic acid template in the biological sample using a polymerase, thereby generating an RCA product, wherein the one or more nucleotides or analogs thereof comprise: (i) a non-incorporable nucleotide or analog thereof that binds transiently to the polymerase but is not incorporated by the polymerase, and/or (ii) an incorporable nucleotide or analog thereof that is configured to be incorporated by the polymerase at a slower rate than a corresponding nucleoside triphosphate. The circular template can be any suitable circular template for rolling circle amplification, such as a circular probe, a circularized probe generated from a circularizable probe or probe set, or a circularized cDNA molecule. Circularized cDNA molecules are used as circular nucleic acid templates for RCA in methods such as FISSEQ, for example as described in Lee et al. Science. 2014; 343(6177):1360-1363, the content of which is herein incorporated by reference in its entirety.

In some embodiments, the rate of RCA can be modified using any combination of the methods and compositions described herein. The provided methods for analyzing a biological sample can include (a) use of a circular or circularized probe as a template for RCA comprising one or more modified nucleotide or nucleotide analog residues, wherein the inclusion of modified nucleotides in the template decreases the polymerization rate of the polymerase using the circular or circularized probe as template as compared to a reference circular template; (b) contacting the biological sample with a reaction mixture for rolling circle amplification comprising one or more non-incorporable nucleotides or analogs thereof (e.g., analogs that are themselves non-incorporable), wherein the non-incorporable nucleotide or analog thereof binds transiently to the polymerase but is not incorporated by the polymerase; and/or (c) contacting the biological sample with a reaction mixture for rolling circle amplification comprising an incorporable nucleotide or analog thereof (e.g., an analog that is itself incorporable), wherein the incorporable nucleotide or analog thereof is configured to be incorporated by the polymerase at a slower rate than a corresponding nucleoside triphosphate, either individually or in any combination of (a), (b), and (c).

In some embodiments, the present disclosure provides methods of tuning the size of RCA products by including one or more nucleotide analogs comprising a hydrophobic modification in a reaction mixture for RCA. In some embodiments, the RCA product incorporates the one or more nucleotide analogs comprising hydrophobic modifications, and the hydrophobic modifications within the RCA product lead to a reduced overall size of the RCA product, for example as compared to a reference RCA product generated in the absence of the nucleotide analogs. In some embodiments, incorporation of the one or more nucleotide analogs into the RCA product can promote base stacking interactions among nucleotides in the RCA product, thereby reducing the overall size of the RCA product. In some embodiments, provided herein is a method for analyzing a biological sample comprising: (a) contacting the biological sample with a reaction mixture comprising one or more nucleotide analogs, and (b) performing rolling circle amplification (RCA) of a circular nucleic acid template in the biological sample using a polymerase, thereby generating an RCA product incorporating the one or more nucleotide analogs, wherein the one or more nucleotide analogs comprise a nucleotide analog comprising a hydrophobic modification. In some embodiments, the hydrophobic modification is a base modification. In some embodiments, the hydrophobic modification comprises a carbon chain and/or hydrocarbon ring, a triple bond, and/or a vinyl or ethynyl group. In some embodiments, the nucleotide analog comprising the hydrophobic modification is an ethynyl-dUTP or a vinyl-dUTP, such as 5-ethynyl-dUTP or a 5-vinyl-dUTP, respectively.

In the following sections, additional description of various aspects of the methods, compositions, and kits disclosed herein is provided. Section II describes exemplary biological samples as well as analytes (e.g., endogenous analytes, labelling agents, or products of endogenous nucleic analytes) that can be detected using a method of analyzing a biological sample described herein (e.g., by detecting a target nucleic acid that is or is associated with the analyte). Section III describes exemplary circular probes or circularizable probes or probe sets that can be circularized for use as RCA templates according to the methods provided herein. Section IV describes RCA reactions and reaction mixtures. Section V describes nucleotides, nucleotide analogs, and nucleotide modifications in connection with the methods and compositions described herein. Section VI describes methods for detecting RCA products in the sample. Section VII provides kits according to the present disclosure. Section VIII describes exemplary applications of the present methods, compositions, and kits. As stated above, the section headings used herein are for organizational purposes only and are not to be construed as limiting the subject matter described.

### II. Samples, Analytes, and Target Sequences

A. Samples

A sample disclosed herein can be or be derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can include nucleic acids (such as DNA or RNA), proteins/polypeptides, carbohydrates, and/or a lipids. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Cell-free biological samples can include extracellular polynucleotides. Extracellular polynucleotides can be isolated from a bodily sample, e.g., blood, plasma, serum, urine, saliva, mucosal excretions, sputum, stool, and tears.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

### (ii) Freezing

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Fixation and Postfixation

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circular or circularizable probe, such as a padlock probe. In some embodiments, one or more post-fixing step is performed after a hybridization complex comprising a probe and a target is formed in a sample. In some embodiments, one or more post-fixing step is performed prior to a ligation reaction disclosed herein, such as the ligation to circularize a circularizable probe, such as a padlock probe.

In some embodiments, one or more post-fixing step is performed after contacting a sample with a binding or labeling agent (e.g., an antibody or antigen binding fragment thereof) for a non-nucleic acid analyte such as a protein analyte. The labeling agent can comprise a nucleic acid molecule (e.g., reporter oligonucleotide) comprising a sequence corresponding to the labeling agent and therefore corresponds to (e.g., uniquely identifies) the analyte. In some embodiments, the labeling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

### (iv) Embedding

As an alternative to paraffin embedding described above, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other suitable hydrogel-formation method.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 m to about 2 mm.

Additional methods and aspects of hydrogel embedding of biological samples are described for example in Chen et al., Science 347(6221):543-548, 2015, the entire contents of which are incorporated herein by reference.

### (v) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Any suitable methods of destaining or discoloring a biological sample may be utilized and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### (vi) Isometric Expansion

In some embodiments, a biological sample embedded in a matrix (e.g., a hydrogel) can be isometrically expanded. Isometric expansion methods that can be used include hydration, a preparative step in expansion microscopy, as described in, e.g., Chen et al., Science 347(6221):543-548, 2015 and U.S. Pat. No. 10,059,990, each of which are herein incorporated by reference in their entireties.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, Mass.), Label-IT Amine (available from MirusBio, Madison, Wis.) and Label X (described for example in Chen et al., Nat. Methods 13:679-684, 2016 and U.S. Pat. No. 10,059,990, the entire contents of each which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(vii) Crosslinking and De-Crosslinking

In some embodiments, the biological sample is reversibly cross-linked prior to or during an in situ assay. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible or irreversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labeling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

(viii) Tissue Permeabilization and Treatment

In some embodiments, a biological sample can be permeabilized to facilitate transfer of species (such as probes) into the sample. If a sample is not permeabilized sufficiently, the transfer of species (such as probes) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

### (ix) Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, e.g., to generate cDNA, thereby selectively enriching these RNAs.

In some aspects, when two or more analytes are analyzed, a first and second probe that is specific for (e.g., specifically hybridizes to) each RNA or cDNA analyte can be used. For example, in some embodiments of the methods provided herein, templated ligation is used to detect gene expression in a biological sample. An analyte of interest (such as a protein), bound by a labeling agent or binding agent (e.g., an antibody or epitope binding fragment thereof), wherein the binding agent is conjugated or otherwise associated with a reporter oligonucleotide comprising a reporter sequence that identifies the binding agent, can be targeted for analysis. Probes may be hybridized to the reporter oligonucleotide and ligated in a templated ligation reaction to generate a product for analysis. In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include extension or amplification of templated ligation products (e.g., by rolling circle amplification of a circular product generated in a templated ligation reaction).

biological sample may comprise one or a plurality of analytes of interest. Methods for performing multiplexed assays to analyze two or more different analytes in a single biological sample are provided.

B. Analytes

The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a padlock or other circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

### (i) Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labelling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

In certain embodiments, an analyte can be extracted from a live cell. Processing conditions can be adjusted to ensure that a biological sample remains live during analysis, and analytes are extracted from (or released from) live cells of the sample. Live cell-derived analytes can be obtained only once from the sample, or can be obtained at intervals from a sample that continues to remain in viable condition.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

In any embodiment described herein, the analyte comprises a target sequence. In some embodiments, the target sequence may be endogenous to the sample, generated in the sample, added to the sample, or associated with an analyte in the sample. In some embodiments, the target sequence is a single-stranded target sequence (e.g., a sequence in a rolling circle amplification product). In some embodiments, the analytes comprise one or more single-stranded target sequences. In one aspect, a first single-stranded target sequence is not identical to a second single-stranded target sequence. In another aspect, a first single-stranded target sequence is identical to one or more second single-stranded target sequence. In some embodiments, the one or more second single-stranded target sequence is comprised in the same analyte (e.g., nucleic acid) as the first single-stranded target sequence. Alternatively, the one or more second single-stranded target sequence is comprised in a different analyte (e.g., nucleic acid) from the first single-stranded target sequence.

### (ii) Labelling Agents

In some embodiments, provided herein are methods and compositions for analyzing endogenous analytes (e.g., RNA, ssDNA, and cell surface or intracellular proteins and/or metabolites) in a sample using one or more labelling agents. In some embodiments, an analyte labelling agent may include an agent that interacts with an analyte (e.g., an endogenous analyte in a sample). In some embodiments, the labelling agents can comprise a reporter oligonucleotide that is indicative of the analyte or portion thereof interacting with the labelling agent. For example, the reporter oligonucleotide may comprise a barcode sequence that permits identification of the labelling agent. In some cases, the sample contacted by the labelling agent can be further contacted with a probe (e.g., a single-stranded probe sequence), that hybridizes to a reporter oligonucleotide of the labelling agent, in order to identify the analyte associated with the labelling agent. In some embodiments, the analyte labelling agent comprises an analyte binding moiety and a labelling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labelling agents.

In the methods and systems described herein, one or more labelling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have coupled thereto a first reporter oligonucleotide, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or antigen binding fragments thereof. The antibodies or antigen binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labelling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labelling agents are the same. In some embodiments in which the plurality of analytes includes a single species of analyte, the analyte binding moieties of the plurality of analyte labelling agents are the different (e.g., members of the plurality of analyte labelling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide.

In some aspects, these reporter oligonucleotides may comprise nucleic acid barcode sequences that permit identification of the labelling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents may be achieved through any of a variety of direct or indirect, covalent or non-covalent associations or attachments. For example, oligonucleotides may be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labelling agents. Commercially available kits, such as those from Thunderlink and Abcam, and other suitable techniques may be used to couple reporter oligonucleotides to labelling agents as appropriate. In another example, a labelling agent is indirectly (e.g., via hybridization) coupled to a reporter oligonucleotide comprising a barcode sequence that identifies the label agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing.

In some cases, the labelling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labelling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

(iii) Products of Endogenous Analyte and/or Labelling Agent

In some embodiments, provided herein are methods and compositions for analyzing one or more products of an endogenous analyte and/or a labelling agent in a biological sample. In some embodiments, an endogenous analyte (e.g., a viral or cellular DNA or RNA) or a product thereof (e.g., a hybridization product, a ligation product, an extension product (e.g., by a DNA or RNA polymerase), a replication product, a transcription/reverse transcription product, and/or an amplification product such as a rolling circle amplification (RCA) product) is analyzed. In some embodiments, a labelling agent that directly or indirectly binds to an analyte in the biological sample is analyzed. In some embodiments, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labelling agent that directly or indirectly binds to an analyte in the biological sample is analyzed.

C. Target Sequences

A target sequence for a probe disclosed herein (e.g., a circular probe or circularizable probe or probe set) may be comprised in any analyte disclose herein, including an endogenous analyte (e.g., a viral or cellular nucleic acid), a labelling agent, or a product of an endogenous analyte and/or a labelling agent. In some embodiments, a target sequence for a probe disclosed herein (e.g., a circular probe or circularizable probe or probe set, or circularized probe) is comprised by a target nucleic acid.

In some aspects, one or more of the target sequences includes one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”). In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In some embodiments, barcodes or complements thereof (e.g., barcode sequences or complements thereof comprised by the RCA products herein) can be analyzed (e.g., detected or sequenced) using any suitable methods or techniques, including those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), in situ sequencing, hybridization-based in situ sequencing (HybISS), targeted in situ sequencing, fluorescent in situ sequencing (FISSEQ), sequencing by synthesis (SBS), sequencing by ligation (SBL), sequencing by hybridization (SBH), or spatially-resolved transcript amplicon readout mapping (STARmap). In any of the preceding embodiments, the methods provided herein can include analyzing the barcodes by sequential hybridization and detection with a plurality of labelled probes (e.g., detection oligos).

In some embodiments, in a barcode sequencing method, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4^{N} complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some embodiments, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., US 2019/0055594 and US 2021/0164039, which are hereby incorporated by reference in their entirety.

### III. Circular or Circularizable Probes or Probe Sets

In some aspects, the methods provided herein comprise performing rolling circle amplification (RCA) of a circular probe or a circularized probe generated from a circularizable probe or probe set. In some aspects, the circular probe or circularized probe comprises one or more modified nucleotide or nucleotide analog residues that decrease the rate of polymerization by a polymerase using the circular probe or circularized probe as a template for RCA as compared to a reference circular template without the one or more modified nucleotide or nucleotide analog residues. In other embodiments, the circular probe or circularized probe does not comprise modified nucleotide or nucleotide analog residues.

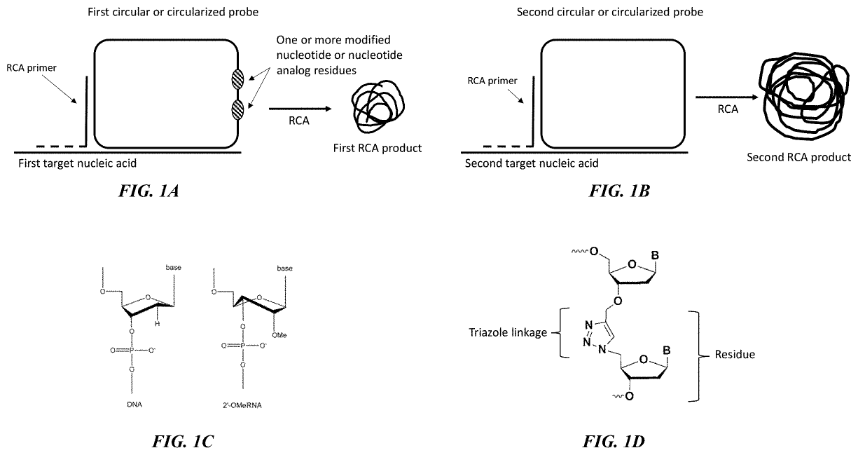

In some embodiments, a method provided herein comprises contacting the biological sample with a circular probe or circularizable probe or probe set that hybridizes to a target nucleic acid in the sample. In some embodiments, the circular probe or circularizable probe or probe set comprises one or more modified nucleotide or nucleotide analog residues. In some embodiments, the method comprises generating a circularized probe from the circularizable probe or probe set. In some embodiments, the circularized probe comprises the one or more modified nucleotide or nucleotide analog residues. In some embodiments, the circular or circularized probe is the template for the rolling circle amplification. FIG. 1A shows an exemplary first circular probe or circularized probe comprising one or more modified nucleotide or nucleotide analog residues. In some aspects, the presence of the one or more modified nucleotide or nucleotide analog residues in the circular or circularized probe decreases the polymerization rate of the polymerase using the circular or circularized probe as template as compared to a reference circular template without the one or more modified nucleotide or nucleotide analog residues. In some embodiments, a reference circular template is otherwise identical to the circular or circularized probe comprising the one or more modified nucleotide or nucleotide analog residues. For example, the reference circular template may have the same nucleotide sequence as the circular or circularized probe, but includes unmodified nucleotide residues in place of the one or more modified nucleotide or nucleotide analog residues of the circular or circularized probe.

Circularizable probes or probe sets that can be used to generate a circularized probe such as the exemplary probe shown in FIG. 1A can be any linear probe or set of linear probes that can be circularized by ligation, such as any of the circularizable probes or probe sets described below. In some embodiments, the method further comprises contacting the sample with a primer that hybridizes to the circular probe, circularizable probe or probe set, or circularized probe for performing RCA (e.g., as shown in FIG. 1A). Optionally, the primer can also comprise a region that hybridizes to the target nucleic acid, for example as indicated by the dashed line in FIG. 1A. In other embodiments, the target nucleic acid itself can prime the rolling circle amplification of the circular probe or circularized probe (e.g., as in RollFISH. See, e.g., Wu et al. Commun Biol 1, 209 (2018), which is hereby incorporated by reference in its entirety).

In some embodiments, a method provided herein comprises performing RCA of a circular or circularized probe that does not comprise modified nucleotide or nucleotide analog residues. FIG. 1B shows an exemplary second circular or circularized probe that does not comprise any modified nucleotide or nucleotide analog residues. In certain aspects, the circular or circularized probe shown in FIG. 1B can be a reference circular template, wherein the rate of polymerization by the polymerase on a corresponding circular or circularized template comprising one or more modified nucleotide or nucleotide analog residues is slower than the rate on the reference template.

In some embodiments, provided herein is a method comprising performing RCA of a first circular or circularized probe hybridized to a first target nucleic acid and comprising one or more modified nucleotide or nucleotide analog residues (e.g., as shown in FIG. 1A) and a second circular or circularized probe that is hybridized to a second target nucleic acid and does not comprise modified nucleotide or nucleotide analog residues (e.g., as shown in FIG. 1B). In some embodiments, the rate of polymerization of the polymerase on the first circular or circularized probe is slower (e.g. at least any of 10%, 20%, 30%, 40%, 50%, 60%, 70%, 80%, or 90% slower) than the rate of polymerization of the polymerase on the second circular or circularized probe. In some embodiments, the rate of polymerization of the polymerase on the first circular or circularized probe is slower than the rate of polymerization of the polymerase on the second circular or circularized probe by no more than any of 95%, 50%, 80%, 70%, 60%, 50%, 40%, 30%, 20%, or 10%. In some embodiments, the rate of polymerization of the polymerase on the first circular or circularized probe is slower than the rate of polymerization of the polymerase on the second circular or circularized probe by 5%-15%, 10%-15%, 10%-20%, 15%-20%, 10%-30%, 15%-30%, 10%-50%, 10%-70%, 10%-90%, 30%-50%, 30%-90%, 50%-70%, 70%-90%, or 50%-90%. In some embodiments, the rate of polymerization of the polymerase is the number of nucleotides incorporated per minute (nt/min), for example into the RCA product. In some embodiments, the rate of polymerization is a mean rate of polymerization. For example, a polymerase using as template a circular probe comprising both modified and unmodified nucleotide residues may incorporate nucleotides at a slower rate when using the modified residues as template than when using the unmodified residues as template. In such an example, the mean rate of polymerization using the template with modified and unmodified residues as a whole will be reduced, for example as compared to the mean rate of polymerization using a reference template lacking the modified residues. In some embodiments, a slower or reduced rate (such as a reduced mean rate) of polymerization results in a smaller RCA product.

In some aspects, the method results in a smaller (e.g., shorter) first RCA product produced from the first circular or circularized probe as compared to the second RCA product produced from the second circular or circularized probe. In any of the preceding embodiments, the first and/or second RCA product can comprise between about 10 and about 100, between about 100 and about 1,000, between about 1,000 and about 5,000, between about 5,000 and about 10,000, or more than 10,000 copies of the circular or circularized probe. In some embodiments, the first RCA product comprises fewer copies (e.g. at least any of 10%, 20%, 30%, 40%, 50%, 60%, 70%, 80%, or 90% fewer copies) of the circular or circularized probe compared to the second RCA product. In some embodiments, the first RCA product comprises no more than 95%, 50%, 80%, 70%, 60%, 50%, 40%, 30%, 20%, or 10% fewer copies of the circular or circularized probe compared to the second RCA product. In some embodiments, the first RCA product comprises 5%-15%, 10%-15%, 10%-20%, 15%-20%, 10%-30%, 15%-30%, 10%-50%, 10%-70%, 10%-90%, 30%-50%, 30%-90%, 50%-70%, 70%-90%, or 50%-90% fewer copies of the circular or circularized probe compared to the second RCA product.

In any of the preceding embodiments, the first and/or second RCA product can be in the form of a nanoball having a diameter of between about 0.1 μm and about 3 μm, optionally wherein the diameter is between about 0.1 μm and about 0.5 μm, between about 0.5 μm and about 1 μm, between about 0.8 μm and about 1.3 μm, or between about 1 μm and about 1.5 μm. In some embodiments, the first RCA product has a diameter less than (e.g. at least any of 10%, 20%, 30%, 40%, 50%, 60%, 70%, 80%, or 90% less than) the diameter of the second RCA product. In some embodiments, the first RCA product has a diameter of no more than 95%, 50%, 80%, 70%, 60%, 50%, 40%, 30%, 20%, or 10% less than the diameter of the second RCA product. In some embodiments, the first RCA product has a diameter of 5%-15%, 10%-1⁵%, 10%-20%, 15%-20%, 10%-30%, 15%-30%, 10%-50%, 10%-70%, 10%-90%, 30%-50%, 30%-90%, 50%-70%, 70%-90%, or 50%-90% less than the diameter of the second RCA product. In some aspects, the determined diameter of an RCA product may vary depending on how the three-dimensional RCA product is measured. In some aspects, the determined diameter of an RCA product is represented by the maximum measured diameter, the minimum measured diameter, an average measured diameter of the RCA product, or any other suitable method of measuring the diameter of the RCA product. In some aspects, the size (e.g., diameter) of the RCA product is measured based on a signal (e.g. a fluorescent signal) generated from the RCA product.

In any of the preceding embodiments, the RCA product can be between about 1 and about 15 kilobases, between about 15 and about 25 kilobases, between about 25 and about 35 kilobases, between about 35 and about 45 kilobases, between about 45 and about 55 kilobases, between about 55 and about 65 kilobases, between about 65 and about 75 kilobases, or more than 75 kilobases in length. In some embodiments, length of the first RCA product is shorter (e.g. at least any of 10%, 20%, 30%, 40%, 50%, 60%, 70%, 80%, or 90% shorter) than the length of the second RCA product. In some embodiments, the length of the first RCA product is shorter than the length of the second RCA product by no more than any of 95%, 50%, 80%, 70%, 60%, 50%, 40%, 30%, 20%, or 10%. In some embodiments, the length of the first RCA product is shorter than the length of the second RCA product by 5%-15%, 10%-15%, 10%-20%, 15%-20%, 10%-30%, 15%-30%, 10%-50%, 10%-70%, 10%-90%, 30%-50%, 30%-90%, 50%-70%, 70%-90%, or 50%-90%.

In some embodiments, the circular probe or circularizable probe or probe set comprises 1, 2, 3, 4, 5, 6, or more modified nucleotide or nucleotide analog residues. Examples of suitable modified nucleotide or nucleotide analog residues include sugar modified nucleotides, such as 2′-O-methyl ribonucleic acid (2′-OMeRNA), locked nucleic acid (LNA) nucleotides, 2′-fluoro ribonucleic acid (2′-F RNA), or any combination thereof. In some embodiments, the modified nucleotide or nucleotide analog residues comprise one or more of: a sugar modified nucleotide, a C2′ modified nucleotide, a nucleotide with a C3′ endo pucker conformation, a modified ribonucleotide residue, a modified deoxyribonucleotide residue, a modified nucleotide or nucleotide analog residue comprising a triazole or thiol-based linkage instead of a phosphodiester linkage, and/or a modified nucleotide comprising a thiophosphate linkage. In some embodiments, the modified nucleotide or nucleotide analog residues comprise any suitable modification, (e.g. sugar, base, and/or linkage modification), such as any described herein, or in Ochoa and Milam, Molecules, 25(20):4659 (2020); and McKenzie et al., Chem Soc Rev., 50(8):5126-5164 (2021), the entire contents of each of which are incorporated herein by reference.

In some embodiments, the circular probe, circularizable probe or probe set, or circularized probe, is primarily composed of deoxyribonucleotide residues. In some embodiments, a probe primarily composed of deoxyribonucleotide residues, more than 50% of the nucleotide residues are deoxyribonucleotide residues.

FIG. 1C depicts the structure of an exemplary modified nucleotide or nucleotide analog residue that can decrease the rate of polymerization of a polymerase on a circular or circularized probe comprising said modified nucleotide or nucleotide analog residue (2′-OMeRNA). In some embodiments, the inclusion of a single 2′-OMeRNA residue in the template (e.g., in the circular or circularized probe) decreases the rate of polymerization by at least any of 20%, 30%, 40%, 50%, 60%, 70%, 80%, or 90% compared to the rate of polymerization on a corresponding reference template.

In some embodiments, the circular probe or circularized probe may comprise no more than any one of 20%, 15%, 10%, 5%, 2%, or 1% modified nucleotide or nucleotide analog residues and/or modified or unmodified ribonucleotide residues out of the total number of residues in the circular probe or circularized probe. In some embodiments, the circular probe or circularized probe may comprise at least any one of 1%, 2%, 3%, 4%, 5%, or 10% modified nucleotide or nucleotide analog residues and/or modified or unmodified ribonucleotide residues out of the total number of residues in the circular probe or circularized probe. In some embodiments, the circular probe or circularized probe may comprise 1%-20%, 1%-15%, 1%-10%, 1%-5%, 5%-10%, 5%-20%, or 10%-20% modified nucleotide or nucleotide analog residues and/or modified or unmodified ribonucleotide residues out of the total number of residues in the circular probe or circularized probe.

In some embodiments, the one or more modified nucleotide or nucleotide analog residues can comprise one or more backbone modified nucleotide or nucleotide analog residues. For example, the one or more modified nucleotide or nucleotide analog residues can comprise one or more thiol linkages and/or one or more triazole linkages. An exemplary structure of a triazole linkage is shown in FIG. 1D. In some embodiments, the thiol and/or triazole linkage is an internal linkage (e.g., is not formed at the ligation point to generate a circularized probe from the circularizable probe or probe set). In some embodiments, method comprises contacting the biological sample with the circular probe or circularizable probe or probe set comprising one, two, or more thiol and/or triazole linkages. In some embodiments, the circular probe or circularizable probe or probe set comprises two or more thiol and/or triazole linkages. In some embodiments, at least two of the two or more thiol and/or triazole linkages are separated by fewer than 100 nucleotide (nt) residues. In some embodiments, at least two of the two or more thiol and/or triazole linkages are separated by fewer than 90 nt residues, fewer than 80 nt residues, fewer than 70 nt residues, fewer than 60 nt residues, fewer than 50 nt residues, fewer than 40 nt residues, fewer than 30 nt residues, fewer than 20 nt residues, fewer than 10 nt residues, or fewer than 5 nt residues. In some embodiments, the one or more thiol linkages comprise one or more phosphorothioate and/or thiophosphate linkage(s). In some embodiments, the one or more backbone modified nucleotide or nucleotide analog residues comprise any of the linkages and/or modifications described herein, or in Ochoa and Milam, Molecules, 25(20):4659 (2020); and McKenzie et al., Chem Soc Rev., 50(8):5126-5164 (2021), the entire contents of each of which are incorporated herein by reference.

In some embodiments, the one or more modified nucleotide or nucleotide analog residues can include one or more nucleotide residue comprising a base other than A, T, G, C, or U. In some embodiments, the modified nucleotide or nucleotide analog residue comprises a universal or semi-universal base. In some embodiments, the universal base is 5-Nitroindole. In some embodiments, 5-Nitroindole directs random incorporation of nucleotides or incorporable nucleotide analogs in the RCA product. In some embodiments, the semi-universal base is deoxyinosine. Deoxyinosine can direct partially random incorporation of nucleotides or incorporable nucleotide analogs in the RCA product (e.g., with some bias toward incorporation of cytosine nucleotides or nucleotide analogs. In some embodiments, the universal base(s) and/or semi-universal base(s) are located outside of a barcode sequence or target hybridization sequence comprised by the circular or circularizable probe or probe set.

In some embodiments, the one or more modified nucleotide or nucleotide analog residues (e.g., 5-Nitroindole) decrease the processivity of the polymerase. Processivity is the ability of DNA polymerase to carry out continuous DNA synthesis on a template DNA without frequent dissociation. It can be measured by the average number of nucleotides incorporated by a DNA polymerase on a single association/disassociation event. Thus, in some aspects, the rate of polymerization of the polymerase using the circular or circularized probe as template comprising the one or more modified nucleotide or nucleotide analog residues is decreased by increasing the number or frequency of association/dissociation events. The modified nucleotide or nucleotide analog residues can act as “speed bumps”, slowing down polymerization by the polymerase on the template for RCA.

In some embodiments, a circular probe or circularizable probe or probe set hybridizes to a target nucleic acid in the biological sample. In some embodiments, the target nucleic acid is an endogenous analyte or is associated with an endogenous analyte (e.g., is a labelling agent or a component thereof), as described in Section II above. In some embodiments, hybridizing to the target nucleic acid in the biological sample comprises the pairing of substantially complementary or complementary nucleic acid sequences within two different molecules or sets of molecules, one of which is the endogenous analyte or the labelling agent (e.g., reporter oligonucleotide attached thereto) and one of which is the circular probe or circularizable probe or probe set. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, at least 90%, or at least 95%) of their individual bases are complementary to one another.

In some embodiments, a circularizable probe or probe set provided herein is capable of DNA-templated ligation, such as from a cDNA molecule. See, e.g., U.S. Pat. No. 8,551,710, which is hereby incorporated by reference in its entirety. In some embodiments, provided herein is a circularizable probe or probe set capable of RNA-templated ligation. See, e.g., U.S. Pat. Pub. 2020/0224244 which is hereby incorporated by reference in its entirety. In some embodiments, the circularizable probe set is a SNAIL probe set. See, e.g., U.S. Pat. Pub. 20190055594, which is hereby incorporated by reference in its entirety. In some embodiments, provided herein is a multiplexed proximity ligation assay. See, e.g., U.S. Pat. Pub. 20140194311 which is hereby incorporated by reference in its entirety. In some embodiments, provided herein is a circularizable probe or probe set capable of proximity ligation, for instance a proximity ligation assay for RNA (e.g., PLAYR) probe set. See, e.g., U.S. Pat. Pub. 20160108458, which is hereby incorporated by reference in its entirety. In some embodiments, a circular probe can be indirectly hybridized to the target nucleic acid. In some embodiments, the circular construct is formed from a circularizable probe set capable of proximity ligation, for instance a proximity ligation in situ hybridization (PLISH) probe set. See, e.g., U.S. Pat. Pub. 2020/0224243 which is hereby incorporated by reference in its entirety. In some embodiments, a target nucleic acid for a circularizable probe or probe set provided herein is a probe (such as an L-shaped probe) hybridized to an analyte in the sample, such as an mRNA molecule, such as in a RollFISH assay. See, e.g., Wu et al. Commun Biol 1, 209 (2018), which is hereby incorporated by reference in its entirety.

Any suitable circularizable probe or probe set, or indeed more generally circularizable reporter molecules, may be used to generate the RCA template which is used to generate the RCA product. Any of the circularizable probes or probe sets described herein can be modified to comprise one or more modified nucleotide or nucleotide analog residues as described herein, in order to decrease the rate of polymerization by a polymerase using a circularized probe generated therefrom as a template. By “circularizable” it is meant that the probe or reporter (the RCA template) is in the form of a linear molecule having ligatable ends which may circularized by ligating the ends together directly or indirectly, e.g. to each other, or to the respective ends of an intervening (“gap”) oligonucleotide or to an extended 3′ end of the circularizable RCA template. A circularizable probe or template may also be provided in two or more parts, namely two or more molecules (e.g. oligonucleotides) which may be ligated together to form a circle. When said probe or RCA template is circularizable it is circularized by ligation prior to RCA. Ligation may be templated using a ligation template, and in the case of circularizable probes such as padlock and molecular inversion probes, the target analyte may provide the ligation template, or the ligation template may be separately provided. The circularizable probe or RCA template (or part or portion thereof) will comprise at its respective 3′ and 5′ ends regions of complementarity to corresponding cognate complementary regions (or binding sites) in the ligation template, which may be adjacent where the ends are directly ligated to each other, or non-adjacent, with an intervening “gap” sequence, where indirect ligation is to take place.

In the case of circularizable probes such as padlock probes, in one embodiment, the ends of the probe may be brought into proximity to each other by hybridization to adjacent sequences on a target nucleic acid molecule (such as a target analyte), which acts as a ligation template, thus allowing the ends to be ligated together to form a circular nucleic acid molecule, allowing the circularized probe to act as a template for an RCA reaction. In such an example the terminal sequences of the probe which hybridize to the target nucleic acid molecule will be specific to the target analyte in question, and will be replicated repeatedly in the RCA product. They may therefore act as a marker sequence indicative of that target analyte. Accordingly, it can be seen that the marker sequence in the RCA product may be equivalent to a sequence present in the target analyte itself. Alternatively, a marker sequence (e.g. tag or barcode sequence) may be provided in the non-target complementary parts of the probe. In still a further embodiment, the marker sequence may be present in the gap oligonucleotide which is hybridized between the respective hybridized ends of the probe, where they are hybridized to non-adjacent sequences in the target molecule. Such gap-filling probes are akin to molecular inversion probes.

In some embodiments, similar circularized probes suitable as RCA template molecules can be generated using molecular inversion probes (a type of circularizable probe). Like padlock probes, these are also typically linear nucleic acid molecules capable of hybridizing to a target nucleic acid molecule (such as a target analyte) and being circularized. The two ends of the molecular inversion probe may hybridize to the target nucleic acid molecule at sites which are proximate but not directly adjacent to each other, resulting in a gap between the two ends. The size of this gap may range from only a single nucleotide in some embodiments, to larger gaps of 100 to 500 nucleotides, or longer, in other embodiments. Accordingly, it is necessary to supply a polymerase and a source of nucleotides (or nucleotide analogs), or an additional gap-filling oligonucleotide, in order to fill the gap between the two ends of the molecular inversion probe, such that it can be circularized. As with the padlock probe, the terminal sequences of the molecular inversion probe which hybridize to the target nucleic acid molecule, and the sequence between them, will be specific to the target analyte in question, and will be replicated repeatedly in the RCA product. They may therefore act as a marker sequence indicative of that target analyte. Alternatively, a marker sequence (e.g. tag or barcode sequence) may be provided in the non-target complementary parts of the molecular inversion probe.

In some embodiments, a circularized probe comprising modified nucleotide(s) and/or nucleotide analog(s) can be generated by incorporating the modified nucleotide(s) and/or nucleotide analog(s) during gap filling of a circularizable probe.

In some embodiments, the probes disclosed herein may be invader probes, e.g., for generating a circular nucleic acid such as a circularized probe. Such probes are of particular utility in the detection of single nucleotide polymorphisms. The methods of analyzing a biological sample provided by the present disclosure may, therefore, be used in the detection of a single nucleotide polymorphism, or indeed any variant base, in the target nucleic acid sequence. Probes for use in such a method may be designed such that the 3′ ligatable end of the circularizable probe or probe part of the probe set is complementary to and capable of hybridizing to the nucleotide in the target molecule which is of interest (the variant nucleotide), and the nucleotide at the 3′ end of the 5′ additional sequence at the 5′ end of the circularizable probe or at the 5′ end of another, different, probe part of the circularizable probe set is complementary to the same said nucleotide, but is prevented from hybridizing thereto by a 3′ ligatable end (e.g., it is a displaced nucleotide). Cleavage of the probe to remove the additional sequence provides a 5′ ligatable end, which may be ligated to the 3′ ligatable end of the probe or probe part if the 3′ ligatable end is hybridized correctly to (e.g. is complementary to) the target nucleic acid molecule. Probes designed according to this principle provide a high degree of discrimination between different variants at the position of interest, as only probes in which the 3′ ligatable end is complementary to the nucleotide at the position of interest may participate in a ligation reaction. In one embodiment, the probe is provided in a single part, and the 3′ and 5′ ligatable ends are provided by the same probe. In some embodiments, an invader probe is a padlock probe (an invader padlock or “iLock”), e.g., as described in Krzywkowski et al., Nucleic Acids Research 45, e161, 2017 and US 2020/0224244, which are incorporated herein by reference in their entirety.

In some embodiments, the ligation involves chemical ligation. In some embodiments, the ligation involves template dependent ligation. In some embodiments, the ligation involves template independent ligation. In some embodiments, the ligation involves enzymatic ligation.

In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity. In some embodiments, the ligase has RNA-templated DNA ligase activity and/or an RNA-templated RNA ligase activity. In some embodiments, the ligase is Chlorella virus DNA ligase (also known as PBCV-1 DNA ligase), a T4 RNA ligase, a T4 DNA ligase, or a single-stranded DNA (ssDNA) ligase. In some embodiments, the ligase is a PBCV-1 DNA ligase or variant or derivative thereof and/or a T4 RNA ligase 2 (also known as T4 Rnl2) or variant or derivative thereof.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, e.g., separated by one or more intervening nucleotides or “gaps”. In some embodiments, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, circularizable probe such as a padlock probe, or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, a probe disclosed herein (e.g., a circularizable probe or probe set) can comprise a 5′ flap which may be recognized by a structure-specific cleavage enzyme, e.g. an enzyme capable of recognizing the junction between single-stranded 5′ overhang and a DNA duplex, and cleaving the single-stranded overhang. It will be understood that the branched three-strand structure which is the substrate for the structure-specific cleavage enzyme may be formed by 5′ end of one probe part and the 3′ end of another probe part when both have hybridized to the target nucleic acid molecule, as well as by the 5′ and 3′ ends of a one-part probe. Enzymes suitable for such cleavage include Flap endonucleases (FENS), which are a class of enzymes having endonucleolytic activity and being capable of catalyzing the hydrolytic cleavage of the phosphodiester bond at the junction of single- and double-stranded DNA. Thus, in some embodiment, cleavage of the additional sequence 5′ to the first target-specific binding site is performed by a structure-specific cleavage enzyme, e.g. a Flap endonuclease. Suitable Flap endonucleases are described, for example, in Ma et al. 2000. JBC 275, 24693-24700 and in US 2020/0224244, the entire contents of each of which are incorporated herein by reference, and may include P. furiosus (Pfu), A. fulgidus (Afu), M. jannaschii (Mja) or M. thermoautotrophicum (Mth). In other embodiments an enzyme capable of recognizing and degrading a single-stranded oligonucleotide having a free 5′ end may be used to cleave an additional sequence (5′ flap) from a structure as described above. Thus, an enzyme having 5′ nuclease activity may be used to cleave a 5′ additional sequence. Such 5′ nuclease activity may be 5′ exonuclease and/or 5′ endonuclease activity. A 5′ nuclease enzyme is capable of recognizing a free 5′ end of a single-stranded oligonucleotide and degrading said single-stranded oligonucleotide. A 5′ exonuclease degrades a single-stranded oligonucleotide having a free 5′ end by degrading the oligonucleotide into constituent mononucleotides from its 5′ end. A 5′ endonuclease activity may cleave the 5′ flap sequence internally at one or more nucleotides. Further, a 5′ nuclease activity may take place by the enzyme traversing the single-stranded oligonucleotide to a region of duplex once it has recognized the free 5′ end, and cleaving the single-stranded region into larger constituent nucleotides (e.g. dinucleotides or trinucleotides), or cleaving the entire 5′ single-stranded region, e.g. as described in Lyamichev et al. 1999. PNAS 96, 6143-6148, the content of which is herein incorporated by reference in its entirety, for Taq DNA polymerase and the 5′ nuclease thereof. Preferred enzymes having 5′ nuclease activity include Exonuclease VIII, or a native or recombinant DNA polymerase enzyme from Thermus aquaticus (Taq), Thermus thermophilus or Thermus flavus, or the nuclease domain therefrom.

In some embodiments, ligation of the polynucleotides produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the RCA template may comprise the target analyte, or a part thereof, where the target analyte is a nucleic acid, or it may be provided or generated as a proxy, or a marker, for the analyte. As noted above, any suitable assay can be used for the detection of numerous different analytes, which use a RCA-based detection system, e.g., where the signal is provided by generating a RCP from a circular RCA template which is provided or generated in the assay, and the RCP is detected to detect the analyte. The RCP may thus be regarded as a reporter which is detected to detect the target analyte. However, the RCA template may also be regarded as a reporter for the target analyte; the RCP is generated based on the RCA template, and comprises complementary copies of the RCA template. The RCA template determines the signal which is detected, and is thus indicative of the target analyte. As will be described in more detail below, the RCA template may be a probe, or a part or component of a probe, or may be generated from a probe, or it may be a component of a detection assay (e.g., a reagent in a detection assay), which is used as a reporter for the assay, or a part of a reporter, or signal-generation system. The RCA template used to generate the RCP may thus be a circular (e.g. circularized) reporter nucleic acid molecule, namely from any RCA-based detection assay which uses or generates a circular nucleic acid molecule as a reporter for the assay. Since the RCA template generates the RCP reporter, it may be viewed as part of the reporter system for the assay. In some embodiments, the RCA template can comprise one or more modified nucleotide or nucleotide analog residues as described above. In other embodiments, the RCA template does not comprise modified nucleotide or nucleotide analog residues.

### IV. Rolling Circle Amplification

In some embodiments, a probe disclosed herein is amplified through rolling circle amplification (RCA). In some embodiments, the circular or circularizable probes or probe sets, such as a padlock probe or a probe set that comprises a padlock probe, contain one or more barcodes or complements thereof.

In some embodiments, an RCA primer is added to prime the RCA, for example as shown in FIG. 1A. In some embodiments, the RCA primer is added after probe hybridization and/or circularization. In some embodiments, the amplification primer is added with the circular or circularizable probe or probe set. In some embodiments, the RCA primer is complementary to and hybridizes to the circular or circularized probe. In some embodiments, the RCA primer may also be complementary to the target nucleic acid, such as a portion of the target nucleic acid adjacent to the portion of the target nucleic acid that is hybridized by the circular or circularized probe, for example as shown in FIG. 1A. In some instances, the RCA primer may be complementary to both the target nucleic acid and the circularizable probe (e.g., a SNAIL probe). In some instances, the target nucleic acid is a probe (such as an L-shaped probe) that can also function as a primer (e.g., as in RollFISH). In some embodiments, a washing step is performed to remove any unbound probes, primers, etc. In some embodiments, the wash is a stringency wash. Washing steps can be performed at any point during the methods described herein to remove any of the components of the reaction, as desired. For example, washing steps may be used to remove unhybridized probes, non-specifically bound probes, probes that have not been ligated, or other components present in or contacting the reaction mixture and/or biological sample.

In some instances, upon addition of a DNA polymerase in the presence of appropriate dNTP precursors and other cofactors, the RCA primer is elongated by replication of multiple copies of the circular template. The amplification step can utilize isothermal amplification or non-isothermal amplification. In some embodiments, after the formation of the hybridization complex and any subsequent circularization (such as ligation of, e.g., a padlock probe) the circularized probe is rolling-circle amplified to generate an RCA product (e.g., amplicon) containing multiple copies of the complement of the circular template (e.g., the circular probe or circularized probe). In some embodiments, the RCA comprises a linear RCA. In some embodiments, the RCA comprises a branched RCA. In some embodiments, the RCA comprises a dendritic RCA. In some embodiments, the RCA comprises any combination of the foregoing.

Suitable examples of DNA polymerases that can be used include, but are not limited to: E. coli DNA polymerase I, Bsu DNA polymerase, Bst DNA polymerase, Taq DNA polymerase, VENT™ DNA polymerase, DEEPVENT™ DNA polymerase, LongAmp® Taq DNA polymerase, LongAmp® Hot Start Taq DNA polymerase, Crimson LongAmp® Taq DNA polymerase, Crimson Taq DNA polymerase, OneTaq® DNA polymerase, OneTaq® Quick-Load® DNA polymerase, Hemo KlenTaq® DNA polymerase, REDTaq® DNA polymerase, Phusion® DNA polymerase, Phusion® High-Fidelity DNA polymerase, Platinum Pfx DNA polymerase, AccuPrime Pfx DNA polymerase, Phi29 DNA polymerase, Klenow fragment, Pwo DNA polymerase, Pfu DNA polymerase, T4 DNA polymerase and T7 DNA polymerase enzymes.

In some embodiments, rolling circle amplification products are generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and a variant or derivative of any of the foregoing.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C. In some embodiments, the RCA is performed at a temperature between 18° C. and 30° C. In some embodiments, the RCA is performed at 30° C.

In some aspects, the polynucleotides and/or amplification product (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix. Exemplary modification and polymer matrix that can be employed in accordance with the provided embodiments comprise those described in, for example, US 2016/0024555, US 2018/0251833, and US 2017/0219465, the entire contents of each of which are incorporated herein by reference. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate a cDNA nanoball (e.g., amplicon) containing multiple copies of the cDNA. Techniques for rolling circle amplification (RCA) include linear RCA, branched RCA, dendritic RCA, and combinations thereof. See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 1 1:1095-1099, 2001; Schweitzer et al, Nature Biotech. 20:359-365, 2002; and U.S. Pat. Nos. 6,054,274, 6,291,187, 6,323,009, 6,344,329 and 6,368,801, the entire contents of each of which are incorporated herein by reference. Exemplary polymerases for use in RCA include DNA polymerase such phi29 ((φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

Following amplification, the sequence of the amplicon (e.g., RCA product) or a portion thereof, can be determined or otherwise analyzed, for example by using detectably labeled probes and imaging, as described in Section VI. The sequencing or analysis of the amplification products can comprise sequencing by hybridization, sequencing by ligation, and/or fluorescent in situ sequencing, and/or wherein the in situ hybridization comprises sequential fluorescent in situ hybridization.

A. Reaction Mixtures for Slowing RCA

As discussed in Section III above, in some aspects, the rate of RCA can be decreased by including one or more modified nucleotide or nucleotide analog residues in the RCA template (e.g., in a circular probe or circularizable probe or probe set that is used to generate a circularized probe). The one or more modified nucleotides in the circular or circularized probe can effectively function as “speed bumps” to slow down the polymerase and/or decrease processivity of the polymerase. Additionally or alternatively, the methods provided herein can comprise contacting the sample with a reaction mixture designed to decrease the rate of polymerization by the polymerase. In some embodiments, the reaction mixture comprises one or more nucleotides or analogs thereof. In some embodiments, the nucleotides or analogs thereof comprise a non-incorporable nucleotide or analog thereof that binds transiently to the polymerase but is not incorporated by the polymerase. In some embodiments, the nucleotides or analogs thereof comprise an incorporable nucleotide or analog thereof that is configured to be incorporated by the polymerase at a slower rate than a corresponding nucleoside triphosphate. In some embodiments, the nucleotide or analog can comprise any nucleotide or analog thereof, or any nucleotide modification, described herein, or in Ochoa and Milam, Molecules, 25(20):4659 (2020); and McKenzie et al., Chem Soc Rev., 50(8):5126-5164 (2021), the entire contents of each of which are incorporated herein by reference.

As disclosed herein, reducing the rate of polymerization in RCA, for example by contacting the sample with the reaction mixture comprising nucleotides or analogs thereof, may provide several advantages. For example, in some embodiments, an RCA rate can be altered so that there are few large signal spots (e.g., even for highly expressed genes) that overlap with one another and/or mask adjacent smaller signal spots, thus ameliorating the issues of optical crowding and allowing neighbouring spots to be distinguished and/or better resolve from one another, for instance as RCA product size and brightness become more homogeneous in the same microscope field of view. In some embodiments, the RCA rate can be adjusted such that there are few extremely bright signal spots, thus allowing relatively dim spots to be detected simultaneously with the bright spots. In some embodiments, adjusting the rate of polymerization in RCA, for example by contacting a sample with the reaction mixture, could allow for adjacent reactions to proceed at different rates while being carried out at the same temperature and/or for the same amount of time, thus, for example, reducing the complexity of the instrumentation and/or experimental setup. In such an example, in parallel reactions, a first reaction could include only unmodified dNTPs, and a second reaction could further include nucleotides or analogs thereof (e.g. a non-incorporable nucleotide, and/or incorporable nucleotide or analog thereof that is configured to be incorporated by the polymerase at a slower rate than a corresponding nucleoside triphosphate) that result in a reduced rate of polymerization in the RCA. In some embodiments, the rate of polymerization could be controlled based on the concentration of one or more of the nucleotides or analogs thereof for slowing polymerization.

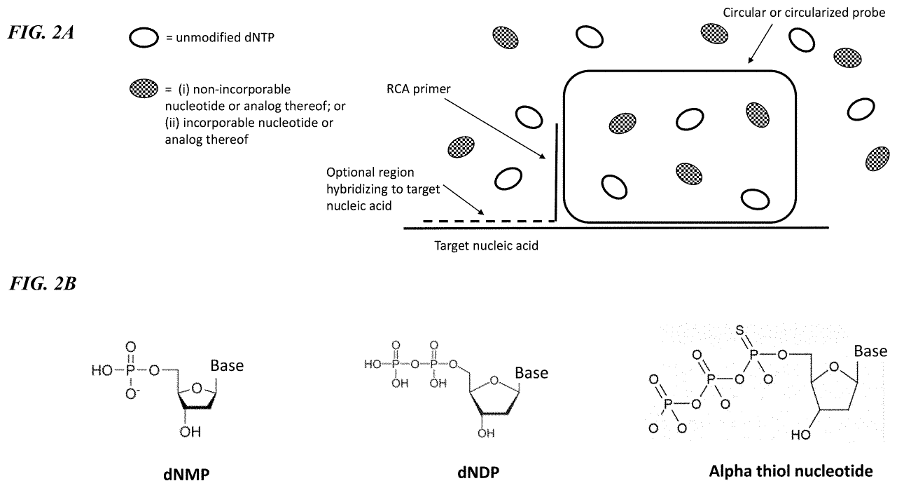

As shown in FIG. 2A, in some embodiments, a method for analyzing a biological sample can comprise contacting the biological sample with a reaction mixture comprising (i) a non-incorporable nucleotide or analog thereof that binds transiently to the polymerase but is not incorporated by the polymerase, and/or (ii) an incorporable nucleotide or analog thereof that is configured to be incorporated by the polymerase at a slower rate than a corresponding nucleoside triphosphate. Optionally, the reaction mixture additionally comprises unmodified dNTPs, as shown.

In some embodiments, a non-incorporable nucleotide or analog thereof is a nucleotide or nucleotide analog that is not incorporable or is substantially non-incorporable by a polymerase. In some embodiments, the non-incorporable nucleotide or analog thereof binds to the polymerase, but is not incorporated into the RCA product being generated by the polymerase. In some embodiments, the non-incorporable nucleotide or analog thereof is not incorporable by the polymerase. In some embodiments, the non-incorporable nucleotide or analog thereof binds transiently to the polymerase, meaning that it can be dissociated from the polymerase. Thus, binding of the non-incorporable nucleotide or analog thereof to the polymerase does not terminate the RCA reaction. In some embodiments, the non-incorporable nucleotide or analog thereof is a nucleotide monophosphate (e.g. deoxyribonucleoside monophosphate (dNMP)) or analog thereof, for example as shown in FIG. 2B. In some embodiments, the one or more non-incorporable nucleotides or analogs thereof comprise one or more of AMP, GMP, CMP, UMP, dAMP, dGMP, dCMP, and/or dTMP). In some embodiments, the non-incorporable nucleotide or analog thereof competes for binding to the polymerase with one or more NTPs and/or dNTPs, thus reducing the rate of polymerization.

In some embodiments, an incorporable nucleotide or analog thereof that is configured to be incorporated by the polymerase at a slower rate than a corresponding nucleoside triphosphate is a nucleotide or nucleotide analog other than an unmodified NTP/dNTP. Changes to nucleotide configuration can affect the rate of incorporation by a polymerase. For example, rate-limiting steps such as NTP or dNTP recognition can be sensitive to both nucleobase and backbone modifications. In some embodiments, the incorporable nucleotide or analog thereof is configured to be incorporated by the polymerase at a rate that is less than any one of 80%, 75%, 70%, 65%, 60%, 55%, 50%, 45%, 40%, 35%, or 30% of the rate of incorporation for a corresponding nucleoside triphosphate. In some embodiments, the incorporable nucleotide or analog thereof is configured to be incorporated by the polymerase at a rate that is at least any one of 5%, 10%, 20%, 30%, 35%, 40%, 45%, 50%, 60%, 65%, or 70% of the rate of incorporation for a corresponding nucleoside triphosphate. In some embodiments, the incorporable nucleotide or analog thereof is configured to be incorporated by the polymerase at a rate that any one of 5%-10%, 5%-20%, 5%-30%, 5%-80%, 10%-80%, 10%-60%, 10%-50%, 10%-20%, 20%-30%, 20%-80%, 40%-80%, 50%-80%, 40%-50%, 50%-60%, 60%-70%, or 70%-80% of the rate of incorporation for a corresponding nucleoside triphosphate.

In some embodiments, the incorporable nucleotide or analog thereof that is configured to be incorporated by the polymerase at a slower rate than a corresponding nucleoside triphosphate corresponds to any one of the following nucleoside triphosphates: dATP, dCTP, dGTP, dTTP, ATP, GTP, CTP, and UTP. In general, the incorporable nucleotide or analog thereof corresponds to a nucleoside triphosphate with the most similar structure, and/or the same or similar nucleobase. For example, in some embodiments, alpha-thio-dATP (an alpha-thio derivative of dATP) corresponds to dATP. Similarly, in some embodiments, alpha-thio-dTTP corresponds to dTTP, alpha-thio-dCTP corresponds to dCTP, alpha-thio-GTP corresponds to dGTP, and so on. It can be seen that in some aspects, a corresponding nucleoside triphosphate will be readily recognized for a given incorporable nucleotide or analog thereof described herein. In some embodiments, the corresponding nucleoside triphosphate is naturally-occurring.

In some embodiments, the reaction mixture comprises a ratio of one or more incorporable nucleotide or analog thereof (e.g., α-thio-dNTPs) to unmodified dNTPs of at least any one of 1:1000, 1:100, 1:50, 1:20, 1:10, 1:5, 1:2, 1:1, 2:1, 5:1, 10:1, 100:1, or 1000:1. In some embodiments, the reaction mixture comprises one or more incorporable nucleotides or analogs thereof, and does not comprise any unmodified dNTPs. In some embodiments, the reaction mixture comprises a ratio of one or more incorporable nucleotide or analog thereof (e.g., α-thio-dNTPs) to unmodified dNTPs of no more than any one of 1:1000, 1:100, 1:50, 1:20, 1:10, 1:5, 1:2, 1:1, 2:1, 5:1, 10:1, 100:1, or 1000:1. In some embodiments, the reaction mixture comprises a ratio of one or more incorporable nucleotide or analog thereof (e.g., α-thio-dNTPs) to unmodified dNTPs of no more than any one of 1:1000-1:100, 1:1000-1:50, 1:1000-1:20, 1:1000-1:1, 1:2-100:1, 1:2-1000:1, or 1:10-10:1.

In some embodiments, the reaction mixture comprises a ratio of one or more non-incorporable nucleotide or analog thereof (e.g., NMPs/dNMPs) to unmodified dNTPs of at least any one of 1:1000, 1:100, 1:50, 1:20, 1:10, 1:5, 1:2, 1:1, 2:1, 5:1, 10:1, or 100:1. In some embodiments, the reaction mixture comprises a ratio of one or more non-incorporable nucleotide or analog thereof (e.g, NMPs/dNMPs) to unmodified dNTPs of no more than any one of 1:1000, 1:100, 1:50, 1:20, 1:10, 1:5, 1:2, 1:1, 2:1, 5:1, 10:1, 100:1, or 1000:1. In some embodiments, the reaction mixture comprises a ratio of one or more non-incorporable nucleotide or analog thereof (e.g, NMPs/dNMPs) to unmodified dNTPs of no more than any one of 1:1000-1:100, 1:1000-1:50, 1:1000-1:20, 1:1000-1:1, 1:2-100:1, 1:2-1000:1, or 1:10-10:1.

In some embodiments, nucleoside diphosphates (e.g., ADP, GDP, CDP, UDP, dADP, dGDP, dCDP, and/or dUDP) are included in the reaction mixture. In some embodiments, a method provided herein comprises contacting the sample with a reaction mixture comprising one or more incorporable nucleotides other than unmodified dNTPs that are configured to be incorporated by the polymerase at a slower rate than unmodified dNTPs. In some embodiments, the incorporable nucleotides can be NDPs/dNDPs. In some embodiments, NDPs/dNDPs can be incorporated by the polymerase at a slower rate than unmodified NDPs/dNDPs (e.g., as described in Cassandra et al. PNAS 2018, 115 (5) 980-985, Varela et al. Biochemistry 2021, 60 (5) 373-380, Garforth et al. PLoS One. 2008, 3(4):e2074, the contents of each of which are herein incorporated by reference in their entirety). For example, NDPs/dNDPs may be incorporated at a rate that is at least 10, at least 15, at least 20, or at least 30 fold slower than the rate of incorporation of corresponding unmodified dNTPs. Slowing the rate of polymerization by the polymerase by including NDPs/dNDPs in the reaction mixture does not require incorporation of the NDPs/dNDPs into the RCA product, however. In some cases, the NDPs/dNDPs bind transiently to the polymerase and dissociate without being incorporated. Thus, NDPs/dNDPs can slow the rate of polymerization by competing with other nucleotides (such as dNTPs) for binding to the polymerase. In some embodiments, the reaction mixture comprises one or more dNDPs selected from dADP, dGDP, dCDP, dTDP, and combinations thereof. In some embodiments, the reaction mixture comprises one or more NDPs selected from ADP, GDP, CDP, UDP, and combinations thereof. In some embodiments, the reaction mixture comprises NDPs/dNDPs and dNTPs. In some embodiments, the rate of polymerization by the polymerase is inversely correlated with the concentration of NDPs and/or dNDPs in the reaction mixture and/or with the ratio of dNDPs to dNTPs in the reaction mixture.

In another example of an incorporable nucleotide or analog thereof configured to be incorporated by the polymerase at a slower rate than a nucleoside triphosphate (e.g., dATP, dTTP, dUTP, dCTP, or dUTP), an incorporable alpha thiol nucleotide analog can be included in the reaction mixture. The structure of an alpha thiol nucleotide, comprising a sulfur in place of an oxygen at the alpha phosphate position, is shown in FIG. 2B. The effect of alpha thiol dNTPs on Klenow fragment polymerization rates have been described. See, for example, Pugliese et al. J Am Chem Soc. 2015; 137(30):9587-9594, the content of which is herein incorporated by reference in its entirety. In some embodiments, the alpha thiol nucleotide is configured to be incorporated by the polymerase at a rate that is less than any one of 80%, 75%, 70%, 65%, 60%, 55%, 50%, 45%, 40%, 35%, or 30% of the rate of incorporation for a corresponding nucleoside triphosphate. In some embodiments, the alpha thiol nucleotide is configured to be incorporated by the polymerase at a rate that is at least any one of 5%, 10%, 20%, 30%, 35%, 40%, 45%, 50%, 60%, 65%, or 70% of the rate of incorporation for a corresponding nucleoside triphosphate. In some embodiments, the alpha thiol nucleotide is configured to be incorporated by the polymerase at a rate that any one of 5%-10%, 5%-20%, 5%-30%, 5%-80%, 10%-80%, 10%-60%, 10%-50%, 10%-20%, 20%-30%, 20%-80%, 40%-80%, 50%-80%, 40%-50%, 50%-60%, 60%-70%, or 70%-80% of the rate of incorporation for a corresponding nucleoside triphosphate. A corresponding nucleoside triphosphate is a dNTP having the same base as the alpha thiol (e.g. α-thio-dATP corresponds to dATP, α-thio-dGTP corresponds to dGTP, and so on).

In some embodiments, the alpha thiol nucleotide increases the amount of time the polymerase spends in the open conformation (τₒₚₑₙ) when processing the alpha thiol nucleotide compared to a corresponding unmodified nucleoside triphosphate. In some embodiments, the alpha thiol nucleotide is configured to increase τₒₚₑₙ by at least any one of 1.2-, 1.5-, 2-, 2.5-, or 3-fold compared to τₒₚₑₙ for an unmodified nucleoside triphosphate. In some embodiments, the mean open time of the polymerase during polymerization for incorporation of the incorporable nucleotide (e.g., an alpha thiol nucleotide) is at least 125%, 150%, 200%, 225%, or 250% of the mean open time for incorporation of a corresponding nucleoside triphosphate.

In some embodiments, the one or more incorporable nucleotides or analogues thereof comprise a gamma thiol modified nucleotide. In some embodiments, the gamma thiol modified nucleotide is selected from γ-thio-dATP, γ-thio-dGTP, γ-thio-dTTP, and γ-thio-dCTP. In some embodiments, the gamma thiol modified nucleotide is γ-thio-dATP.

In some embodiments, the one or more incorporable nucleotides or analogs thereof configured to be incorporated by the polymerase at a slower rate than a nucleoside triphosphate comprise one or more nucleotides with modified bases. In some examples, one or more unmodified dNTPs selected from dATP, dGTP, dTTP, and dCTP are substituted with at least 25%, at least 30%, at least 40%, at least 50%, at least 60%, at least 70%, at least 80%, at least 90%, or 100% of a corresponding modified nucleotide or analogue thereof. In some embodiments, one or more unmodified dNTPs selected from dATP, dGTP, dTTP, and dCTP are substituted with no more than 90%, no more than 80%, no more than 75%, no more than 60%, no more than 50%, no more than 40%, or no more than 30% of a corresponding modified nucleotide or analogue thereof. Any combination of these range end points can also be used, such as between 25% and 75%, between 30% and 60%, or between 70% and 90% substitution, for example. In some embodiments, multiple unmodified dNTPs selected from dATP, dGTP, dTTP, and dCTP are partially or completely substituted by modified nucleotides or analogs thereof in the reaction mixture. In some embodiments, the effects of substituting multiple different nucleotides with modified nucleotides are additive.

In some embodiments, the one or more incorporable nucleotides or analogs thereof comprise one or more azide modified nucleotides and/or one or more alkyne modified nucleotides. In some embodiments, the reaction mixture comprises at least 25%, at least 30%, at least 40%, at least 50%, at least 60%, at least 70%, at least 80%, at least 90%, or 100% of an azide modified dNTP substituted for a corresponding unmodified dNTP. In some embodiments, the reaction mixture comprises no more than 90%, no more than 80%, no more than 75%, no more than 60%, no more than 50%, no more than 40%, or no more than 30% of an azide modified dNTP substituted for a corresponding unmodified dNTP. Exemplary azide modified dNTPs include but are not limited to 5-Azidomethyl-dUTP, Azide-PEG₄-Aminoallyl-dUTP, and 5-Azido-PEG₄-dCTP. In some embodiments, the reaction mixture comprises at least 25%, at least 30%, at least 40%, at least 50%, at least 60%, at least 70%, at least 80%, at least 90%, or 100% of an alkyne modified dNTP substituted for a corresponding unmodified dNTP. In some embodiments, the reaction mixture comprises no more than 90%, no more than 80%, no more than 75%, no more than 60%, no more than 50%, no more than 40%, or no more than 30% of an alkyne modified dNTP substituted for a corresponding unmodified dNTP. Exemplary alkyne modified nucleotides include, but are not limited to, 5-Ethynyl-dUTP and C8-Alkyne-dCTP. In some embodiments, a method described herein comprises incorporating one or more azide and/or alkyne modified dNTPs into the RCA product, wherein the method does not comprise performing a click reaction or a copper-catalyzed reaction.

In some embodiments, the one or more incorporable nucleotides or analogs thereof comprise one or more modified nucleotides comprising base modifications. In addition to the exemplary azide and/or alkyne base modifications described above, exemplary base modifications can include dibenzylcyclooctyl (DBCO) modifications, vinyl modifications, trans-Cyclooctene (TCO), and so on. In some embodiments, wherein the nucleotide modification comprises a functional group suitable for performing a click reaction, the method does not comprise performing a click reaction on the biological sample prior to imaging to detect an RCA product.

In some aspects, the reaction mixture does not comprise an amine-modified nucleotide. In some aspects, the reaction mixture does not comprise a nucleotide modified with a fluorophore. In some aspects, the reaction mixture does not comprise amine-modified nucleotides or fluorescently modified nucleotides. In some aspects, the amplification product (e.g., RCA product) does not comprise amine-modified nucleotide or fluorescently modified nucleotide or nucleotide analog residues. In other embodiments, the one or more incorporable nucleotides or analogs thereof can include one or more amine modified nucleotides. In some embodiments, the one or more incorporable nucleotides or analogs thereof can be functionalized, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. Examples of other amine-modified nucleotides comprise, but are not limited to, a 5-Aminoallyl-dUTP moiety modification, a 5-Propargylamino-dCTP moiety modification, a N⁶-6-Aminohexyl-dATP moiety modification, or a 7-Deaza-7-Propargylamino-dATP moiety modification. In some embodiments, the modified nucleotide comprises an acrylic acid or N-hydroxysuccinimide (NHS) moiety modification.

In some embodiments, the mean rate of polymerization by the polymerase in the rolling circle amplification is less than 2280 nt/min, less than 2000 nt/min, less than 1500 nt/min, less than 1250 nt/min, less than 1000 nt/min, less than 750 nt/min, less than 500 nt/min, or less than 250 nt/min, optionally wherein the rate of polymerization is measured at 30° C. In some embodiments, the mean rate of polymerization by the polymerase in the rolling circle amplification is at least any one of 100, 200, 250, 500, 750, 1000, 1250, 1500, or 2000 nt/min, optionally wherein the rate of polymerization is measured at 30° C. In some embodiments, the mean rate of polymerization by the polymerase is within any one of 100 nt/min-2280 nt/min, 100 nt/min-2000 nt/min, 100 nt/min-1500 nt/min, 200 nt/min-2280 nt/min, 200 nt/min-2000 nt/min, 200 nt/min-1500 nt/min, 200 nt/min-1000 nt/min, 500 nt/min-1000 nt/min, 1000 nt/min-1500 nt/min, 1250 nt/min-2000 nt/min, and 1500 nt/min-2000 nt/min.

In some embodiments, the RCA reactions for a plurality of circular templates in the sample can be terminated at the same time to provide a plurality of rolling circle amplification products. In some embodiments, the RCA reactions can be terminated by contacting the sample with a buffer comprising EDTA (e.g., Tris-EDTA or TE buffer).

B. Reaction Mixtures for Reducing Size of RCA Products

As discussed in Sections III and IV.A. above, provided herein are various methods of slowing down or reducing the rate of polymerization of an RCA product by a polymerase. In some aspects, slowing down the polymerization of an RCA product decreases the size of the RCA product. In some aspects, provided herein are methods for decreasing the size of the RCA product independently of a slowing effect on polymerization (e.g., by promoting compaction of the RCA product and/or base stacking interactions within the RCA product). In some aspects, reducing the size (e.g., mean diameter) of RCA products can reduce optical crowding. In some aspects, provided herein are methods that reduce the size (e.g., mean diameter) of RCA products without significantly reducing the mean intensity, mean signal-to-noise ratio, and/or mean density of the RCA products in a sample.

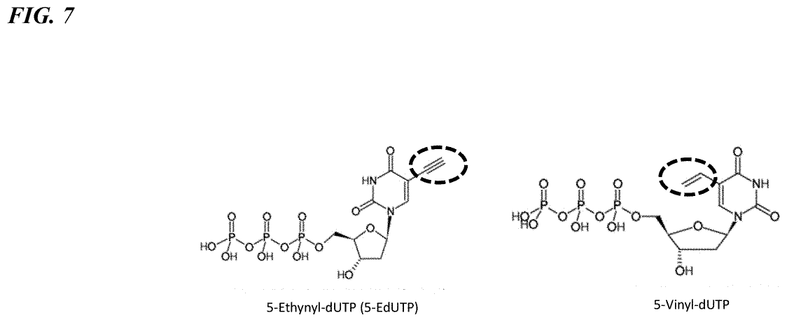

In some aspects, provided herein is a method for analyzing a biological sample, comprising: (a) contacting the biological sample with a reaction mixture comprising one or more nucleotide analogs, and (b) performing rolling circle amplification (RCA) of a circular nucleic acid template in the biological sample using a polymerase, thereby generating an RCA product incorporating the one or more nucleotide analogs, wherein the one or more nucleotide analogs comprise a nucleotide analog comprising a hydrophobic modification. In some embodiments, the hydrophobic modification is a base modification. In some embodiments, the hydrophobic modification comprises a carbon chain and/or a hydrocarbon ring. In some embodiments, the hydrophobic modification comprises a triple bond. In some embodiments, the hydrophobic modification comprises a vinyl or ethynyl group. Exemplary nucleotide analogs comprising hydrophobic modifications include but are not limited to ethynyl-dUTP or a vinyl-dUTP. As shown, for example, in FIG. 7, 5-ethynyl-dUTP (5-EdUTP) and 5-vinyl-dUTP include hydrophobic modifications (dashed circles in figure). In some embodiments, the nucleotide analog comprising the hydrophobic modification is a 5-ethynyl-dUTP or a 5-vinyl-dUTP. In some aspects, the incorporation of the one or more nucleotide analogs into the RCA product increases overall hydrophobicity of the RCA product. Example 5 provides data demonstrating a compaction effect of exemplary nucleotide analogs 5-ethynyl-dUTP or a 5-vinyl-dUTP incorporated into RCA products.

Without being bound by theory, in some aspects, the incorporation of the one or more nucleotide analogs into the RCA product promotes base stacking interactions among nucleotides in the RCA product, thereby promoting the RCA product to collapse into a smaller structure. In an aqueous environment, for example, the hydrophobic moieties of the RCA product (RCP) will be favorably positioned inwards toward the center of an RCP, making a hydrophobic core, while charged groups (e.g., phosphate groups) will tend to be situated on the outside of the RCP.

In some embodiments, the diameter of the RCA product generated using the one or more nucleotide analogs is smaller than a reference RCA product produced using the same template without including the one or more nucleotide analogs in the reaction mixture. In some embodiments, the median diameter of an RCA product generated using the one or more nucleotide analogs is smaller than the median diameter of a reference RCA product produced using the same template without including the one or more nucleotide analogs in the reaction mixture. In some embodiments, the median diameter of an RCA product generated using the one or more nucleotide analogs is less than 500 nm. In some embodiments, the median diameter of an RCA product generated without the nucleotide analogs (e.g., with only unmodified dNTPs) under the same conditions is at least 600 nm, at least 700 nm, or at least 800 nm. In some embodiments, the median diameter of an RCA product generated using the one or more nucleotide analogs is no more than 90% or no more than 80% of the median diameter of a reference RCA product produced using the same template without including the one or more nucleotide analogs in the reaction mixture. In some aspects, the mean diameter of the RCA products incorporating the one or more nucleotide analogs is reduced, but the average intensity, average signal-to-noise ratio, and/or density of the RCA products incorporating the one or more nucleotide analogs is not significantly different from the average intensity, average signal-to-noise ratio, and/or density of a reference RCA product produced using the same template without including the one or more nucleotide analogs in the reaction mixture. Any suitable method for determining the significance of differences in values can be used. For example, in some embodiments, a significant difference can be defined as a p-value of less than or equal to 0.05 using a two-tailed or one-tailed t-test. In some embodiments, the size (e.g., diameter) of one or more RCA products is determined by any suitable means, such as those described in Section III.

In some embodiments, the nucleotide analog comprising the hydrophobic modification is added to the sample at a concentration of at least 1 μM, at least 1.25 μM, at least 2.5 μM, at least 5 μM, at least 10 μM, at least 20 μM, at least 40 μM, at least 80 μM, or at least 100 μM. In some embodiments, the nucleotide analog comprising the hydrophobic modification is added to the sample at a concentration of no more than 1 mM, no more than 750 μM, no more than 500 μM, 200 μM, no more than 150 μM, no more than 100 μM, no more than 80 μM, no more than 60 μM, or no more than 40 μM. In some embodiments, the nucleotide analog comprising the hydrophobic modification is added to the sample at a concentration of between or between about any one of 1 μM and 200 μM, 1 μM and 150 μM, 1 μM and 100 μM, 1 μM and 80 μM, 10 μM and 200 μM, 10 μM and 150 μM, 10 μM and 100 μM, 10 μM and 80 μM, 50 μM and 200 μM, 50 μM and 100 μM, 50 μM and 80 μM, 60 μM and 200 μm, 60 μM and 100 μM, 60 μM and 80 μM, or 80 μM and 100 μM. In some embodiments, the nucleotide analog comprising the hydrophobic modification is added to the sample at a concentration of between about 60 μM and about 100 μM, or between about 80 μM and about 100 μM. In some embodiments, the nucleotide analog comprising the hydrophobic modification is added to the sample at a concentration of about 80 μM or at least about 80 μM. In some embodiments, the nucleotide analog comprising the hydrophobic modification is added to the sample at a concentration of at or about 1 μM, at or about 1.25 μM, at or about 2.5 μM, at or about 5 μM, at or about 10 μM, at or about 20 μM, at or about 40 μM, at or about 80 μM, or at or about 100 μM. In some embodiments, the nucleotide analog comprising the hydrophobic modification is added to the sample at a concentration of 100 μM.

In some embodiments, the nucleotide analog is a modified dUTP and the ratio of the modified dUTP to an unmodified dUTP or dTTP in the reaction mixture is between about 80:20 and about 1:99, optionally wherein the ratio of the modified dUTP to the unmodified dUTP or dTTP in the reaction mixture is between about 80:20 and about 40:60.

In some embodiments according to any of the methods described herein for slowing RCA and/or decreasing the size of RCA products, the plurality of RCA products can have a mean or median diameter of about 0.05 μm, about 0.1 μm, about 0.2 μm, about 0.3 μm, about 0.4 μm, about 0.5 μm, about 0.6 μm, about 0.7 μm, about 0.8 μm, about 0.9 μm, about 1.0 μm, about 1.1 μm, about 1.2 μm, about 1.3 μm, about 1.4 μm, or about 1.5 μm, or between any of the aforementioned values. In some embodiments, the plurality of rolling circle amplification products can have a mean or median diameter smaller than 0.25 μm.

In some embodiments, the plurality of RCA products can have a mean or median length of about 1 kb, about 2 kb, about 5 kb, about 10 kb, about 20 kb, about 30 kb, about 40 kb, about 50 kb, about 60 kb, or about 70 kb, or between any of the aforementioned values. In some embodiments, the plurality of rolling circle amplification products can have a mean or median length less than 20 kb or less than 10 kb.

In some embodiments, the mean or median number of copies of a unit sequence complementary to the circular nucleic acid in the plurality of RCA products can be about 10, about 50, about 100, about 500, about 1,000, about 5,000, or about 10,000 or more.

In some embodiments, the mean or median number of copies of a unit sequence complementary to the circular nucleic acid in the plurality of rolling circle amplification products can be less than 100 or less than 1,000.

In some embodiments, the polymerase extension (e.g., the RCA) may be performed for no more than 3 hours. In some embodiments, the polymerase extension may be performed for no more than 2 hours. In some embodiments, the polymerase extension may be performed for no more than 1 hour. In some embodiments, the polymerase extension may be performed for no more than 30 minutes. In any of the embodiments here, the polymerase extension, e.g., RCA, can be performed between about 20° C. and about 40° C., for instance, at about 25° C., about 26° C., about 27° C., about 28° C., about 29° C., about 30° C., about 31° C., about 32° C., about 33° C., about 34° C., about 35° C., about 36° C., or about 37° C., for less than about 5 minutes, less than about 10 minutes, less than about 15 minutes, less than about 20 minutes, less than about 25 minutes, less than about 30 minutes, less than about 35 minutes, less than about 40 minutes, less than about 45 minutes, less than about 50 minutes, less than about 55 minutes, less than about 60 minutes, less than about 65 minutes, less than about 70 minutes, less than about 75 minutes, less than about 80 minutes, less than about 85 minutes, less than about 90 minutes, less than about 95 minutes, less than about 100 minutes, less than about 105 minutes, less than about 110 minutes, less than about 115 minutes, less than about 120 minutes. In some embodiments, the polymerase extension can be performed for less than about 1 hour, less than about 2 hours, less than about 3 hours, less than about 4 hours, less than about 5 hours, less than about 6 hours, less than about 7 hours, less than about 8 hours, less than about 9 hours, less than about 10 hours, less than about 11 hours, less than about 12 hours, less than about 13 hours, less than about 14 hours, less than about 15 hours, less than about 16 hours, less than about 17 hours, less than about 18 hours, less than about 19 hours, less than about 20 hours, less than about 21 hours, less than about 22 hours, less than about 23 hours, less than about 24 hours, less than about 30 hours, less than about 35 hours, or less than about 40 hours. In a specific embodiment, the polymerase extension can be performed for between about 10 to 24 hours.

### V. Nucleotides, Nucleotide Analogs, and Modifications

In some aspects, provided herein are nucleotides, nucleotide analogs, and modified nucleotides, to be used in connection with any of the methods and compositions described herein.

In some aspects, a nucleotide, such as a naturally-occurring/unmodified nucleotide, comprises (i) a five-carbon sugar (e.g., sugar moiety or sugar ring), for example ribose for ribonucleotides or deoxyribose for deoxyribonucleotides, (ii) a nucleobase (e.g. adenine, cytosine, guanine, thymine, or uracil), and (iii) one or more phosphate groups. A nucleotide can exist as a monomer, such as a nucleoside triphosphate, that is not comprised by a larger nucleic acid molecule. In some aspects, a nucleotide can be a nucleotide residue in a nucleic acid molecule (e.g., a polynucleotide or oligonucleotide). In some aspects, in a nucleic acid molecule, multiple nucleotides (e.g., nucleotide residues) are covalently attached via a sugar-phosphate backbone, each nucleotide being attached to the next via a phosphodiester linkage. The structure of unmodified nucleotides, nucleotide residues, and nucleic acids has been described in detail, for example in Alberts et al, Molecular Biology of the Cell, Seventh Edition (W. W. Norton & Company, 2022).

In some embodiments, a modified nucleotide (e.g., modified nucleotide or nucleotide analog residue, or a nucleotide analog) is any suitable nucleotide that comprises a modification, for example as compared to an unmodified nucleotide, such as an unmodified deoxyribonucleotide or ribonucleotide. In some aspects, a modified nucleotide is a nucleotide analog. In some embodiments, a modification is a chemical and/or structural difference between the modified nucleotide and an unmodified nucleotide (e.g., a corresponding unmodified nucleotide). In some embodiments, a modified nucleotide comprises a modification to the nucleobase, the sugar moiety, the one or more phosphate groups, and/or the phosphodiester linkage (in the case of nucleotide residues). In some embodiments, a modified nucleotide comprises a modification that is present in nature. In some embodiments, a modified nucleotide comprises a modification that is not present in nature. In some embodiments, a modified nucleotide comprising modification(s) not present in nature is incorporated less efficiently and/or at a slower rate by a polymerase (which can be naturally occurring or modified by protein engineering) than a corresponding unmodified nucleotide (e.g., unmodified dATP, dTTP, dUTP, dCTP, or dGTP) that does not comprise the modification(s) in the modified nucleotide. In some embodiments, a modified nucleotide can comprise any suitable modification or combination thereof, such as any described herein, or any described, for example, in Ochoa and Milam, Molecules, 25(20):4659 (2020); and McKenzie et al., Chem Soc Rev., 50(8):5126-5164 (2021), the entire contents of each of which are incorporated herein by reference.

In some embodiments, a nucleoside triphosphate is deoxyadenosine triphosphate (dATP), deoxycytidine triphosphate (dCTP), deoxyguanosine triphosphate (dGTP), deoxythymidine triphosphate (dTTP), or deoxyuridine triphosphate (dUTP), adenosine triphosphate (ATP), guanosine triphosphate (GTP), cytidine triphosphate (CTP), thymidine triphosphate (TTP) and uridine triphosphate (UTP). In some embodiments, an unmodified nucleotide residue is a nucleotide residue resulting from incorporation of a nucleoside triphosphate by a polymerase into a nucleic acid, or a nucleotide residue having an identical structure to a nucleotide residue resulting from incorporation of a nucleoside triphosphate by a polymerase into a nucleic acid.

In some embodiments, a modified nucleotide comprises a modification to the sugar-phosphate backbone. In some embodiments, a backbone modified nucleotide, or modified backbone nucleotide, is a nucleotide, such as a nucleotide residue, comprising a sugar and/or phosphate modification, for example to the sugar phosphate backbone.

In some embodiments, a modified nucleotide is a sugar modified nucleotide, or modified sugar nucleotide. In some embodiments, a sugar modified nucleotide comprises a modification to the sugar moiety. In some aspects, a sugar modified nucleotide comprises a modification on a specific carbon atom of the sugar moiety, and is designated as such. In some aspects, carbon atoms of the sugar moiety of a nucleotide are numbered 1 through 5 (e.g., C1′, C2′, C3′, C4′, C5′). Thus, in some embodiments, a C2′ modified nucleotide is a sugar modified nucleotide with a modification on the C2′ carbon of the sugar moiety; a C3′ modified nucleotide is a sugar modified nucleotide with a modification on the C3′ carbon of the sugar moiety; and so on. For example, the C2′ carbon can be attached to fluorine, for example as in the case of 2′-fluoro ribonucleic acid (2′-F RNA). Alternatively, the C2′ carbon can be attached to a methoxy/OMe (—OCH₃) group, for example in the case of 2′-O-methyl ribonucleic acid (2′-OMeRNA). Other exemplary C2′ modifications include an amino (—NH₂) group or azido (—N₃) group attached to the C2′ carbon.

In some embodiments, a sugar modified nucleotide is a nucleotide with a C3′ endo pucker conformation. In some embodiments, puckering refers to a non-planar conformation of a sugar ring, such as in a nucleotide. In puckering, an atom can be displaced from the plane of the sugar ring. In some embodiments, the pucker conformation is defined by the positions of C2′ and/or C3′ atoms relative to a plane formed by the C1′, O4′, and C4′ atoms of the sugar ring. In an “endo” pucker, displacement is on the beta-face of the ring, the same side as the C4′-C5′ bond and the base. In an exo pucker, the displacement is on the alpha-face of the ring, the opposite side of the plane as the C4′-C5′ bond. Thus, in some embodiments, in a nucleotide with a C3′ endo pucker conformation, the C3′ carbon is displaced to the same side as the C4′-C5′ bond, relative to the plane formed by the C1′, O4′, and C4′ atoms of the sugar ring. FIG. 1C, for example, shows 2′-OMeRNA, an exemplary nucleotide with a 3′ endo pucker conformation.

In some embodiments, a nucleotide analog is a locked nucleic acid (LNA) nucleotide. In some embodiments, an LNA nucleotide is also known as a bridged nucleic acid (BNA) nucleotide. In some embodiments, an LNA nucleotide is a nucleotide comprising a methylene bridge between the 2′O and 4′C of the sugar ring. In some embodiments, the methylene bridge conformationally locks the sugar into an N-type pucker conformation.

In some embodiments, a modified nucleotide, and/or a probe comprising a modified nucleotide, such as any circular probe, circularizable probe or probe set, or circularized probe described herein, comprises a modified linkage. In some embodiments, a modified linkage is any linkage that differs from an unmodified (e.g., naturally occurring) phosphodiester linkage, for example as observed in DNA and RNA. In some embodiments, a modified nucleotide is a nucleotide that is connected to an adjacent nucleotide by a modified linkage, but that is otherwise not modified.

In some embodiments, the linkage is a triazole linkage, or triazole-based linkage. In some embodiments, a triazole is a five-membered ring containing three nitrogen atoms and two carbon atoms. In some embodiments, a linkage between two residues comprises a triazole. FIG. 1D shows an exemplary modified nucleotide or nucleotide analog residue comprising a triazole linkage. In some aspects, the positioning of the nitrogen atoms within different triazole rings can differ. In some embodiments, the linkage is a thiol linkage, or thiol-based linkage. In some embodiments, the linkage is a thiophosphate or phosphorothioate linkage. In some embodiments, a thiophosphate, phosphorothioate linkage, or phosphorothioate internucleotide linkage, is a linkage wherein one of the phosphate oxygens (e.g., a non-linking oxygen) is substituted by a sulfur atom. In some embodiments, one or more modified nucleotides, such as nucleotide residues, comprise phosphorothioate backbone nucleotides, thiophosphate backbone nucleotides, and/or triazole-modified nucleotides. In some embodiments, a phosphorothioate backbone nucleotide is a nucleotide residue comprising a phosphorothioate linkage. In some embodiments, a thiophosphate backbone nucleotide is a nucleotide residue comprising a thiophosphate linkage. In some embodiments, a triazole-modified nucleotide is a nucleotide residue comprising a triazole-based linkage.

In some embodiments, a nucleotide or analog thereof is any nucleotide described herein, such as an unmodified nucleotide, or a modified nucleotide. In some embodiments, a nucleotide analog is a modified nucleotide. In some embodiments, an incorporable nucleotide or analog thereof is a nucleotide or analog thereof (such as a modified nucleotide) that is capable of being incorporated into a nucleic acid by a polymerase, for example at the 3′ end of an RCA product by phi29 DNA polymerase using a circularized probe as template. In some embodiments, a non-incorporable nucleotide or analog thereof is a nucleotide or analog thereof (such as a modified nucleotide) that is not capable of being incorporated into a nucleic acid by a polymerase, for example at the 3′ end of an RCA product by phi29 DNA polymerase using a circularized probe as template. In some embodiments, a non-incorporable nucleotide or analog thereof comprises a monophosphate nucleotide. In some embodiments, a monophosphate nucleotide is a nucleotide monomer (e.g., unincorporated nucleotide) having a single phosphate, for example as shown in FIG. 2B (left), which shows the structure of a deoxyribonucleoside monophosphate. In some embodiments, an incorporable nucleotide or analog thereof comprises a diphosphate nucleotide. In some embodiments, a diphosphate nucleotide is a nucleotide monomer (e.g., unincorporated nucleotide) having two phosphates, for example as shown in FIG. 2B (middle), which shows the structure of a deoxyribonucleotide diphosphate. Incorporable and non-incorporable nucleotides or analogs thereof are further described, for example, in Section IV.A.

In some embodiments, a nucleotide provided herein, such as a modified nucleotide or an incorporable nucleotide or analog thereof, is an alpha-thiol nucleotide. In some aspects, phosphoryl groups of a nucleoside triphosphate, starting from the group closest to the ribose, are referred to as the alpha phosphate, beta phosphate, and gamma phosphate, respectively. In some embodiments, an alpha-thiol nucleotide is a nucleotide in which one of the oxygens bonded to the alpha phosphate is substituted with a sulfur atom. An exemplary structure of an alpha thiol nucleotide is shown in FIG. 2B (right). The “base” in the figure can be any unmodified or modified nucleobase, such as any described herein. In some embodiments, an alpha-thiol nucleotide can be alpha-thio-dATP, alpha-thio-dTTP, alpha-thio-dCTP, or alpha-thio-dGTP.

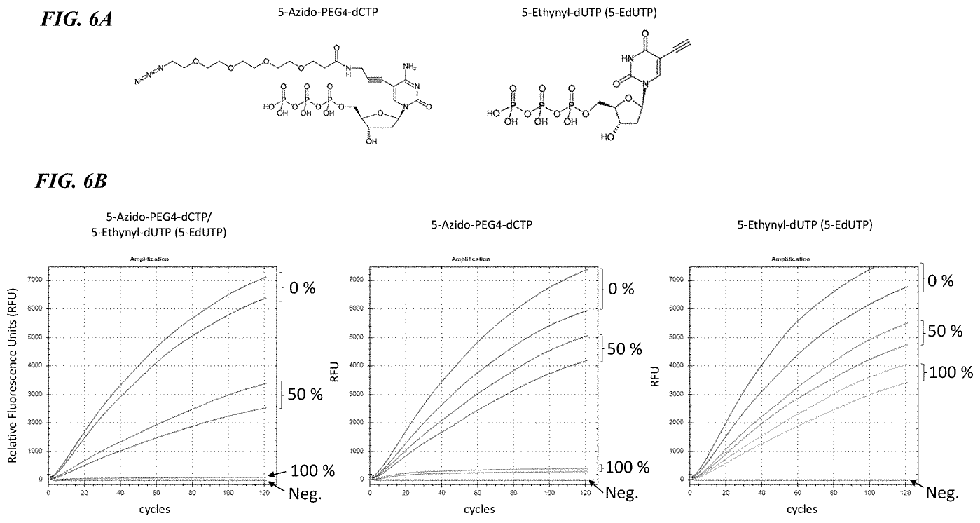

In some embodiments, a nucleotide provided herein, such as a modified nucleotide, or an incorporable nucleotide or analog thereof, is an alkyne modified nucleotide. In some aspects, an alkyne is an unsaturated hydrocarbon containing at least one carbon-carbon triple bond. In some embodiments, an alkyne modified nucleotide comprises an alkyne. In some embodiments, unmodified nucleobases (and their corresponding nucleotides) do not comprise alkynes. In some embodiments, an alkyne modified nucleotide comprises an alkyne modification on the nucleobase. Examples of alkyne modified nucleotides include 5-Azido-PEG₄-dCTP and 5-Ethynyl-dUTP (5-EdUTP) (e.g., as shown in FIG. 6A).

In some embodiments, a nucleotide provided herein, such as a modified nucleotide or an incorporable nucleotide or analog thereof, is an azide modified nucleotide. In some aspects, an azide modified nucleotide is a nucleotide that comprises an azide. In some aspects, and azide modified nucleotide is a nucleotide that comprises an azide modification. In some aspects, the azide modification is on any suitable part of the nucleotide. For example, in some embodiments, 8-azido-adenine is an azide modified adenine nucleotide with an azide modification in the nucleobase. In another example, 2′-azido nucleotides comprise an azide modification in the sugar moiety. In another example, azide modifications can be made to the phosphates (e.g. an azide can replace the phosphates) of a nucleoside triphosphate. In some embodiments, an azide modified nucleotide comprises 5-Azido-PEG₄-dCTP (e.g., as shown in FIG. 6A). It can be seen that a modified nucleotide can comprise more than one modification. For example, 5-Azido-PEG₄-dCTP comprises both an azide modification and a alkyne modification.

In some embodiments, a nucleotide or analog thereof (such as a modified nucleotide) comprises a hydrophobic modification. In some embodiments, the hydrophobic modification is on any suitable part of the nucleotide, such as the base, the sugar moiety, and/or the one or more phosphate groups. In some embodiments, the hydrophobic modification is a base modification and can be at any one or more positions on the base of the nucleotide.

In some embodiments, a hydrophobic modification is a modification that increases the hydrophobicity of a nucleotide. In some embodiments, the increased hydrophobicity results from the addition of nonpolar groups or molecules, such as carbon chains and/or hydrocarbon rings. In some embodiments, a hydrophobic modification can comprise a carbon chain and/or hydrocarbon ring. In some embodiments, the hydrophobic modification comprises a double bond and/or a triple bond, such as a double bond or triple bond between two carbon atoms. In some embodiments, the hydrophobic modification comprises a vinyl or ethynyl group. In some embodiments, a vinyl group is a functional group with the formula —CH═CH₂. In some embodiments, the vinyl group is attached to the nucleobase of the nucleotide or analog thereof, such as any of adenine, cytosine, guanine, thymine, and uracil. In some embodiments, the nucleotide or analog thereof is a vinyl-dNTP, such as vinyl-dUTP. In some embodiments, a vinyl-dUTP is a dUTP with a vinyl modification, e.g., a dUTP comprising a vinyl group. In some embodiments, the nucleotide or analog thereof is 5-vinyl-deoxyuridine triphosphate, e.g. as shown in FIG. 7. In some embodiments, the hydrophobic modification comprises an ethynyl group. In some embodiments, an ethynyl group is a functional group with the formula —C═CH. In some embodiments, the ethynyl group is attached to the nucleobase of the nucleotide or analog thereof, including any of adenine, cytosine, guanine, thymine, and uracil. In some embodiments, the nucleotide or analog thereof is an ethynyl-dNTP, such as ethynyl-dUTP. In some embodiments, the nucleotide or analog thereof is 5-ethynyl-dUTP (5-EdUTP), e.g. as shown in FIG. 7.

In some embodiments, incorporation of one or more nucleotide analogs comprising a hydrophobic modification in an RCA product increases overall hydrophobicity of the RCA product, for example, as compared to a reference RCA product not comprising the nucleotide analogs. In some embodiments, incorporation of one or more nucleotide analogs comprising a hydrophobic modification in an RCA product promotes base stacking interactions among nucleotides in the RCA product. In some aspects, base stacking refers to an arrangement of nucleobases found in the three dimensional structure of nucleic acids, in which two or more planar bases are arranged in close proximity in a stacked configuration. In some aspects, base stacking leads to exclusion of water, and increased base hydrophobicity (e.g. via a hydrophobic modification) can further promote base stacking. In some aspects, base stacking contributes to the shape and/or size of RCA products. In some embodiments, promoting base stacking among nucleotides in the RCA product (e.g. by inclusion of nucleotide analogs comprising hydrophobic modifications) can lead to decreased RCA product size, e.g. diameter.

### VI. Signal Amplification, Detection and Analysis

In some aspects, the provided methods involve analyzing, e.g., detecting or determining, one or more sequences present in the circular RCA template (e.g., a circular probe or circularized probe) and/or in an RCA product thereof. In some cases, the analysis is performed on one or more images captured, and may comprise processing the image(s) and/or quantifying signals observed. For example, the analysis may comprise processing information of one or more cell types, one or more types of biomarkers, a number or level of a biomarker, and/or a number or level of cells detected in a particular region of the sample. In some embodiments, the analysis comprises detecting a sequence e.g., a barcode present in the sample. In some embodiments, the analysis includes quantification of puncta (e.g., if amplification products are detected). In some cases, the analysis includes determining whether particular cells and/or signals are present that correlate with one or more biomarkers from a particular panel. In some embodiments, the obtained information may be compared to a positive and negative control, or to a threshold of a feature to determine if the sample exhibits a certain feature or phenotype. In some cases, the information may comprise signals from a cell, a region, and/or comprise readouts from multiple detectable labels. In some case, the analysis further includes displaying the information from the analysis or detection step. In some embodiments, software may be used to automate the processing, analysis, and/or display of data.

In some embodiments, detecting the RCA product in the biological sample comprises contacting the sample with nucleic acid probes that hybridize to the RCA product. After contacting the nucleic acid probes with a sample, the probes may be directly detected by determining detectable labels (if present), and/or detected by using one or more other probes that bind directly or indirectly to the probes or products thereof. The one or more other probes may comprise a detectable label. For instance, a first nucleic acid probe can bind to an RCA product in the sample, and a second nucleic acid probe can be introduced to bind to the first nucleic acid probe, where the second nucleic acid probe or a product thereof can then be detected using detectably labeled probes. Higher order probes that directly or indirectly bind to the second nucleic acid probe or product thereof may also be used, and the higher order probes or products thereof can then be detected using detectably labeled probes.

In some embodiments, the detection may be spatial, e.g., in two or three dimensions. In some embodiments, the detection may be quantitative, e.g., the amount or concentration an RCA product (and of a corresponding target nucleic acid) may be determined. In some embodiments, the primary probes, secondary probes, higher order probes, and/or detectably labeled probes may comprise any of a variety of entities able to hybridize a nucleic acid, e.g., DNA, RNA, LNA, and/or PNA, etc., depending on the application.

Detection probes may comprise recognition sequences that hybridize to sequences in the RCA product (e.g., to barcode sequences or complements thereof comprised by the RCA product). The recognition sequences may be of any length, and multiple recognition sequences in the same or different secondary nucleic acid probes may be of the same or different lengths. If more than one recognition sequence is used, the recognition sequences may independently have the same or different lengths. For instance, the recognition sequence may be at least 4, at least 5, least 6, least 7, least 8, least 9, at least 10, least 11, least 12, least 13, least 14, at least 15, least 16, least 17, least 18, least 19, at least 20, at least 25, at least 30, at least 35, at least 40, or at least 50 nucleotides in length. In some embodiments, the recognition sequence may be no more than 48, no more than 40, no more than 32, no more than 24, no more than 16, no more than 12, no more than 10, no more than 8, or no more than 6 nucleotides in length. Combinations of any of these are also possible, e.g., the recognition sequence may have a length of between 5 and 8, between 6 and 12, or between 7 and 15 nucleotides, etc. In one embodiment, the recognition sequence is of the same length as a barcode sequence or complement thereof of a circular or circularized probe or a product thereof. In some embodiments, the recognition sequence may be at least 50%, at least 60%, at least 70%, at least 75%, at least 80%, at least 85%, at least 90%, at least 92%, at least 94%, at least 95%, at least 96%, at least 97%, at least 98%, at least 99%, or at least 100% complementary to the barcode sequence or complement thereof.

In some embodiments, the RCA product generated according to the methods described herein can be detected with a method that comprises signal amplification. Exemplary signal amplification methods include targeted deposition of detectable reactive molecules around the site of probe hybridization, targeted assembly of branched structures (e.g., bDNA or branched assay using locked nucleic acid (LNA)), programmed in situ growth of concatemers by enzymatic rolling circle amplification (RCA) (e.g., as described in US 2019/0055594, the content of which is herein incorporated by reference in its entirety), hybridization chain reaction, assembly of topologically catenated DNA structures using serial rounds of chemical ligation (clampFISH), signal amplification via hairpin-mediated concatemerization (e.g., as described in US 2020/0362398, the content of which is herein incorporated by reference in its entirety), e.g., primer exchange reactions such as signal amplification by exchange reaction (SABER) or SABER with DNA-Exchange (Exchange-SABER). In some embodiments, a non-enzymatic signal amplification method may be used.

In some embodiments, hybridization chain reaction (HCR) can be used for signal amplification. HCR is an enzyme-free nucleic acid amplification based on a triggered chain of hybridization of nucleic acid molecules starting from HCR monomers, which hybridize to one another to form a nicked nucleic acid polymer. This polymer is the product of the HCR reaction which is ultimately detected in order to indicate the presence of the target analyte. HCR is described in detail in Dirks and Pierce, 2004, PNAS, 101(43), 15275-15278 and in U.S. Pat. Nos. 7,632,641 and 7,721,721, the entire contents of each of which are incorporated herein by reference. See also US 2006/00234261; Chemeris et al, 2008 Doklady Biochemistry and Biophysics, 419, 53-55; Niu et al, 2010, 46, 3089-3091; Choi et al, 2010, Nat. Biotechnol. 28(11), 1208-1212; and Song et al, 2012, Analyst, 137, 1396-1401, the entire contents of each of which are incorporated herein by reference. HCR monomers typically comprise a hairpin, or other metastable nucleic acid structure. In the simplest form of HCR, two different types of stable hairpin monomer, referred to here as first and second HCR monomers, undergo a chain reaction of hybridization events to form a long nicked double-stranded DNA molecule when an “initiator” nucleic acid molecule is introduced. The HCR monomers have a hairpin structure comprising a double stranded stem region, a loop region connecting the two strands of the stem region, and a single stranded region at one end of the double stranded stem region. The single stranded region which is exposed (and which is thus available for hybridization to another molecule, e.g. initiator or other HCR monomer) when the monomers are in the hairpin structure may be known as the “toehold region” (or “input domain”). The first HCR monomers each further comprise a sequence which is complementary to a sequence in the exposed toehold region of the second HCR monomers. This sequence of complementarity in the first HCR monomers may be known as the “interacting region” (or “output domain”). Similarly, the second HCR monomers each comprise an interacting region (output domain), e.g. a sequence which is complementary to the exposed toehold region (input domain) of the first HCR monomers. In the absence of the HCR initiator, these interacting regions are protected by the secondary structure (e.g. they are not exposed), and thus the hairpin monomers are stable or kinetically trapped (also referred to as “metastable”), and remain as monomers (e.g. preventing the system from rapidly equilibrating), because the first and second sets of HCR monomers cannot hybridize to each other. However, once the initiator is introduced, it is able to hybridize to the exposed toehold region of a first HCR monomer, and invade it, causing it to open up. This exposes the interacting region of the first HCR monomer (e.g. the sequence of complementarity to the toehold region of the second HCR monomers), allowing it to hybridize to and invade a second HCR monomer at the toehold region. This hybridization and invasion in turn opens up the second HCR monomer, exposing its interacting region (which is complementary to the toehold region of the first HCR monomers), and allowing it to hybridize to and invade another first HCR monomer. The reaction continues in this manner until all of the HCR monomers are exhausted (e.g. all of the HCR monomers are incorporated into a polymeric chain). Ultimately, this chain reaction leads to the formation of a nicked chain of alternating units of the first and second monomer species. The presence of the HCR initiator is thus required in order to trigger the HCR reaction by hybridization to and invasion of a first HCR monomer. The first and second HCR monomers are designed to hybridize to one another are thus may be defined as cognate to one another. They are also cognate to a given HCR initiator sequence. HCR monomers which interact with one another (hybridize) may be described as a set of HCR monomers or an HCR monomer, or hairpin, system.

An HCR reaction could be carried out with more than two species or types of HCR monomers. For example, a system involving three HCR monomers could be used. In such a system, each first HCR monomer may comprise an interacting region which binds to the toehold region of a second HCR monomer; each second HCR may comprise an interacting region which binds to the toehold region of a third HCR monomer; and each third HCR monomer may comprise an interacting region which binds to the toehold region of a first HCR monomer. The HCR polymerization reaction would then proceed as described above, except that the resulting product would be a polymer having a repeating unit of first, second and third monomers consecutively. Corresponding systems with larger numbers of sets of HCR monomers could readily be conceived.

In some embodiments, similar to HCR reactions that use hairpin monomers, linear oligo hybridization chain reaction (LO-HCR) can also be used for signal amplification. In some embodiments, provided herein is a method of detecting an analyte in a sample comprising: (i) performing a linear oligo hybridization chain reaction (LO-HCR), wherein an initiator is contacted with a plurality of LO-HCR monomers of at least a first and a second species to generate a polymeric LO-HCR product hybridized to a target nucleic acid molecule, wherein the first species comprises a first hybridization region complementary to the initiator and a second hybridization region complementary to the second species, wherein the first species and the second species are linear, single-stranded nucleic acid molecules; wherein the initiator is provided in one or more parts, and hybridizes directly or indirectly to or is comprised in the target nucleic acid molecule; and (ii) detecting the polymeric product, thereby detecting the analyte. In some embodiments, the first species and/or the second species may not comprise a hairpin structure. In some embodiments, the plurality of LO-HCR monomers may not comprise a metastable secondary structure. In some embodiments, the LO-HCR polymer may not comprise a branched structure. In some embodiments, performing the linear oligo hybridization chain reaction comprises contacting the target nucleic acid molecule with the initiator to provide the initiator hybridized to the target nucleic acid molecule. In any of the embodiments herein, the target nucleic acid molecule and/or the analyte can be an RCA product.

In some embodiments, detection of nucleic acids sequences in situ includes combination of an RCA method described herein with an assembly for branched signal amplification. In some embodiments, the assembly complex comprises an amplifier hybridized directly or indirectly (via one or more oligonucleotides) to a sequence of the RCA product. In some embodiments, the assembly includes one or more amplifiers each including an amplifier repeating sequence. In some aspects, the one or more amplifiers is labeled. Described herein is a method of using the aforementioned assembly, including for example, using the assembly in multiplexed error-robust fluorescent in situ hybridization (MERFISH) applications, with branched DNA amplification for signal readout. In some embodiments, the amplifier repeating sequence is about 5-30 nucleotides, and is repeated N times in the amplifier. In some embodiments, the amplifier repeating sequence is about 20 nucleotides, and is repeated at least two times in the amplifier. In some aspects, the one or more amplifier repeating sequence is labeled. For exemplary branched signal amplification, see e.g., U.S. Pat. Pub. Nos. US 2020/0399689 and US 2022/0064697, and Xia et al., Multiplexed Detection of RNA using MERFISH and branched DNA amplification, Scientific Reports (2019), the entire contents of each of which are incorporated herein by reference.

In some embodiments, the RCA product can be detected in with a method that comprises signal amplification by performing a primer exchange reaction (PER). In various embodiments, a primer with domain on its 3′ end binds to a catalytic hairpin, and is extended with a new domain by a strand displacing polymerase. For example, a primer with domain 1 on its 3′ ends binds to a catalytic hairpin, and is extended with a new domain 1 by a strand displacing polymerase, with repeated cycles generating a concatemer of repeated domain 1 sequences. In various embodiments, the strand displacing polymerase is Bst. In various embodiments, the catalytic hairpin includes a stopper which releases the strand displacing polymerase. In various embodiments, branch migration displaces the extended primer, which can then dissociate. In various embodiments, the primer undergoes repeated cycles to form a concatemer primer. In various embodiments, a plurality of concatemer primers is contacted with a sample comprising RCA products generated using methods described herein. In various embodiments, the RCA product may be contacted with a plurality of concatemer primers and a plurality of labeled probes. see e.g., U.S. Pat. Pub. No. US20190106733, which is incorporated herein by reference, for exemplary molecules and PER reaction components.

In some embodiments, the RCA product can be detected by providing detection probes, such as probes for performing a chain reaction that forms an amplification product, e.g., HCR. In some embodiments, the analysis comprises determining the sequence of all or a portion of the amplification product. In some embodiments, the analysis comprises detecting a sequence present in the amplification product. In some embodiments, the sequence of all or a portion of the amplification product is indicative of the identity of a region of interest in a target nucleic acid. In other embodiments, the provided methods involve analyzing, e.g., detecting or determining, one or more sequences present in the polynucleotide probes (e.g., a barcode sequence present in an overhang region of the first and/or second probe).

In some embodiments, the methods comprise sequencing all or a portion of the RCA product, such as one or more barcode sequences present in the RCA product. In some embodiments, the analysis and/or sequence determination comprises sequencing all or a portion of the RCA product or the probe(s) and/or in situ hybridization to the RCA product or the probe(s). In some embodiments, the sequencing step involves sequencing by hybridization, sequencing by ligation, and/or fluorescent in situ sequencing, hybridization-based in situ sequencing and/or wherein the in situ hybridization comprises sequential fluorescent in situ hybridization. In some embodiments, the analysis and/or sequence determination comprises detecting a polymer generated by a hybridization chain reaction (HCR) reaction, see e.g., US 2017/0009278, which is incorporated herein by reference, for exemplary probes and HCR reaction components. In some embodiments, the detection or determination comprises hybridizing to the amplification product a detection oligonucleotide labeled with a fluorophore, an isotope, a mass tag, or a combination thereof. In some embodiments, the detection or determination comprises imaging the amplification product. In some embodiments, the target nucleic acid is an mRNA in a tissue sample, and the detection or determination is performed when the target nucleic acid and/or the amplification product is in situ in the tissue sample.

In some aspects, the provided methods comprise imaging the amplification product (e.g., amplicon) and/or one or more portions of the polynucleotides, for example, via binding of the detection probe and detecting the detectable label. In some embodiments, the detection probe comprises a detectable label that can be measured and quantitated. Label and detectable label comprise a directly or indirectly detectable moiety that is associated with (e.g., conjugated to) a molecule to be detected, e.g., a detectable probe, comprising, but not limited to, fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

A fluorophore comprises a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. Autofluorescence is a term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

In some embodiments, a detectable probe containing a detectable label can be used to detect one or more polynucleotide(s) and/or amplification products (e.g., amplicon) described herein. In some embodiments, the methods involve incubating the detectable probe containing the detectable label with the sample, washing unbound detectable probe, and detecting the label, e.g., by imaging.

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991), the entire contents of each of which are incorporated herein by reference. In some embodiments, exemplary techniques, methods and methodologies applicable to the provided embodiments comprise those described in, for example, U.S. Pat. Nos. 4,757,141, 5,151,507 and 5,091,519, the entire contents of each of which are incorporated herein by reference. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes), the entire contents of each of which are incorporated herein by reference. Labelling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264, the entire contents of each of which are incorporated herein by reference. In some embodiments, a fluorescent label is a signaling moiety that conveys information through the fluorescent absorption and/or emission properties of one or more molecules. Exemplary fluorescent properties comprise fluorescence intensity, fluorescence lifetime, emission spectrum characteristics and energy transfer.

Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Methods for custom synthesis of nucleotides having other fluorophores are described, for example, in Henegariu et al. (2000) Nature Biotechnol. 18:345, the content of which is herein incorporated by reference in its entirety.

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences. See, for example, Lakowicz et al. (2003) Bio Techniques 34:62, the content of which is herein incorporated by reference in its entirety.

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection. In some embodiments, an antibody is an antibody molecule of any class, or any sub-fragment thereof, such as a Fab.

Other suitable labels for a polynucleotide sequence may comprise fluorescein (FAM), digoxigenin, dinitrophenol (DNP), dansyl, biotin, bromodeoxyuridine (BrdU), hexahistidine (6xHis), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or an polynucleotide sequence can be indirectly labeled, especially with a hapten that is then bound by a capture agent, e.g., as disclosed in U.S. Pat. Nos. 5,344,757, 5,702,888, 5,354,657, 5,198,537, 4,849,336, and 5,073,562, the entire contents of each of which are incorporated herein by reference. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the analysis and/or sequence determination can be carried out at room temperature for best preservation of tissue morphology with low background noise and error reduction. In some embodiments, the analysis and/or sequence determination comprises eliminating error accumulation as sequencing proceeds.

In some embodiments, the analysis and/or sequence determination involves washing to remove unbound polynucleotides, thereafter revealing a fluorescent product for imaging.

In some aspects, the detecting involves using detection methods such as flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the flow cytometry is mass cytometry or fluorescence-activated flow cytometry. In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any of a number of different types of microscopy, e.g., confocal microscopy, two-photon microscopy, light-field microscopy, intact tissue expansion microscopy, and/or CLARITY™ optimized light sheet microscopy (COLM).

In some embodiments, fluorescence microscopy is used for detection and imaging of the detection probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detection probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, a nucleic acid probe, such as a circular probe or circularizable probe or probe set, or a nucleic acid probe that hybridizes to an RCA product, may comprise 1, 2, 3, 4, 5, 6, 7, 8, 9, 10, 11, 12, 13, 14, 15 or more, 20 or more, 32 or more, 40 or more, or 50 or more barcode sequences. The barcode sequences may be positioned anywhere within the nucleic acid probe. If more than one barcode sequences are present, the barcode sequences may be positioned next to each other, and/or interspersed with other sequences. In some embodiments, two or more of the barcode sequences may also at least partially overlap. In some embodiments, two or more of the barcode sequences in the same probe do not overlap. In some embodiments, all of the barcode sequences in the same probe are separated from one another by at least a phosphodiester bond (e.g., they may be immediately adjacent to each other but do not overlap), such as 1, 2, 3, 4, 5, 6, 7, 8, 9, 10 or more nucleotides apart.

The barcode sequences, if present, may be of any length. If more than one barcode sequence is used, the barcode sequences may independently have the same or different lengths, such as at least 5, at least 10, at least 15, at least 20, at least 25, at least 30, at least 35, at least 40, at least 50 nucleotides in length. In some embodiments, the barcode sequence may be no more than 120, no more than 112, no more than 104, no more than 96, no more than 88, no more than 80, no more than 72, no more than 64, no more than 56, no more than 48, no more than 40, no more than 32, no more than 24, no more than 16, or no more than 8 nucleotides in length. Combinations of any of these are also possible, e.g., the barcode sequence may be between 5 and 10 nucleotides, between 8 and 15 nucleotides, etc.

The barcode sequence may be arbitrary or random. In certain cases, the barcode sequences are chosen so as to reduce or minimize homology with other components in a sample, e.g., such that the barcode sequences do not themselves bind to or hybridize with other nucleic acids suspected of being within the cell or other sample. In some embodiments, between a particular barcode sequence and another sequence (e.g., a cellular nucleic acid sequence in a sample or other barcode sequences in probes added to the sample), the homology may be less than 10%, less than 8%, less than 7%, less than 6%, less than 5%, less than 4%, less than 3%, less than 2%, or less than 1%. In some embodiments, the homology may be less than 20, 19, 18, 17, 16, 15, 14, 13, 12, 11, 10, 9, 8, 7, 6, 5, 4, 3, or 2 bases, and in some embodiments, the bases are consecutive bases.

In some embodiments, the number of distinct barcode sequences in a population of circular probes or circularizable probes or probe sets is less than the number of distinct targets (e.g., nucleic acid analytes and/or protein analytes) of the nucleic acid probes, and yet the distinct targets may still be uniquely identified from one another, e.g., by encoding a probe with a different combination of barcode sequences. However, not all possible combinations of a given set of barcode sequences need be used. For instance, each probe may contain 1, 2, 3, 4, 5, 6, 7, 8, 9, 10, 11, 12, 13, 14, 15, etc. or more barcode sequences. In some embodiments, a population of nucleic acid probes may each contain the same number of barcode sequences, although in other cases, there may be different numbers of barcode sequences present on the various probes.

In some embodiments, a method disclosed herein comprises multiplexed analysis of a biological sample comprising consecutive cycles of probe hybridization to an RCA product, fluorescence imaging, and signal removal, where the signal removal comprises removing the fluorophore from a fluorophore-labeled reactive molecule (e.g., tyramide). Exemplary detectable reactive reagents and methods are described in U.S. Pat. No. 6,828,109, US 2019/0376956, US 2019/0376956, US 2022/0026433, US 2022/0128565, and US 2021/0222234, each of which are incorporated herein by reference in their entireties.

In some embodiments, detecting the RCA product in the sample may comprise sequencing all or a portion of the RCA product (e.g., one or more barcode sequences or complements thereof comprised by the RCA product). In some embodiments, sequencing can be performed in situ. In situ sequencing typically involves incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the identities (e.g., nucleotide sequence) of the incorporated nucleotides or labeled primer extension products can be determined, and consequently, the nucleotide sequence of the corresponding template nucleic acid. Aspects of in situ sequencing are described, for example, in Mitra et al., (2003) Anal. Biochem. 320, 55-65, and Lee et al., (2014) Science, 343(6177), 1360-1363, the entire contents of each of which are incorporated herein by reference. In addition, examples of methods and systems for performing in situ sequencing are described in US 2016/0024555, US 2019/0194709, and in U.S. Pat. Nos. 10,138,509, 10,494,662 and 10,179,932, the entire contents of each of which are incorporated herein by reference. Exemplary techniques for in situ sequencing comprise, but are not limited to, STARmap (described for example in Wang et al., (2018) Science, 361(6499) 5691, the content of which is herein incorporated by reference in its entirety), MERFISH (described for example in Moffitt, (2016) Methods in Enzymology, 572, 1-49, the content of which is herein incorporated by reference in its entirety), hybridization-based in situ sequencing (HybISS) (described for example in Gyllborg et al., Nucleic Acids Res (2020) 48(19):el 12, the content of which is herein incorporated by reference in its entirety) and FISSEQ (described for example in US 2019/0032121, the content of which is herein incorporated by reference in its entirety).

In some embodiments, sequencing can be performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Exemplary SBS methods comprise those described for example, but not limited to, US 2007/0166705, US 2006/0188901, U.S. Pat. No. 7,057,026, US 2006/0240439, US 2006/0281109, US 2011/005986, US 2005/0100900, U.S. Pat. No. 9,217,178, US 2009/0118128, US 2012/0270305, US 2013/0260372, and US 2013/0079232, the entire contents of each of which are incorporated herein by reference.

In some embodiments, sequencing can be performed by sequential fluorescence hybridization (e.g., sequencing by hybridization). Sequential fluorescence hybridization can involve sequential hybridization of detection probes comprising an oligonucleotide and a detectable label.

In some embodiments, sequencing can be performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309: 1728-1732, and in U.S. Pat. Nos. 5,599,675; 5,750,341; 6,969,488; 6,172,218 and 6,306,597, the entire contents of each of which are incorporated herein by reference.

In some embodiments, the barcodes or complements thereof comprised by the RCA product or by one or more probes hybridized thereto are targeted by detectably labeled detection oligonucleotides, such as fluorescently labeled oligonucleotides. In some embodiments, one or more decoding schemes are used to decode the signals, such as fluorescence, for sequence determination. In any of the embodiments herein, barcodes can be analyzed (e.g., detected or sequenced) using any suitable methods or techniques, comprising those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), hybridization-based in situ sequencing (HybISS), in situ sequencing, targeted in situ sequencing, fluorescent in situ sequencing (FISSEQ), or spatially-resolved transcript amplicon readout mapping (STARmap). In some embodiments, the methods provided herein comprise analyzing the barcodes by sequential hybridization and detection with a plurality of labelled probes (e.g., detection oligonucleotides). Exemplary decoding schemes are described in Eng et al., “Transcriptome-scale Super-Resolved Imaging in Tissues by RNA SeqFISH+,” Nature 568(7751):235-239 (2019); Chen et al., “Spatially resolved, highly multiplexed RNA profiling in single cells,” Science; 348(6233):aaa6090 (2015); Gyllborg et al., Nucleic Acids Res (2020) 48(19):el 12; U.S. Pat. No. 10,457,980 B2; US 2016/0369329 A1; WO 2018/026873 A1; and US 2017/0220733 A1, each of which are incorporated by reference in their entirety. In some embodiments, these assays enable signal amplification, combinatorial decoding, and error correction schemes at the same time.

In some embodiments, nucleic acid hybridization can be used for sequencing. These methods utilize labeled nucleic acid decoder probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004), the entire contents of each of which are incorporated herein by reference.

In some embodiments, real-time monitoring of DNA polymerase activity can be used during sequencing. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and Korlach et al., Proc. Natl. Acad. Sci. USA (2008), 105, 1176-1181, the entire contents of each of which are incorporated herein by reference.

In some embodiments, sequence analysis of nucleic acids (e.g., nucleic acids such as probes or RCPs comprising barcode sequences) can be performed by sequential hybridization (e.g., sequencing by hybridization and/or sequential in situ fluorescence hybridization). Sequential fluorescence hybridization can involve sequential hybridization of detectable probes comprising an oligonucleotide and a detectable label. In some embodiments, a method disclosed herein comprises sequential hybridization of the detectable probes disclosed herein, including detectably labeled probes (e.g., fluorophore conjugated oligonucleotides) and/or probes that are not detectably labeled per se but are capable of binding (e.g., via nucleic acid hybridization) and being detected by detectably labeled probes. Exemplary methods comprising sequential fluorescence hybridization of detectable probes are described in US 2019/0161796, US 2020/0224244, US 2022/0010358, US 2021/0340618, and WO 2021/138676, all of which are incorporated herein by reference. In some embodiments, the methods provided herein can include analyzing the identifier sequences (e.g., analyte sequences or barcode sequences) by sequential hybridization and detection with a plurality of labeled probes (e.g., detection oligonucleotides).

In some embodiments, sequence detection comprises contacting the biological sample with one or more intermediate probes that directly or indirectly hybridize to the RCP, wherein the one or more intermediate probes are detectable using one or more detectably-labeled probes (e.g., probes comprising branched linkers, such as the probes described in Section II.B), and dehybridizing the one or more intermediate probes and/or the one or more detectably-labeled probes from the rolling circle amplification product. In some aspects, dehybridizing probes comprises removal of the probes from the RCP. In some embodiments, the one or more intermediate probes comprise one or more overhang regions (e.g., a 5′ and/or 3′ end of the probe that does not hybridize to the rolling circle amplification product). A probe comprising a single overhang region may be referred to as an “L-shaped probe,” and a probe comprising two overhangs may be referred to as a “U-shaped probe.” In some cases, the overhang region comprises a binding region for binding one or more detectably-labeled probes. In some embodiments, the detecting comprises contacting the biological sample with a pool of intermediate probes corresponding to different barcode sequences or portions thereof, and a pool of detectably-labeled probes corresponding to different detectable labels. In some embodiments, the biological sample is sequentially contacted with different pools of intermediate probes. In some instances, a common or universal pool of detectably-labeled probes is used in a plurality of sequential hybridization steps (e.g., with different pools of intermediate probes).

In some embodiments, detection of the barcode sequences is performed by sequential hybridization of probes to the barcode sequences or complements thereof and detecting complexes formed by the probes and barcode sequences or complements thereof. In some cases, each barcode sequence or complement thereof is assigned a sequence of signal codes that identifies the barcode sequence or complement thereof (e.g., a temporal signal signature or code that identifies the analyte), and detecting the barcode sequences or complements thereof can comprise decoding the barcode sequences of complements thereof by detecting the corresponding sequences of signal codes detected from sequential hybridization, detection, and removal of sequential pools of intermediate probes and the universal pool of detectably labeled probes. In some cases, the sequences of signal codes can be fluorophore sequences assigned to the corresponding barcode sequences or complements thereof. In some embodiments, the detectably labeled probes are fluorescently labeled. In some embodiments, the barcode sequence or complement thereof is decoded by sequential probe hybridization as described in US 2021/0340618, the content of which is herein incorporated by reference in its entirety.

In any of the embodiments herein, the detecting step can comprise contacting the biological sample with one or more detectably labeled probes that directly or indirectly hybridize to the barcode sequences or complements thereof (e.g., in amplification products generated using the probes or probe sets), and dehybridizing the one or more detectably labeled probes. In any of the embodiments herein, the contacting and dehybridizing steps can be repeated with the one or more detectably labeled probes and/or one or more other detectably labeled probes that directly or indirectly hybridize to the barcode sequences or complements thereof. In some aspects, the method comprises sequential hybridization of detectably labeled probes to create a spatiotemporal signal signature or code that identifies the analyte.

In any of the embodiments herein, the detecting step can comprise contacting the biological sample with one or more first detectably labeled probes that directly hybridize to the plurality of probes or probe sets. In some instances, the detecting step can comprise contacting the biological sample with one or more first detectably labeled probes that indirectly hybridize to the plurality of probes or probe sets. In any of the embodiments herein, the detecting step can comprise contacting the biological sample with one or more second detectably labeled probes that directly or indirectly hybridize to the plurality of probes or probe sets.

In any of the embodiments herein, the detecting step can comprise contacting the biological sample with one or more intermediate probes that directly or indirectly hybridize to the barcode sequences or complements thereof (e.g., of the plurality of probes or probe sets or rolling circle amplification product generated using the plurality of probes or probe sets), wherein the one or more intermediate probes are detectable using one or more detectably labeled probes. In any of the embodiments herein, the detecting step can further comprise dehybridizing the one or more intermediate probes and/or the one or more detectably labeled probes from the barcode sequences or complements thereof (e.g., of the plurality of probes or probe sets or rolling circle amplification product generated using the plurality of probes or probe sets). In any of the embodiments herein, the contacting and dehybridizing steps can be repeated with the one or more intermediate probes, the one or more detectably labeled probes, one or more other intermediate probes, and/or one or more other detectably labeled probes.

In some aspects, provided herein is a method of detecting RCPs and decoding identifier sequences in the RCPs, comprising: a) providing intermediate probes and fluorescently labeled probes, and RCPs in a cell or tissue sample (e.g., a fixed sample, an FFPE sample, or a hydrogel-embedded and cleared sample), each RCP comprising multiple copies of an identifier sequence having an assigned signal code sequence from a codebook; b) in a first cycle, delivering (e.g., using a fluidics module of an instrument or system) to the cell or tissue sample a first plurality of intermediate probe/fluorescently labeled probe pairs, wherein the intermediate probe and the fluorescently labeled probe in each pair form a complex comprising the intermediate probe hybridized to an RCP of the plurality of RCPs and the fluorescently labeled probe hybridized to the intermediate probe, wherein the intermediate probe comprises (i) a recognition sequence complementary to the identifier sequence in the RCP and (ii) an overhang sequence, and wherein the fluorescently labeled probe comprises (i) a sequence complementary to the overhang sequence and (ii) a fluorescent label; c) detecting (e.g., using an optics module of the instrument or system) first signals (or absence thereof) associated with the fluorescent labels of the first plurality of probe pairs at multiple locations on the solid support, wherein the first signal or absence thereof detected at a particular location corresponds to a first signal code in the signal code sequence assigned to the identifier sequence in the RCP at the particular location; d) in a second cycle, delivering (e.g., using the fluidics module of the instrument or system) to the cell or tissue sample a second plurality of intermediate probe/fluorescently labeled probe pairs, wherein the intermediate probe and the fluorescently labeled probe in each pair form a complex comprising the intermediate probe hybridized to an RCP of the plurality of RCPs and the fluorescently labeled probe hybridized to the intermediate probe, wherein the intermediate probe comprises (i) a recognition sequence complementary to the identifier sequence in the RCP and (ii) an overhang sequence, and wherein the fluorescently labeled probe comprises (i) a sequence complementary to the overhang sequence and (ii) a fluorescent label; e) detecting (e.g., using the optics module of the instrument or system) second signals (or absence thereof) associated with the fluorescent labels of the second plurality of probe pairs at multiple locations on the solid support, wherein the second signal or absence thereof detected at a particular location corresponds to a second signal code in the signal code sequence assigned to the identifier sequence in the RCP at the particular location, thereby generating a signal code sequence comprising at least the first signal code and the second signal code at each of the multiple locations; f) comparing (e.g., using a system controller of the instrument or system) the generated signal code sequences for the RCPs at the multiple locations to those from the codebook, thereby decoding the identifier sequences in the RCPs.

In some embodiments, in the first cycle, a first pool of intermediate probes and a universal pool of fluorescently labeled probes are delivered to the solid support, wherein each intermediate probe in the first pool of intermediate probes comprises (i) a recognition sequence complementary to one of the different identifier sequences in the RCPs, and (ii) a hybridization sequence complementary to a fluorescently labeled probe of the universal pool. In some embodiments, in the second cycle, a second pool of intermediate probes and the universal pool of fluorescently labeled probes are delivered to the solid support, wherein each intermediate probe in the second pool of intermediate probes comprises (i) a recognition sequence complementary to one of the different identifier sequences in the RCPs, and (ii) a hybridization sequence complementary to a fluorescently labeled probe of the universal pool. In some embodiments, the number of different identifier sequences in the RCPs is at least 9 and the number of fluorescently labeled probes of different sequences in the universal pool is 4.

In any of the embodiments herein, each fluorescently labeled probe of a different sequence in the universal pool can be labeled with a fluorophore of a different color. In any of the embodiments herein, the signal code sequence comprising the first signal code, the second signal code, a third signal code corresponding to a third cycle, and a fourth signal code corresponding to a fourth cycle. In any of the embodiments herein, the signal code sequence can comprises a dark signal code corresponding to the absence of signal in the corresponding cycle.

### VII. Kits

Also provided herein are kits, for example comprising one or more polynucleotides, e.g., any of the circular probes or circularizable probes or probe sets described in Section III, and reagents for performing the methods provided herein, for example reagents required for one or more steps comprising hybridization, ligation, amplification, detection, sequencing, and/or sample preparation as described herein. In some embodiments, the kit further comprises a target nucleic acid, e.g., any described in Section II. In some embodiments, any or all of the polynucleotides are DNA molecules. In some embodiments, the target nucleic acid is a messenger RNA molecule. In some embodiments, the kit further comprises a ligase, for instance for forming a circularized probe from the circularizable probe or probe set. In some embodiments, the ligase has DNA-splinted DNA ligase activity. In some embodiments, the ligase has RNA-splinted ligase activity. In some embodiments, the kit further comprises a polymerase, for instance for performing rolling circle amplification of the circular or circularized probe, e.g., using any of the methods described in Section IV. In some embodiments, the kit further comprises a primer for amplification (e.g., an RCA primer).

In some embodiments, disclosed herein is a kit for analyzing a biological sample using rolling circle amplification, comprising: (a) a circular probe or circularizable probe or probe set comprising one or more modified nucleotide or nucleotide analog residues, wherein the circular probe or circularizable probe or probe set comprises a target hybridization region complementary to a target sequence of a target nucleic acid; and (b) a polymerase; wherein the one or more modified nucleotide or nucleotide analog residues decreases the polymerization rate of the polymerase in a rolling circle amplification reaction using the circular probe or a circularized probe generated from the circularizable probe or probe set as template as compared to a reference circular template without the one or more modified nucleotide or nucleotide analog residues. In some embodiments, the kit can further comprise a ligase for generating the circularized probe from the circularizable probe or probe set. In some embodiments, the one or more modified nucleotide or nucleotide analog residues can comprise modified sugar nucleotide residues and/or backbone modified nucleotide residues. In some embodiments, the one or more modified nucleotide or nucleotide analog residues can be selected from the group consisting of 2′-O-methyl ribonucleic acid (2′-OMeRNA), locked nucleic acid (LNA) nucleotides, 2′-fluoro ribonucleic acid (2′-F RNA), phosphorothioate backbone nucleotides, thiophosphate backbone nucleotides, triazole-modified nucleotides, and combinations thereof. In any of the preceding embodiments, the kit can further comprise one or more non-incorporable nucleotides or analogs thereof configured to bind transiently to the polymerase but not be incorporated by the polymerase, and/or one or more incorporable nucleotides or analogs thereof configured to be incorporated by the polymerase at a slower rate than a corresponding nucleoside triphosphate.

In some aspects, provided herein is a kit for performing rolling circle amplification (RCA), comprising: (a) a polymerase; (b) one or more non-incorporable nucleotides or analogs thereof configured to bind transiently to the polymerase but not be incorporated by the polymerase, and/or one or more incorporable nucleotides or analogs thereof configured to be incorporated by the polymerase at a slower rate than a corresponding nucleoside triphosphate; and (c) one or more circular probes to be used as a template for RCA, one or more circularizable probes or probe sets for generating a circularized probe to be used as a template for RCA, and/or one or more reagents for generating a circularized template for RCA. In some embodiments, the one or more non-incorporable nucleotides or analogs thereof and/or one or more incorporable nucleotides or analogs thereof can comprise an alpha-thiol nucleotide, an alkyne modified nucleotide, an azide modified nucleotide, a diphosphate nucleotide, and/or a monophosphate nucleotide. In any of the preceding embodiments, the kit can further comprise unmodified deoxyribose nucleotide triphosphates.

In any of the preceding embodiments of the kit, the combination of the one or more non-incorporable nucleotides or analogs thereof, the one or more incorporable nucleotides or analogs thereof, and/or the unmodified deoxyribose nucleotide triphosphates can be comprised by a reaction mixture for RCA, such as any of the reaction mixtures described in Section IV.

In some aspects, provided herein is a kit for performing rolling circle amplification (RCA); the kit comprising: (a) a polymerase; (b) one or more nucleotide analogs comprising one or more hydrophobic modifications configured to be incorporated by the polymerase; and (c) one or more circular probes or circularizable probes or probe sets for generating a circularized probe as a template for RCA, or one or more reagents for generating a circularized template for RCA. In some embodiments, the one or more nucleotide analogs comprise a modified dUTP selected from ethynyl-dUTP and/or vinyl dUTP. In some embodiments, the one or more nucleotide analogs are provided as a reaction mixture comprising the one or more nucleotide analogs and unmodified deoxyribose nucleotide triphosphates. In some embodiments, the ratio of the modified dUTP to an unmodified dTTP in the reaction mixture is at least about 80:20.

The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein, e.g., wash buffer and ligation buffer. In some embodiments, the kits contain reagents for detection and/or sequencing, such as barcode detection probes or detectable labels. In some embodiments, the kits optionally contain other components, for example nucleic acid primers, enzymes and reagents, buffers, nucleotides, modified nucleotides, reagents for additional assays.

### VIII. Applications

In some aspects, the provided embodiments can be applied in an in situ method of analyzing nucleic acid sequences, such as an in situ transcriptomic analysis or in situ sequencing, for example from intact tissues or samples in which the spatial information has been preserved. In some aspects, the embodiments can be applied in an imaging or detection method for multiplexed nucleic acid analysis. In some aspects, the provided embodiments can be used to identify or detect regions of interest in target nucleic acids.

In some embodiments, the region of interest comprises a single-nucleotide polymorphism (SNP). In some embodiments, the region of interest comprises is a single-nucleotide variant (SNV). In some embodiments, the region of interest comprises a single-nucleotide substitution. In some embodiments, the region of interest comprises a point mutation. In some embodiments, the region of interest comprises a single-nucleotide insertion.

In some aspects, the embodiments can be applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, spatially resolved gene expression analysis for biological investigation or drug screening. In clinical diagnostics, applications comprise, but are not limited to, detecting gene markers such as disease, immune responses, bacterial or viral DNA/RNA for patient samples.

In some aspects, the embodiments can be applied to visualize the distribution of genetically encoded markers in whole tissue at subcellular resolution, for example, chromosomal abnormalities (inversions, duplications, translocations, etc.), loss of genetic heterozygosity, the presence of gene alleles indicative of a predisposition towards disease or good health, likelihood of responsiveness to therapy, or in personalized medicine or ancestry.

### IX. Terminology

Specific terminology is used throughout this disclosure to explain various aspects of the apparatus, systems, methods, and compositions that are described.

Having described some illustrative embodiments of the present disclosure, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the present disclosure. In particular, although many of the examples presented herein involve specific combinations of method acts or system elements, it should be understood that those acts and those elements may be combined in other ways to accomplish the same objectives.

As used herein, the singular forms “a,” “an,” and “the” include plural referents unless the context clearly dictates otherwise. For example, “a” or “an” means “at least one” or “one or more.”

The term “about” as used herein refers to the usual error range for the respective value readily known to the skilled person in this technical field. Reference to “about” a value or parameter herein includes (and describes) embodiments that are directed to that value or parameter per se.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### (i) Barcode

A “barcode” is a label, or identifier, that conveys or is capable of conveying information (e.g., information about an analyte in a sample and/or a bead). A barcode can be part of an analyte, or independent of an analyte. A barcode can be attached to an analyte. A particular barcode can be unique relative to other barcodes.

Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”).

Barcodes can spatially-resolve molecular components found in biological samples, for example, at single-cell resolution (e.g., a barcode can be or can include a “spatial barcode”). In some embodiments, a barcode includes both a UMI and a spatial barcode. In some embodiments, a barcode includes two or more sub-barcodes that together function as a single barcode. For example, a polynucleotide barcode can include two or more polynucleotide sequences (e.g., sub-barcodes) that are separated by one or more non-barcode sequences.

### (ii) Nucleic Acid and Nucleotide

The terms “nucleic acid” and “nucleotide” are intended to be consistent with their use in the art and to include unmodified naturally-occurring species or functional analogs thereof. Particularly useful functional analogs of nucleic acids are capable of hybridizing to a nucleic acid in a sequence-specific fashion (e.g., capable of hybridizing to two nucleic acids such that ligation can occur between the two hybridized nucleic acids) or are capable of being used as a template for replication of a particular nucleotide sequence. Unmodified nucleic acids generally have a backbone containing phosphodiester bonds. An analog structure can have an alternate backbone linkage. Unmodified nucleic acids generally have a deoxyribose sugar (e.g., found in deoxyribonucleic acid (DNA)) or a ribose sugar (e.g. found in ribonucleic acid (RNA)).

A nucleic acid can contain nucleotides having any of a variety of analogs of the sugar moieties. A nucleic acid can include native or non-native nucleotides. In this regard, a native deoxyribonucleic acid can have one or more bases selected from the group consisting of adenine (A), thymine (T), cytosine (C), or guanine (G), and a ribonucleic acid can have one or more bases selected from the group consisting of uracil (U), adenine (A), cytosine (C), or guanine (G).

(iii) Probe and Target

A “probe” or a “target,” when used in reference to a nucleic acid or sequence of a nucleic acids, is intended as a semantic identifier for the nucleic acid or sequence in the context of a method or composition, and does not limit the structure or function of the nucleic acid or sequence beyond what is expressly indicated.

### (iv) Oligonucleotide and Polynucleotide

The terms “oligonucleotide” and “polynucleotide” are used interchangeably to refer to a single-stranded multimer of nucleotides from about 2 to about 500 nucleotides in length. Oligonucleotides can be synthetic, made enzymatically (e.g., via polymerization), or using a “split-pool” method. Oligonucleotides can include ribonucleotide monomers (e.g., can be oligoribonucleotides) and/or deoxyribonucleotide monomers (e.g., oligodeoxyribonucleotides). In some examples, oligonucleotides can include a combination of both deoxyribonucleotide monomers and ribonucleotide monomers in the oligonucleotide (e.g., random or ordered combination of deoxyribonucleotide monomers and ribonucleotide monomers). An oligonucleotide can be 4 to 10, 10 to 20, 21 to 30, 31 to 40, 41 to 50, 51 to 60, 61 to 70, 71 to 80, 80 to 100, 100 to 150, 150 to 200, 200 to 250, 250 to 300, 300 to 350, 350 to 400, or 400-500 nucleotides in length, for example. Oligonucleotides can include one or more functional moieties that are attached (e.g., covalently or non-covalently) to the multimer structure. For example, an oligonucleotide can include one or more detectable labels (e.g., a radioisotope or fluorophore).

### (v) Hybridizing, Hybridize, Annealing, and Anneal

The terms “hybridizing,” “hybridize,” “annealing,” and “anneal” are used interchangeably in this disclosure, and refer to the pairing of substantially complementary or complementary nucleic acid sequences within two different molecules. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

### (vi) Primer

A “primer” is a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence.

(vii) Primer Extension

A “primer extension” refers to any method where two nucleic acid sequences become linked (e.g., hybridized) by an overlap of their respective terminal complementary nucleic acid sequences (e.g., for example, 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

(viii) Nucleic Acid Extension

A “nucleic acid extension” generally involves incorporation of one or more nucleic acids (e.g., A, G, C, T, U, nucleotide analogs, or derivatives thereof) into a molecule (such as, but not limited to, a nucleic acid sequence) in a template-dependent manner, such that consecutive nucleic acids are incorporated by an enzyme (such as a polymerase or reverse transcriptase), thereby generating a newly synthesized nucleic acid molecule. For example, a primer that hybridizes to a complementary nucleic acid sequence can be used to synthesize a new nucleic acid molecule by using the complementary nucleic acid sequence as a template for nucleic acid synthesis. Similarly, a 3′ polyadenylated tail of an mRNA transcript that hybridizes to a poly (dT) sequence can be used as a template for single-strand synthesis of a corresponding cDNA molecule.

### (ix) PCR Amplification

A “PCR amplification” refers to the use of a polymerase chain reaction (PCR) to generate copies of genetic material, including DNA and RNA sequences. Suitable reagents and conditions for implementing PCR are described, for example, in U.S. Pat. Nos. 4,683,202, 4,683,195, 4,800,159, 4,965,188, and 5,512,462, the entire contents of each of which are incorporated herein by reference. In a typical PCR amplification, the reaction mixture includes the genetic material to be amplified, an enzyme, one or more primers that are employed in a primer extension reaction, and reagents for the reaction. The oligonucleotide primers are of sufficient length to provide for hybridization to complementary genetic material under annealing conditions. The length of the primers generally depends on the length of the amplification domains, but will typically be at least 4 bases, at least 5 bases, at least 6 bases, at least 8 bases, at least 9 bases, at least 10 base pairs (bp), at least 11 bp, at least 12 bp, at least 13 bp, at least 14 bp, at least 15 bp, at least 16 bp, at least 17 bp, at least 18 bp, at least 19 bp, at least 20 bp, at least 25 bp, at least 30 bp, at least 35 bp, and can be as long as 40 bp or longer, where the length of the primers will generally range from 18 to 50 bp. The genetic material can be contacted with a single primer or a set of two primers (forward and reverse primers), depending upon whether primer extension, linear or exponential amplification of the genetic material is desired.

In some embodiments, the PCR amplification process uses a DNA polymerase enzyme. The DNA polymerase activity can be provided by one or more distinct DNA polymerase enzymes. In certain embodiments, the DNA polymerase enzyme is from a bacterium, e.g., the DNA polymerase enzyme is a bacterial DNA polymerase enzyme. For instance, the DNA polymerase can be from a bacterium of the genus Escherichia, Bacillus, Thermophilus, or Pyrococcus.

Suitable examples of DNA polymerases that can be used include, but are not limited to: E. coli DNA polymerase I, Bsu DNA polymerase, Bst DNA polymerase, Taq DNA polymerase, VENT™ DNA polymerase, DEEPVENT™ DNA polymerase, LongAmp® Taq DNA polymerase, LongAmp® Hot Start Taq DNA polymerase, Crimson LongAmp® Taq DNA polymerase, Crimson Taq DNA polymerase, OneTaq® DNA polymerase, OneTaq® Quick-Load® DNA polymerase, Hemo KlenTaq® DNA polymerase, REDTaq® DNA polymerase, Phusion® DNA polymerase, Phusion® High-Fidelity DNA polymerase, Platinum Pfx DNA polymerase, AccuPrime Pfx DNA polymerase, Phi29 DNA polymerase, Klenow fragment, Pwo DNA polymerase, Pfu DNA polymerase, T4 DNA polymerase and T7 DNA polymerase enzymes.

The term “DNA polymerase” includes not only naturally-occurring enzymes but also all modified derivatives thereof, including also derivatives of naturally-occurring DNA polymerase enzymes. For instance, in some embodiments, the DNA polymerase can have been modified to remove 5′-3′ exonuclease activity. Sequence-modified derivatives or mutants of DNA polymerase enzymes that can be used include, but are not limited to, mutants that retain at least some of the functional, e.g. DNA polymerase activity of the wild-type sequence. Mutations can affect the activity profile of the enzymes, e.g. enhance or reduce the rate of polymerization, under different reaction conditions, e.g. temperature, template concentration, primer concentration, etc. Mutations or sequence-modifications can also affect the exonuclease activity and/or thermostability of the enzyme.

In some embodiments, PCR amplification can include reactions such as, but not limited to, a strand-displacement amplification reaction, a rolling circle amplification reaction, a ligase chain reaction, a transcription-mediated amplification reaction, an isothermal amplification reaction, and/or a loop-mediated amplification reaction.

In some embodiments, PCR amplification uses a single primer that is complementary to the 3′ tag of target DNA fragments. In some embodiments, PCR amplification uses a first and a second primer, where at least a 3′ end portion of the first primer is complementary to at least a portion of the 3′ tag of the target nucleic acid fragments, and where at least a 3′ end portion of the second primer exhibits the sequence of at least a portion of the 5′ tag of the target nucleic acid fragments. In some embodiments, a 5′ end portion of the first primer is non-complementary to the 3′ tag of the target nucleic acid fragments, and a 5′ end portion of the second primer does not exhibit the sequence of at least a portion of the 5′ tag of the target nucleic acid fragments. In some embodiments, the first primer includes a first universal sequence and/or the second primer includes a second universal sequence.

In some embodiments (e.g., when the PCR amplification amplifies captured DNA), the PCR amplification products can be ligated to additional sequences using a DNA ligase enzyme. The DNA ligase activity can be provided by one or more distinct DNA ligase enzymes. In some embodiments, the DNA ligase enzyme is from a bacterium, e.g., the DNA ligase enzyme is a bacterial DNA ligase enzyme. In some embodiments, the DNA ligase enzyme is from a virus (e.g., a bacteriophage). For instance, the DNA ligase can be T4 DNA ligase. Other enzymes appropriate for the ligation step include, but are not limited to, Tth DNA ligase, Taq DNA ligase, Thermococcus sp. (strain 9oN) DNA ligase (9oNTM DNA ligase, available from New England Biolabs, Ipswich, Mass.), and Ampligase™ (available from Epicentre Biotechnologies, Madison, Wis.). Derivatives, e.g. sequence-modified derivatives, and/or mutants thereof, can also be used.

In some embodiments, genetic material is amplified by reverse transcription polymerase chain reaction (RT-PCR). The desired reverse transcriptase activity can be provided by one or more distinct reverse transcriptase enzymes, suitable examples of which include, but are not limited to: M-MLV, MuLV, AMV, HIV, ArrayScript™, MultiScribe™ ThermoScript™, and SuperScript® I, II, III, and IV enzymes. “Reverse transcriptase” includes not only naturally occurring enzymes, but all such modified derivatives thereof, including also derivatives of naturally-occurring reverse transcriptase enzymes.

In addition, reverse transcription can be performed using sequence-modified derivatives or mutants of M-MLV, MuLV, AMV, and HIV reverse transcriptase enzymes, including mutants that retain at least some of the functional, e.g. reverse transcriptase, activity of the wild-type sequence. The reverse transcriptase enzyme can be provided as part of a composition that includes other components, e.g. stabilizing components that enhance or improve the activity of the reverse transcriptase enzyme, such as RNase inhibitor(s), inhibitors of DNA-dependent DNA synthesis, e.g. actinomycin D. Many sequence-modified derivative or mutants of reverse transcriptase enzymes, e.g. M-MLV, and compositions including unmodified and modified enzymes are commercially available, e.g. ArrayScript™, MultiScribe™ ThermoScript™, and SuperScript® I, II, III, and IV enzymes.

Certain reverse transcriptase enzymes (e.g. Avian Myeloblastosis Virus (AMV) Reverse Transcriptase and Moloney Murine Leukemia Virus (M-MuLV, MMLV) Reverse Transcriptase) can synthesize a complementary DNA strand using both RNA (cDNA synthesis) and single-stranded DNA (ssDNA) as a template. Thus, in some embodiments, the reverse transcription reaction can use an enzyme (reverse transcriptase) that is capable of using both RNA and ssDNA as the template for an extension reaction, e.g. an AMV or MMLV reverse transcriptase.

In some embodiments, the quantification of RNA and/or DNA is carried out by real-time PCR (also known as quantitative PCR or qPCR), using techniques well known in the art, such as but not limited to “TAQMAN™” or “SYBR®”, or on capillaries (“LightCycler® Capillaries”). In some embodiments, the quantification of genetic material is determined by optical absorbance and with real-time PCR. In some embodiments, the quantification of genetic material is determined by digital PCR. In some embodiments, the genes analyzed can be compared to a reference nucleic acid extract (DNA and RNA) corresponding to the expression (mRNA) and quantity (DNA) in order to compare expression levels of the target nucleic acids.

### (x) Antibody

An “antibody” is a polypeptide molecule that recognizes and binds to a complementary target antigen. Antibodies typically have a molecular structure shape that resembles a Y shape. Naturally-occurring antibodies, referred to as immunoglobulins, belong to one of the immunoglobulin classes IgG, IgM, IgA, IgD, and IgE. Antibodies can also be produced synthetically. For example, recombinant antibodies, which are monoclonal antibodies, can be synthesized using synthetic genes by recovering the antibody genes from source cells, amplifying into an appropriate vector, and introducing the vector into a host to cause the host to express the recombinant antibody. In general, recombinant antibodies can be cloned from any species of antibody-producing animal using suitable oligonucleotide primers and/or hybridization probes. Recombinant techniques can be used to generate antibodies and antibody fragments, including non-endogenous species.

Synthetic antibodies can be derived from non-immunoglobulin sources. For example, antibodies can be generated from nucleic acids (e.g., aptamers), and from non-immunoglobulin protein scaffolds (such as peptide aptamers) into which hypervariable loops are inserted to form antigen binding sites. Synthetic antibodies based on nucleic acids or peptide structures can be smaller than immunoglobulin-derived antibodies, leading to greater tissue penetration.

Antibodies can also include affimer proteins, which are affinity reagents that typically have a molecular weight of about 12-14 kDa. Affimer proteins generally bind to a target (e.g., a target protein) with both high affinity and specificity. Examples of such targets include, but are not limited to, ubiquitin chains, immunoglobulins, and C-reactive protein. In some embodiments, affimer proteins are derived from cysteine protease inhibitors, and include peptide loops and a variable N-terminal sequence that provides the binding site.

Antibodies can also refer to an “epitope binding fragment” or “antibody fragment,” which as used herein, generally refers to a portion of a complete antibody capable of binding the same epitope as the complete antibody, albeit not necessarily to the same extent. Although multiple types of epitope binding fragments are possible, an epitope binding fragment typically comprises at least one pair of heavy and light chain variable regions (VH and VL, respectively) held together (e.g., by disulfide bonds) to preserve the antigen binding site, and does not contain all or a portion of the Fc region. Epitope binding fragments of an antibody can be obtained from a given antibody by any suitable technique (e.g., recombinant DNA technology or enzymatic or chemical cleavage of a complete antibody), and typically can be screened for specificity in the same manner in which complete antibodies are screened. In some embodiments, an epitope binding fragment comprises an F(ab′)₂ fragment, Fab′ fragment, Fab fragment, Fd fragment, or Fv fragment. In some embodiments, the term “antibody” includes antibody-derived polypeptides, such as single chain variable fragments (scFv), diabodies or other multimeric scFvs, heavy chain antibodies, single domain antibodies, or other polypeptides comprising a sufficient portion of an antibody (e.g., one or more complementarity determining regions (CDRs)) to confer specific antigen binding ability to the polypeptide.

### (xi) Label, Detectable Label, and Optical Label

The terms “detectable label,” “optical label,” and “label” are used interchangeably herein to refer to a directly or indirectly detectable moiety that is associated with (e.g., conjugated to) a molecule to be detected, e.g., a probe for in situ assay. The detectable label can be directly detectable by itself (e.g., radioisotope labels or fluorescent labels) or, in the case of an enzymatic label, can be indirectly detectable, e.g., by catalyzing chemical alterations of a substrate compound or composition, which substrate compound or composition is directly detectable. Detectable labels can be suitable for small scale detection and/or suitable for high-throughput screening. As such, suitable detectable labels include, but are not limited to, radioisotopes, fluorophores, chemiluminescent compounds, bioluminescent compounds, and dyes.

The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties. In some embodiments, the detectable label is bound to a feature or to a probe associated with a feature. For example, detectably labelled features can include a fluorescent, a colorimetric, or a chemiluminescent label attached to a bead (see, for example, Rajeswari et al., J. Microbiol Methods 139:22-28, 2017, and Forcucci et al., J. Biomed Opt. 10:105010, 2015, the entire contents of each of which are incorporated herein by reference).

In some embodiments, a plurality of detectable labels can be attached to a feature, probe, or composition to be detected. For example, detectable labels can be incorporated during nucleic acid polymerization or amplification (e.g., Cy5®-labelled nucleotides, such as Cy5®-dCTP). Any suitable detectable label can be used. In some embodiments, the detectable label is a fluorophore. For example, the fluorophore can be from a group that includes: 7-AAD (7-Aminoactinomycin D), Acridine Orange (+DNA), Acridine Orange (+RNA), Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA/AMCA-X, 7-Aminoactinomycin D (7-AAD), 7-Amino-4-methylcoumarin, 6-Aminoquinoline, Aniline Blue, ANS, APC-Cy7, ATTO-TAG™ CBQCA, ATTO-TAG™ FQ, Auramine O-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1™, Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GeneBLAzer™), CFP (Cyan Fluorescent Protein), CFP/YFP FRET, Chromomycin A3, C1-NERF (low pH), CPM, 6-CR 6G, CTC Formazan, Cy2®, Cy3®, Cy3.5@, Cy5®, Cy5.5®, Cy7®, Cychrome (PE-Cy5), Dansylamine, Dansyl cadaverine, Dansylchloride, DAPI, Dapoxyl, DCFH, DHR, DiA (4-Di-16-ASP), DiD (DilC18(5)), DIDS, Dil (DilC18(3)), DiO (DiOC18(3)), DiR (DilC18(7)), Di-4 ANEPPS, Di-8 ANEPPS, DM-NERF (4.5-6.5 pH), DsRed (Red Fluorescent Protein), EBFP, ECFP, EGFP, ELF®-97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX®, Fluoro-Gold™ (high pH), Fluoro-Gold™ (low pH), Fluoro-Jade, FM® 1-43, Fura-2 (high calcium), Fura-2/BCECF, Fura Red™ (high calcium), Fura Red™/Fluo-3, GeneBLAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™-1/JO-PRO™-1, LDS 751 (+DNA), LDS 751 (+RNA), LOLO™-1/LO-PRO™-1, Lucifer Yellow, LysoSensor™ Blue (pH 5), LysoSensor™ Green (pH 5), LysoSensor™ Yellow/Blue (pH 4.2), LysoTracker® Green, LysoTracker® Red, LysoTracker® Yellow, Mag-Fura-2, Mag-Indo-1, Magnesium Green™, Marina Blue®, 4-Methylumbelliferone, Mithramycin, MitoTracker® Green, MitoTracker® Orange, MitoTracker® Red, NBD (amine), Nile Red, Oregon Green® 488, Oregon Green® 500, Oregon Green® 514, Pacific Blue, PBF1, PE (R-phycoerythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™-1/PO-PRO™-1, POPO™-3/PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red), Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARF®-1 (low pH), Sodium Green™, SpectrumAqua®, SpectrumGreen® #1, SpectrumGreen® #2, SpectrumOrange®, SpectrumRed®, SYTO® 11, SYTO® 13, SYTO® 17, SYTO® 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X, Texas Red®-X (NHS Ester), Thiadicarbocyanine, Thiazole Orange, TOTO®-1/TO-PRO®-1, TOTO®-3/TO-PRO®-3, TO-PRO®-5, Tri-color (PE-Cy5), TRITC (Tetramethylrhodamine), TruRed (PerCP-Cy5.5), WW 781, X-Rhodamine (XRITC), Y66F, Y66H, Y66W, YFP (Yellow Fluorescent Protein), YOYO@-1/YO-PRO@-1, YOYO@-3/YO-PRO@-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 550, ATTO 565, ATTO Rhol01, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester).

As mentioned above, in some embodiments, a detectable label is or includes a luminescent or chemiluminescent moiety. Common luminescent/chemiluminescent moieties include, but are not limited to, peroxidases such as horseradish peroxidase (HRP), soybean peroxidase (SP), alkaline phosphatase, and luciferase. These protein moieties can catalyze chemiluminescent reactions given the appropriate substrates (e.g., an oxidizing reagent plus a chemiluminescent compound. A number of compound families are known to provide chemiluminescence under a variety of conditions. Non-limiting examples of chemiluminescent compound families include 2,3-dihydro-1,4-phthalazinedione luminol, 5-amino-6,7,8-trimethoxy- and the dimethylamino[ca]benz analog. These compounds can luminesce in the presence of alkaline hydrogen peroxide or calcium hypochlorite and base. Other examples of chemiluminescent compound families include, e.g., 2,4,5-triphenylimidazoles, para-dimethylamino and—methoxy substituents, oxalates such as oxalyl active esters, p-nitrophenyl, N-alkyl acridinum esters, luciferins, lucigenins, or acridinium esters. In some embodiments, a detectable label is or includes a metal-based or mass-based label. For example, small cluster metal ions, metals, or semiconductors may act as a mass code. In some examples, the metals can be selected from Groups 3-15 of the periodic table, e.g., Y, La, Ag, Au, Pt, Ni, Pd, Rh, Ir, Co, Cu, Bi, or a combination thereof.

Exemplary embodiments of the present disclosure include:

1A. A method for analyzing a biological sample, comprising:
(a) contacting the biological sample with a reaction mixture comprising one or more modified nucleotides or nucleotide analogs comprising a modified nucleotide or nucleotide analog having a hydrophobic modification,
(b) performing rolling circle amplification (RCA) of a circular nucleic acid template in the biological sample using a polymerase, thereby generating an RCA product incorporating the one or more modified nucleotides or nucleotide analogs, wherein the RCA product is not crosslinked via the one or more modified nucleotides or nucleotide analogs incorporated into the RCA product, and.
(c) detecting the RCA product not crosslinked via the one or more modified nucleotides or nucleotide analogs at a location in the biological sample.
   2A. The method of embodiment 1A, wherein the hydrophobic modification is a base modification.
   3A. The method of embodiment 1A or 2A, wherein the hydrophobic modification comprises a carbon chain and/or a hydrocarbon ring.
   4A. The method of any of embodiments 1A-3A, wherein the hydrophobic modification comprises a triple bond.
   5A. The method of any of embodiments 1A-4A, wherein the hydrophobic modification comprises a vinyl or ethynyl group.
   6A. The method of any of embodiments 1A-5A, wherein the modified nucleotide or nucleotide analog comprising the hydrophobic modification is an ethynyl-dUTP or a vinyl-dUTP.
   7A. The method of embodiment 6A, wherein the modified nucleotide or nucleotide analog comprising the hydrophobic modification is a 5-ethynyl-dUTP or a 5-vinyl-dUTP.
   8A. The method of any of embodiments 1A-7A, wherein the diameter of the RCA product generated using the one or more modified nucleotides or nucleotide analogs is smaller than a reference RCA product produced using the same template without including the one or more modified nucleotides or nucleotide analogs in the reaction mixture.
   9A. The method of any of embodiments 1A-8A, wherein the modified nucleotide or nucleotide analog comprising the hydrophobic modification is added to the biological sample at a concentration of at least 1 μM, at least 1.25 μM, at least 2.5 μM, at least 5 μM, at least 10 μM, at least 40 μM, at least 80 μM, or at least 100 μM.
   10A. The method of any of embodiments 1A-9A, wherein the modified nucleotide or nucleotide analog is a modified dUTP and the ratio of the modified dUTP to an unmodified dUTP or dTTP in the reaction mixture is between about 80:20 and about 1:99, optionally wherein the ratio of the modified dUTP to the unmodified dUTP or dTTP in the reaction mixture is between about 80:20 and about 40:60.
   11A. The method of any of embodiments 1A-10A, wherein the modified nucleotide or nucleotide analog comprising the hydrophobic modification is added to the biological sample at a concentration of about 50 μM to about 100 μM, optionally wherein the modified nucleotide or nucleotide analog comprising the hydrophobic modification is added to the biological sample at a concentration of about 80 μM to about 100 μM.
   12A. The method of any of embodiments 1A-11A, wherein the median diameter of an RCA product generated using the one or more modified nucleotides or nucleotide analogs is smaller than the median diameter of a reference RCA product produced using the same template without including the one or more modified nucleotides or nucleotide analogs in the reaction mixture.
   13A. The method of any of embodiments 1A-12A, wherein the median diameter of an RCA product generated using the one or more modified nucleotides or nucleotide analogs is less than 500 nm.
   14A. The method of any of embodiments 1A-13A, wherein the median diameter of an RCA product generated using the one or more modified nucleotides or nucleotide analogs is no more than 90% or no more than 80% of the median diameter of a reference RCA product produced using the same template without including the one or more modified nucleotides or nucleotide analogs in the reaction mixture.
   15A. The method of any of embodiments 1A-14A, wherein the average intensity, average signal-to-noise ratio, and/or density of an RCA product incorporating the one or more modified nucleotides or nucleotide analogs is not significantly different from the average intensity, average signal-to-noise ratio, and/or density of a reference RCA product produced using the same template without including the one or more modified nucleotides or nucleotide analogs in the reaction mixture.
   16A. The method of any of embodiments 1A-15A, wherein the incorporation of the one or more modified nucleotides or nucleotide analogs into the RCA product increases overall hydrophobicity of the RCA product.
   17A. The method of any of embodiments 1A-16A, wherein the incorporation of the one or more modified nucleotides or nucleotide analogs into the RCA product promotes base stacking interactions among nucleotides in the RCA product.
   18A. The method of any of embodiments 1A-17A, wherein: the one or more modified nucleotides or nucleotide analogs incorporated into the RCA product do not comprise an amine; and/or the one or more modified nucleotides or nucleotide analogs incorporated into the RCA product do not comprise a detectable label, optionally wherein the detectable label is a fluorophore.
   19A. The method of any of embodiments 1A-18A, comprising in (c), contacting the biological sample with a nucleic acid probe that hybridizes to the RCA product, optionally wherein the nucleic acid probe comprises a detectable label, and optionally wherein the detectable label is a fluorophore.
   20A. The method of embodiment 19A, wherein the nucleic acid probe is an intermediate probe, and the method comprises contacting the biological sample with a detectably labeled probe that hybridizes to the intermediate probe.
   21A. A method for analyzing a biological sample, comprising:
(a) contacting the biological sample with a reaction mixture comprising one or more nucleotides or nucleotide analogs,
(b) performing rolling circle amplification (RCA) of a circular nucleic acid template in the biological sample using a polymerase, thereby generating an RCA product,
wherein the one or more nucleotides or nucleotide analogs comprise:
   (i) a non-incorporable nucleotide or analog thereof that is not incorporated by the polymerase, and/or
   (ii) an incorporable nucleotide or nucleotide analog that is configured to be incorporated by the polymerase at a slower rate than a corresponding nucleoside triphosphate, and
(c) detecting the RCA product which is not crosslinked via nucleotide(s) or nucleotide analog(s) at a location in the biological sample.
   22A. The method of embodiment 21A, wherein in (c), the RCA product is not crosslinked, via a nucleotide or nucleotide analog incorporated into the RCA product, to the RCA product itself, to another molecule in the biological sample, or to a matrix embedding the biological sample.
   23A. The method of embodiment 21A or 22A, wherein: the one or more nucleotides or nucleotide analogs incorporated into the RCA product do not comprise an amine; and/or the one or more nucleotides or nucleotide analogs incorporated into the RCA product do not comprise a detectable label, optionally wherein the detectable label is a fluorophore.
   24A. The method of any of embodiments 21A-23A, comprising in (c), contacting the biological sample with a nucleic acid probe that hybridizes to the RCA product, optionally wherein the nucleic acid probe comprises a detectable label, and optionally wherein the detectable label is a fluorophore, optionally wherein the nucleic acid probe is an intermediate probe, and the method comprises contacting the biological sample with a detectably labeled probe that hybridizes to the intermediate probe.
   25A. The method of any of embodiments 21A-24A, wherein the presence of one or more modified nucleotides or nucleotide analogs in the reaction mixture decreases the polymerization rate of the polymerase and/or the size of the RCA product as compared to a reference reaction mixture without the one or more modified nucleotides or nucleotide analogs, optionally wherein the reference reaction mixture comprises only unmodified dATP, dTTP and/or dUTP, dCTP, and dGTP.
   26A. The method of any of embodiments 1A-25A, comprising crosslinking the RCA product to itself or to another molecule at one or more nucleotide resides other than: (i) the modified nucleotide or nucleotide analog residue having the hydrophobic modification, and/or (ii) the incorporable nucleotide or nucleotide analog incorporated into the RCA product, wherein the crosslinking is performed prior to and/or after detecting the RCA product.
   27A. A method for analyzing a biological sample, comprising:
(a) contacting the biological sample with a circular probe or circularizable probe or probe set comprising one or more modified nucleotide or nucleotide analog residues, wherein the circular probe or circularizable probe or probe set comprises a hybridization region that hybridizes to a target nucleic acid in the biological sample, and wherein the one or more modified nucleotide or nucleotide analog residues are outside the hybridization region,
(b) using a polymerase to perform rolling circle amplification (RCA) of the circular probe or of a circularized probe generated from the circularizable probe or probe set, thereby generating an RCA product,
wherein the presence of the one or more modified nucleotide or nucleotide analog residues decreases the polymerization rate of the polymerase on the circular or circularized probe and/or the size of the RCA product as compared to a reference circular template without the one or more modified nucleotide or nucleotide analog residues, and
(c) detecting the RCA product at a location in the biological sample.
   28A. The method of embodiment 27A, wherein the one or more modified nucleotide or nucleotide analog residues comprise modified deoxyribonucleotide (DNA) or DNA analog residues and/or modified ribonucleotide (RNA) or RNA analog residues.
   29A. The method of embodiment 27A or 28A, wherein the one or more modified nucleotide or nucleotide analog residues do not comprise an amine and/or a detectable label, optionally wherein the detectable label is a fluorophore.
   30A. A method for analyzing a biological sample, comprising:
(a) contacting the biological sample with (i) a first circular probe or circularizable probe or probe set comprising one or more modified nucleotide or nucleotide analog residues, wherein the first circular probe or circularizable probe or probe set hybridizes to a first target nucleic acid in the biological sample, and (ii) a second circular probe or circularizable probe or probe set, wherein the second circular probe or circularizable probe or probe set hybridizes to a second target nucleic acid in the biological sample,
(b) using a polymerase to perform rolling circle amplification (RCA) of the first circular probe or of a first circularized probe generated from the first circularizable probe or probe set, thereby generating a first RCA product, and
(c) using a polymerase to perform rolling circle amplification (RCA) of the second circular probe or of a second circularized probe generated from the second circularizable probe or probe set, thereby generating a second RCA product,
wherein the presence of the one or more modified nucleotide or nucleotide analog residues in the first circular or circularized probe decreases the polymerization rate of the polymerase and/or the size of the RCA product using the first circular or circularized probe as template as compared to a reference circular template without the one or more modified nucleotide or nucleotide analog residues; and/or wherein the polymerization rate of the polymerase and/or the size of the RCA product using the first circular or circularized probe as template is less than the polymerization rate of the polymerase and/or the size of the RCA product using the second circular or circularized probe as template
   1. A method for analyzing a biological sample, comprising:
(a) contacting the biological sample with a circular probe or circularizable probe or probe set that hybridizes to a target nucleic acid in the biological sample, wherein the circular probe or a circularized probe generated from the circularizable probe or probe set comprises one or more modified nucleotide or nucleotide analog residues, and the one or more modified nucleotide or nucleotide analog residues are outside a region that hybridizes to the target nucleic acid,
(b) using a polymerase to perform rolling circle amplification (RCA) of the circular probe or the circularized probe, thereby generating an RCA product,
wherein the presence of the one or more modified nucleotide or nucleotide analog residues in the circular probe or circularized probe decreases the polymerization rate of the polymerase using the circular probe or circularized probe as template as compared to a reference circular template without the one or more modified nucleotide or nucleotide analog residues.
   2. The method of embodiment 1, wherein the one or more modified nucleotide or nucleotide analog residues comprise a sugar modified nucleotide.
   3. The method of embodiment 1 or 2, wherein the one or more modified nucleotide or nucleotide analog residues comprise a C2′ modified nucleotide.
   4. The method of any of embodiments 1-3, wherein the one or more modified nucleotide or nucleotide analog residues have a C3′ endo pucker conformation.
   5. The method of any of embodiments 1-4, wherein the one or more modified nucleotide or nucleotide analog residues comprise one or more modified ribonucleotide residues.
   6. The method of any of embodiments 1-5, wherein the circular probe or circularized probe does not comprise an unmodified ribonucleotide residue.
   7. The method of any of embodiments 1-5, wherein the circular probe or circularized probe comprises one or more unmodified ribonucleotide residues.
   8. The method of any of embodiments 1-7, wherein the one or more modified nucleotide or nucleotide analog residues are selected from the group consisting of 2′-O-methyl ribonucleic acid (2′-OMeRNA), locked nucleic acid (LNA), 2′-fluoro ribonucleic acid (2′-F RNA), and combinations thereof.
   9. The method of any of embodiments 1-8, wherein the one or more modified nucleotide or nucleotide analog residues comprise 2′-OMeRNA.
   10. The method of any of embodiments 1-9, wherein the one or more modified nucleotide or nucleotide analog residues comprise modified deoxyribonucleotide residues.
   11. The method of any of embodiments 1-10, wherein the circular probe or circularized probe is primarily composed of unmodified deoxyribonucleotide residues.
   12. The method of any of embodiments 1-11, wherein the circular probe or circularized probe comprises no more than 20%, no more than 10%, no more than 5%, or no more than 1% of modified nucleotide or nucleotide analog residues and/or unmodified ribonucleotide residues.
   13. The method of any of embodiments 1-12, wherein the circular probe or circularized probe comprises no more than two, no more than three, no more than four, or no more than five consecutive modified nucleotide or nucleotide analog residues and/or unmodified ribonucleotide residues.
   14. The method of any of embodiments 1-13, wherein the one or more modified nucleotide or nucleotide analog residues comprise one or more triazole or thiol-based linkages instead of a phosphodiester linkage.
   15. The method of embodiment 14, wherein the circular probe or circularized probe comprises one or more phosphorothioate linkages between residues.
   16. The method of embodiment 14 or 15, wherein the circular probe or circularized probe comprises one or more thiophosphate linkages between residues.
   17. The method of any of embodiments 1-16, wherein the method comprises contacting the biological sample with a circularizable probe or probe set comprising one or more thiol linkages, and the one or more thiol linkages are not located at a 5′ or 3′ end of the circularizable probe or probe set.
   18. The method of any of embodiments 1-17, wherein the circular probe or circularized probe comprises two or more triazole linkages.
   19. The method of any of embodiments 1-18, wherein the circular probe or circularized probe comprises two or more thiol linkages.
   20. The method of embodiment 18 or 19, wherein at least two of the two or more triazole linkages or the two or more thiol linkages are separated by fewer than 100 nucleotides.
   21. The method of any of embodiments 1-20, wherein the circular probe or circularizable probe or probe set is a first circular probe or circularizable probe or probe set, the circularized probe is a first circularized probe, and the target nucleic acid is a first target nucleic acid, and the method further comprises contacting the biological sample with a second circular or circularizable probe or probe set, wherein the second circular or circularizable probe or probe set hybridizes to a second target nucleic acid in the biological sample, and
using a polymerase to perform rolling circle amplification (RCA) of the second circular probe or of a second circularized probe generated from the second circularizable probe or probe set, thereby generating a second RCA product.
   22. The method of embodiment 21, wherein the second circular probe or circularized probe does not comprise modified nucleotide or nucleotide analog residues, or wherein the second circular probe or circularized probe comprises fewer modified nucleotide or nucleotide analog residues than the first circular probe or circularized probe.
   23. The method of embodiment 21 or 22, wherein the second circular probe or circularized probe comprises different modified nucleotide or nucleotide analog residues than the first circular probe or circularized probe.
   24. The method of any of embodiments 21-23, wherein the polymerization rate of the polymerase using the first circular or circularized probe as template is slower than the polymerization rate of the polymerase using the second circular or circularized probe as template.
   25. The method of any of embodiments 1-24, wherein the method comprises contacting the biological sample with a reaction mixture comprising one or more nucleotides or analogs thereof, wherein the one or more nucleotides or analogs thereof comprise:
(i) a non-incorporable nucleotide or analog thereof that is configured to bind transiently to the polymerase but not be incorporated by the polymerase, and/or
(ii) an incorporable nucleotide or analog thereof that is configured to be incorporated by the polymerase at a slower rate than a corresponding nucleoside triphosphate.
   26. A method for analyzing a biological sample, comprising:
(a) contacting the biological sample with a reaction mixture comprising one or more nucleotides or analogs thereof, and
(b) performing rolling circle amplification (RCA) of a circular nucleic acid template in the biological sample using a polymerase, thereby generating an RCA product,
wherein the one or more nucleotides or analogs thereof comprise:
(i) a non-incorporable nucleotide or analog thereof that binds transiently to the polymerase but is not incorporated by the polymerase, and/or
(ii) an incorporable nucleotide or analog thereof that is configured to be incorporated by the polymerase at a slower rate than a corresponding nucleoside triphosphate.
   27. The method of embodiment 25 or 26, wherein the one or more nucleotides or analogs thereof comprise a non-incorporable nucleotide or analog thereof that binds transiently to the polymerase but is not incorporated by the polymerase, and an incorporable nucleotide or analog thereof that is configured to be incorporated by the polymerase at a slower rate than a corresponding nucleoside triphosphate.
   28. The method of any of embodiments 25-27, wherein the mean open time of the polymerase during polymerization is greater for incorporation of the incorporable nucleotide or analog thereof than for incorporation of a corresponding naturally-occurring nucleoside triphosphate.
   29. The method of embodiment 28, wherein the mean open time of the polymerase during polymerization for incorporation of the incorporable nucleotide is at least 125%, 150%, 200%, 225%, or 250% of the mean open time for incorporation of a corresponding nucleoside triphosphate.
   30. The method of any of embodiments 25-29, wherein the incorporable nucleotide or analog thereof comprises an alpha-thiol nucleotide, an alkyne modified nucleotide, or an azide modified nucleotide.
   31. The method of any of embodiments 25-30, wherein the incorporable nucleotide or analog thereof comprises a diphosphate nucleotide.
   32. The method of embodiment 30, wherein the method does not comprise contacting the biological sample with deoxyribose nucleoside triphosphates.
   33. The method of any of embodiments 25-32, wherein no more than 50%, 40%, 30%, 20%, 10%, 5% or 1% of the nucleotides or analogs thereof in the reaction mixture are the non-incorporable nucleotide or analog thereof and/or the incorporable nucleotide or analog thereof.
   34. The method of any of embodiments 25-33, wherein the non-incorporable nucleotide or analog thereof comprises a monophosphate nucleotide.
   35. The method of any of embodiments 25-34, wherein the non-incorporable nucleotide or analog thereof is dissociable from the polymerase.
   36. The method of any of embodiments 25-35, wherein the mean rate of polymerization by the polymerase in the rolling circle amplification is inversely correlated with the concentration of the one or more incorporable or non-incorporable nucleotides or analogs thereof in the reaction mixture.
   37. The method of any of embodiments 1-36, wherein the mean rate of polymerization by the polymerase in the rolling circle amplification is less than 2280 nt/min, less than 2000 nt/min, less than 1500 nt/min, less than 1250 nt/min, less than 1000 nt/min, less than 750 nt/min, less than 500 nt/min, or less than 250 nt/min.
   38. A method for analyzing a biological sample, comprising:
(a) contacting the biological sample with a reaction mixture comprising one or more nucleotide analogs comprising a hydrophobic modification, and
(b) performing rolling circle amplification (RCA) of a circular nucleic acid template in the biological sample using a polymerase, thereby generating an RCA product incorporating the one or more nucleotide analogs,
wherein the RCA product comprising the one or more nucleotide analogs is smaller than a reference RCA product not comprising the one or more nucleotide analogs.
   39. The method of embodiment 38, wherein the hydrophobic modification is a base modification.
   40. The method of embodiment 38 or 39, wherein the hydrophobic modification comprises a carbon chain and/or a hydrocarbon ring.
   41. The method of any of embodiments 38-40, wherein the hydrophobic modification comprises a triple bond.
   42. The method of any of embodiments 38-41, wherein the hydrophobic modification comprises a vinyl or ethynyl group.
   43. The method of any of embodiments 38-42, wherein the nucleotide analog comprising the hydrophobic modification is an ethynyl-dUTP or a vinyl-dUTP.
   44. The method of any of embodiments 38-43, wherein the nucleotide analog comprising the hydrophobic modification is a 5-ethynyl-dUTP or a 5-vinyl-dUTP.
   45. The method of any of embodiments 38-44, wherein the diameter of the RCA product generated using the one or more nucleotide analogs is smaller than a reference RCA product produced using the same circular nucleic acid template without including the one or more nucleotide analogs in the reaction mixture.
   46. The method of any of embodiments 38-45, wherein the nucleotide analog comprising the hydrophobic modification is added to the sample at a concentration of at least 1 μM, at least 1.25 M, at least 2.5 μM, at least 5 μM, at least 10 μM, at least 40 μM, at least 80 μM, or at least 100 M.
   47. The method of any of embodiments 38-46, wherein the nucleotide analog is a modified dUTP and the ratio of the modified dUTP to an unmodified dUTP or dTTP in the reaction mixture is between about 80:20 and about 1:99, optionally wherein the ratio of the modified dUTP to the unmodified dUTP or dTTP in the reaction mixture is between about 80:20 and about 40:60.
   48. The method of any of embodiments 38-47, wherein the nucleotide analog comprising the hydrophobic modification is added to the sample at a concentration of about 50 μM to about 100 M, optionally wherein the nucleotide analog comprising the hydrophobic modification is added to the sample at a concentration of about 80 μM to about 100 μM.
   49. The method of any of embodiments 38-48, wherein the median diameter of the RCA product having incorporated the one or more nucleotide analogs is less than the median diameter of a reference RCA product generated using the same circular nucleic acid template without including the one or more nucleotide analogs in the reaction mixture.
   50. The method of any of embodiments 38-49, wherein the median diameter of the RCA product incorporating the one or more nucleotide analogs is less than 500 nm.
   51. The method of any of embodiments 38-50, wherein the median diameter of the RCA product incorporating the one or more nucleotide analogs is no more than 90% or no more than 80% of the median diameter of a reference RCA product generated using the same circular nucleic acid template without including the one or more nucleotide analogs in the reaction mixture.
   52. The method of any of embodiments 38-51, wherein the average intensity, average signal-to-noise ratio, and/or density of the RCA product incorporating the one or more nucleotide analogs is not significantly different from the average intensity, average signal-to-noise ratio, and/or density of a reference RCA product generated using the same circular nucleic acid template without including the one or more nucleotide analogs in the reaction mixture, optionally wherein the reference RCA product is generated using the same circular nucleic acid template without including the one or more nucleotide analogs in the reaction mixture, and optionally wherein the total number of nucleotides and nucleotide analogs in the RCA product is not less than the total number of nucleotides in the reference RCA product.
   53. The method of any of embodiments 38-52, wherein the incorporation of the one or more nucleotide analogs into the RCA product increases overall hydrophobicity of the RCA product.
   54. The method of any of embodiments 38-52, wherein the incorporation of the one or more nucleotide analogs into the RCA product promotes base stacking interactions among nucleotides in the RCA product.
   55. The method of any of embodiments 1A-54, wherein the rolling circle amplification is performed at a temperature between 18° C. and 30° C.
   56. The method of embodiment 55, wherein the rolling circle amplification is performed at 30° C.
   57. The method of any of embodiments 1A-56, wherein the RCA product is generated using linear RCA, branched RCA, dendritic RCA, or any combination thereof.
   58. The method of any of embodiments 1A-57, wherein the RCA product is generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst DNA polymerase, rBST DNA polymerase, N29 DNA polymerase, TopoTaq DNA polymerase, T7 RNA polymerase, SP6 RNA polymerase, T3 RNA polymerase, and a variant or derivative of any of the foregoing.
   59. The method of any of embodiments 1A-58, wherein the polymerase is a Phi29 DNA polymerase, a Vent DNA polymerase, or a Bst DNA polymerase.
   60. The method of any of embodiments 1A-59, wherein the polymerase is a Phi29 DNA polymerase.
   61. The method of any of embodiments 1A-60, wherein the RCA product is generated in situ in the biological sample or in a matrix embedding the biological sample or molecules thereof.
   62. The method of any of embodiments 1A-61, wherein the RCA product is crosslinked to one or more other molecules in the biological sample and/or a matrix embedding the biological sample or molecules thereof.
   63. The method of any of embodiments 1A-62, comprising detecting the RCA product in situ in the biological sample or in a matrix embedding the biological sample or molecules thereof.
   64. The method of embodiment 63, wherein detecting the RCA product comprises contacting the biological sample with one or more detectably-labeled probes that directly or indirectly hybridize to one or more barcode sequences comprised by the RCA product.
   65. The method of any of embodiments 1A-64, wherein a signal associated with the RCA product is amplified in situ in the biological sample or in a matrix embedding the biological sample or molecules thereof.
   66. The method of embodiment 65, wherein the signal amplification comprises rolling circle amplification (RCA) of a probe that directly or indirectly binds to the RCA product; hybridization chain reaction (HCR) directly or indirectly on the RCA product; linear oligonucleotide hybridization chain reaction (LO-HCR) directly or indirectly on the RCA product; primer exchange reaction (PER) directly or indirectly on the RCA product; assembly of branched structures directly or indirectly on the RCA product; hybridization of a plurality of detectable probes directly or indirectly on the RCA product, or any combination thereof.
   67. The method of any of embodiments 1A-66, wherein the method further comprises terminating the rolling circle amplification by heat denaturation and/or by contacting the biological sample with a polymerase inhibitor.
   68. The method of embodiment 67, wherein terminating the rolling circle amplification comprises contacting the biological sample with a polymerase inhibitor selected from the group consisting of a pyrophosphate analog, an allosteric inhibitor of the polymerase, a non-catalytic ion that binds to the polymerase, and a chain terminating nucleotide.
   69. The method of any of embodiments 26-68, wherein the circular nucleic acid template is a circular probe or a circularized probe generated from a circularizable probe or probe set, wherein the circular probe or circularizable probe or probe set hybridizes to a target nucleic acid in the biological sample.
   70. The method of any of embodiments 1A-69, wherein the circularized probe is generated from the circularizable probe or probe set using the target nucleic acid as a ligation template.
   71. The method of any of embodiments 1A-70, wherein the circularizable probe set comprises two, three, or more probes.
   72. The method of any of embodiments 1A-71, wherein the circularized probe is generated using enzymatic ligation and/or chemical ligation.
   73. The method of any of embodiments 1A-72, wherein the circularized probe is generated using template dependent ligation and/or template independent ligation.
   74. The method of any of embodiments 1A-73, wherein the circularized probe is generated using a ligase having an RNA-templated DNA ligase activity and/or an RNA-templated RNA ligase activity.
   75. The method of any of embodiments 1A-74, wherein the circularized probe is generated using a ligase selected from the group consisting of a Chlorella virus DNA ligase (also known as PBCV-1 DNA ligase), a T4 RNA ligase, a T4 DNA ligase, and a single-stranded DNA (ssDNA) ligase.
   76. The method of any of embodiments 1A-75, wherein the circularized probe is generated using a PBCV-1 DNA ligase or variant or derivative thereof and/or a T4 RNA ligase 2 (also known as T4 Rnl2) or variant or derivative thereof.
   77. The method of any of embodiments 1A-76, wherein the RCA product comprises one or more barcode sequences or complements thereof.
   78. The method of embodiment 77, wherein the one or more barcode sequences or complements thereof correspond to the target nucleic acid or a portion thereof.
   79. The method of any of embodiments 1A-78, wherein the RCA product comprises between about 10 and about 100, between about 100 and about 1,000, between about 1,000 and about 5,000, between about 5,000 and about 10,000, or more than 10,000 copies of the circular nucleic acid template or the circular or circularized probe.
   80. The method of any of embodiments 1A-79, wherein the RCA product is in the form of a nanoball having a diameter of between about 0.1 μm and about 3 μm, optionally wherein the diameter is between about 0.1 μm and about 0.5 μm, between about 0.5 μm and about 1 μm, between about 0.8 μm and about 1.3 μm, or between about 1 μm and about 1.5 μm.
   81. The method of any of embodiments 1A-80, wherein the RCA product is between about 1 and about 15 kilobases, between about 15 and about 25 kilobases, between about 25 and about 35 kilobases, between about 35 and about 45 kilobases, between about 45 and about 55 kilobases, between about 55 and about 65 kilobases, between about 65 and about 75 kilobases, or more than 75 kilobases in length.
   82. The method of any of embodiments 1A-81, wherein the target nucleic acid comprises DNA and/or RNA, optionally wherein the target nucleic acid is genomic DNA, RNA, mRNA, cDNA, or a reporter oligonucleotide of a labelling agent that directly or indirectly binds to an analyte in the biological sample.
   83. The method of any of embodiments 1A-82, further comprising crosslinking the RCA product to itself, to one or more other molecules in the biological sample, and/or to a matrix embedding the biological sample or molecules thereof, optionally wherein the crosslinking reduces the mobility of the RCA product in the biological sample and/or in the matrix.
   84. The method of any of embodiments 1A-83, wherein the biological sample is a fixed and/or permeabilized biological sample.
   85. The method of any of embodiments 1A-84, wherein the biological sample is a non-homogenized tissue sample or a tissue section.
   86. The method of any of embodiments 1A-85, wherein the biological sample is a formalin-fixed, paraffin-embedded (FFPE) tissue sample, a frozen tissue sample, or a fresh tissue sample.
   87. The method of any of embodiments 1A-86, wherein the biological sample is a tissue slice between about 1 μm and about 50 μm in thickness, optionally wherein the tissue slice is between about 5 μm and about 35 μm in thickness.
   88. The method of any of embodiments 1A-87, wherein the biological sample is crosslinked.
   89. The method of any of embodiments 1A-88, wherein the biological sample is embedded in a matrix, optionally wherein the matrix is a hydrogel.
   90. The method of any of embodiments 1A-89, wherein the biological sample is cleared.
   91. A method for analyzing a biological sample, comprising:
(a) contacting the biological sample with (i) a first circular probe or circularizable probe or probe set comprising one or more modified nucleotide or nucleotide analog residues, wherein the first circular probe or circularizable probe or probe set hybridizes to a first target nucleic acid in the biological sample, and (ii) a second circular probe or circularizable probe or probe set, wherein the second circular probe or circularizable probe or probe set hybridizes to a second target nucleic acid in the biological sample, and
(b) using a polymerase to perform rolling circle amplification (RCA) of the first circular probe or of a first circularized probe generated from the first circularizable probe or probe set, thereby generating a first RCA product, and
using a polymerase to perform rolling circle amplification (RCA) of the second circular probe or of a second circularized probe generated from the second circularizable probe or probe set, thereby generating a second RCA product,
wherein the presence of the one or more modified nucleotide or nucleotide analog residues in the first circular or circularized probe decreases the polymerization rate of the polymerase using the first circular or circularized probe as template as compared to a reference circular template without the one or more modified nucleotide or nucleotide analog residues, and/or
wherein the polymerization rate of the polymerase using the first circular or circularized probe as template is slower than the polymerization rate of the polymerase using the second circular or circularized probe as template.
   92. The method of embodiment 91, wherein the second circular or circularized probe does not comprise modified nucleotide or nucleotide analog residues.
   93. The method of embodiment 91, wherein the second circular or circularized probe comprises fewer modified nucleotide or nucleotide analog residues than the first circular or circularized probe.
   94. The method of any of embodiments 91-93, wherein the first target nucleic acid is more abundant in the biological sample than the second target nucleic acid.
   95. A kit for performing rolling circle amplification, the kit comprising:
(a) a circular probe or circularizable probe or probe set comprising one or more modified nucleotide or nucleotide analog residues, wherein the circular probe or circularizable probe or probe set comprises a target hybridization region complementary to a target sequence of a target nucleic acid; and
(b) a polymerase;
wherein the one or more modified nucleotide or nucleotide analog residues decreases the polymerization rate of the polymerase in a rolling circle amplification reaction using the circular probe or a circularized probe generated from the circularizable probe or probe set as template as compared to a reference circular template without the one or more modified nucleotide or nucleotide analog residues.
   96. The kit of embodiment 95, further comprising a ligase for generating the circularized probe from the circularizable probe or probe set.
   97. The kit of embodiment 95 or 96, wherein the one or more modified nucleotide or nucleotide analog residues comprise modified sugar nucleotide residues and/or backbone modified nucleotide or nucleotide analog residues.
   98. The kit of any of embodiments 95-97, wherein the one or more modified nucleotide or nucleotide analog residues are selected from the group consisting of 2′-O-methyl ribonucleic acid (2′-OMeRNA), locked nucleic acid (LNA) nucleotides, 2′-fluoro ribonucleic acid (2′-F RNA), phosphorothioate backbone nucleotides, thiophosphate backbone nucleotides, triazole-modified nucleotides, and combinations thereof.
   99. The kit of any of embodiments 95-98, further comprising:
one or more non-incorporable nucleotides or analogs thereof configured to bind transiently to the polymerase but not be incorporated by the polymerase, and/or
one or more incorporable nucleotides or analogs thereof configured to be incorporated by the polymerase at a slower rate than a corresponding nucleoside triphosphate.
   100. A kit for performing rolling circle amplification (RCA), the kit comprising:
(a) a polymerase;
(b) one or more non-incorporable nucleotides or analogs thereof configured to bind transiently to the polymerase but not be incorporated by the polymerase, and/or one or more incorporable nucleotides or analogs thereof configured to be incorporated by the polymerase at a slower rate than a corresponding nucleoside triphosphate; and
(c) one or more circular probes to be used as a template for RCA, one or more circularizable probes or probe sets for generating a circularized probe to be used as a template for RCA, and/or one or more reagents for generating a circularized template for RCA.
   101. The kit of embodiment 99 or 100, wherein the one or more non-incorporable nucleotides or analogs thereof and/or one or more incorporable nucleotides or analogs thereof comprise an alpha-thiol nucleotide, an alkyne modified nucleotide, an azide modified nucleotide, a diphosphate nucleotide, and/or a monophosphate nucleotide.
   102. The kit of any of embodiments 95-101, further comprising unmodified deoxyribose nucleotide triphosphates.
   103. The kit of any of embodiments 100-102, wherein any combination of the one or more the one or more non-incorporable nucleotides or analogs thereof, the one or more incorporable nucleotides or analogs thereof, and/or the unmodified deoxyribose nucleotide triphosphates are comprised by a reaction mixture.
   104. A kit for performing rolling circle amplification, the kit comprising:
(a) a polymerase;
(b) one or more modified nucleotides or nucleotide analogs comprising one or more hydrophobic modifications configured to be incorporated by the polymerase; and
(c) one or more circular probes to be used as a template for rolling circle amplification (RCA), one or more circularizable probes or probe sets for generating a circularized probe to be used as a template for RCA, and/or one or more reagents for generating a circularized template for rolling circle amplification.
   105. The kit of embodiment 104, wherein the one or more modified nucleotides or nucleotide analogs comprise a modified dUTP, wherein each modified dUTP is independently selected from ethynyl-dUTP and vinyl dUTP.
   106. The kit of embodiment 104 or 105, wherein the one or more modified nucleotides or nucleotide analogs are provided in a reaction mixture comprising the one or more nucleotide analogs and unmodified deoxyribose nucleotide triphosphates.
   107. The kit of embodiment 105 or 106, wherein the ratio of the modified dUTP to an unmodified dTTP in the reaction mixture is at least about 80:20.

It should be appreciated that all combinations of the methods and systems described are contemplated as being part of the inventive subject matter disclosed herein. In particular, all combinations of claimed subject matter appearing in this disclosure are contemplated as being part of the inventive subject matter disclosed herein.

### EXAMPLES

The following examples are included for illustrative purposes only and are not intended to limit the scope of the present disclosure.

### Example 1: In Situ Analysis Using Probes Comprising Modified Nucleotide or Nucleotide Analog Residues for Reducing the Rate of Rolling Circle Amplification

This Example discloses exemplary methods for in situ target nucleic acid detection using rolling circle amplification (RCA). In some aspects, improved target detection and/or image analysis can be achieved by reducing the rate of polymerization of in situ RCA. In some examples, the methods disclosed herein facilitate providing RCA products having smaller intensities and/or smaller sizes.

In some examples, reducing the rate of polymerization of RCA (e.g., RCA performed in situ in a cell or tissue sample) can be achieved by using a circular probe or circularizable probe or probe set comprising one or more modified nucleotide or nucleotide analog residues.

A biological sample (e.g., a processed or cleared biological sample, a tissue sample, a sample embedded in a hydrogel, etc.) is contacted with a circular probe or circularizable probe or probe set (e.g., a padlock probe, SNAIL probe, RollFISH probe, PLAYR, probe, etc.). In separate experimental conditions, the circularizable probe or probe set (a) does not comprise modified nucleotide or nucleotide analog residues (e.g., as shown in FIG. 1B), (b) comprises one or more 2′-OMeRNA nucleotide residues (e.g., as shown in FIGS. 1A and 1C, comparing the structure of 2′-OMeRNA to an unmodified nucleotide residue), or (c) comprises one or more triazole-based linkages (e.g., as shown in FIG. 1D). In some examples, the circularizable probe or probe set comprises any combination of modified nucleotide or nucleotide analog residues. The modified nucleotide or nucleotide analog residues are capable of serving as a template for RCA but result in a reduced rate of polymerization. In an example to separately demonstrate different rates of polymerization of the RCA probe, the nucleotide sequence of the circularizable probe or probe set in (a), (b), and (c) can be identical with the exception of the modified nucleotide or nucleotide analog residues.

An RCA reaction is performed for each probe design under the same amplification conditions. Circularizable probes are hybridized to a target nucleic acid sequence, such as mRNA or cDNA, in the biological sample and are ligated to generate a circularized probe. An RCA primer is hybridized to the ligated (circularized) probe, and an RCA reaction mixture (containing Phi29 reaction buffer, dNTPs, and Phi29 polymerase) is added to the sample. The sample is incubated at an incubation temperature (e.g., 30° C. or 37° C.) for a defined period of time (e.g. 3 hours), allowing the closed circle to be used as a template for RCA, and amplified by the DNA polymerase to generate a RCA product. The RCA reaction is terminated by washing the sample with TE buffer.

RCA products in each condition are hybridized to fluorophore-labeled (e.g. Cy5-labeled) detectable probes at room temperature and imaged to assess RCA product size. Prior to imaging, tissue samples are treated with Gold Antifade Mounting medium to optimize the light path and prolong sample integrity. Imaging is carried out at 40× magnification using an Orca Fusion camera.

RCA products in conditions where the probe comprises 2′-OMeRNA nucleotide residues or triazole-based linkages are expected to be reduced in size (e.g. have a smaller mean diameter) in comparison to the conditions where the probe does not comprise modified nucleotide or nucleotide analog residues.

In another example, the biological sample can be contacted with a plurality of circularizable probes, wherein each circularizable probe hybridizes to a target nucleic acid in the sample. In one example, a first target nucleic acid (mRNA 1) is highly abundant (e.g., is present at a high density) in the sample, and a second target nucleic acid (mRNA 2) is less abundant (e.g., is present at a low density) in the sample. The sample can be contacted with a first circularizable probe comprising one or more modified nucleotide or nucleotide analog residues (e.g., comprising one or more 2′-OMeRNA and/or triazole linkages) that hybridizes to a target sequence comprised by mRNA 1, and a second circularizable probe that does not comprise modified nucleotide or nucleotide analog residues and that hybridizes to a target sequence comprised by mRNA 2. The circularizable probes can be ligated and RCA can be performed as described above to generate first and second RCA products in the sample. The rate of polymerization on the first circularized probe is less than the rate of polymerization on the second circularized probe, yielding a smaller first RCA product relative to the second RCA product (e.g., as depicted schematically in FIG. 1A and FIG. 1B).

### Example 2: Use of Nucleotides or Analogues Thereof in a Reaction Mixture for Reducing the Rate of Rolling Circle Amplification

This Example discloses exemplary methods that improve in situ target detection and image analysis using rolling circle amplification (RCA). Improved target detection and image analysis can be achieved by reducing the rate of polymerization of in situ RCA, thereby providing RCA products having smaller intensities and sizes. Reducing the rate of polymerization of in situ RCA can be achieved by including in the reaction one or more nucleotides or analogues thereof, which comprise (i) a non-incorporable nucleotide or analog thereof that binds transiently to the polymerase but is not incorporated by the polymerase, and/or (ii) an incorporable nucleotide or analog thereof that is configured to be incorporated by the polymerase at a slower rate than a corresponding nucleoside triphosphate.

This Example provides an exemplary method for analyzing a biological sample using RCA. In particular, this example provides an exemplary method for including nucleotides or analogues thereof in an RCA reaction mixture to reduce the rate of polymerization, and thus reduce the size of resulting RCA products.

In some examples, a biological sample (e.g., a processed or cleared biological sample, a tissue sample, a sample embedded in a hydrogel, etc.) is contacted with a circular probe or circularizable probe or probe set (e.g., a padlock probe). For example, circularizable probes are hybridized to a target nucleic acid sequence, such as mRNA or cDNA, in the biological sample and are ligated to generate a circularized probe from the circularizable probe. An RCA primer is hybridized to the circularized probe. In some examples, the circular probe or circularizable probe or probe set does not comprise any modified nucleotide or nucleotide analog residues. In other examples, circular or circularizable probes or probe sets comprising modified nucleotide or nucleotide analog residues that reduce the rate of polymerization by a polymerase on the template can be used (e.g., as described in Example 1).

In other examples, a circular template for RCA can be a circularized cDNA molecule (e.g., generated using CircLigase™, a thermostable ATP-dependent ligase that catalyzes intramolecular ligation (e.g., circularization) of ssDNA templates having a 5′-phosphate and a 3′-hydroxyl group). An RCA primer can similarly be hybridized to the circularized cDNA molecule.

An RCA reaction mixture containing Phi29 reaction buffer, Phi29 polymerase, and unmodified dNTPs (dATP, dTTP, dCTP, dGTP) is added to the sample. In separate experimental conditions, the reaction mixture further comprises (a) no additional nucleotides or analogues thereof, (b) alpha-thiol nucleotides, (c) diphosphate nucleotides, or (d) monophosphate nucleotides. In other examples, the reaction mixture can comprise any combination of alpha-thiol nucleotides, diphosphate nucleotides, and/or monophosphate nucleotides.

The sample is incubated at an incubation temperature (e.g., 30° C. or 37° C.) for a defined period of time (e.g. 3 hours), allowing the circularized probe to be used as a template for RCA and to be amplified by the DNA polymerase to generate a RCA product. The RCA reaction is terminated by washing the sample with TE buffer.

RCA products in each condition are hybridized to fluorophore-labeled (e.g. Cy5-labeled) detectable probes at room temperature and imaged to assess RCA product size. Prior to imaging, tissue samples can be treated with Gold Antifade Mounting medium to optimize the light path and prolong sample integrity. Imaging can be carried out at 40× magnification, with a 4×4 field-of-view (FOV) for each section, using an Orca Fusion camera.

RCA products in conditions where alpha-thiol nucleotides, diphosphate nucleotides, or monophosphate nucleotides are included in the reaction mixture are expected to be reduced in size in comparison to the condition where only unmodified dNTPs are included.

### Example 3: Use of Modified Nucleotides in a Reaction Mixture for Reducing the Rate of Rolling Circle Amplification In Situ

Example 2 above describes exemplary methods for reducing the rate of rolling circle amplification (RCA) using various incorporable nucleotides or analogues thereof that are incorporated by the polymerase at a slower rate than corresponding unmodified nucleoside triphosphates. This Example demonstrates a method for analyzing a biological sample using rolling circle amplification (RCA), wherein exemplary incorporable nucleotides 5-Azido-PEG₄-dCTP and 5-Ethynyl-dUTP were included in an RCA reaction mixture to reduce the rate of polymerization. The reduced rate of polymerization results in RCA products of smaller size and intensity.

Sections of fresh/frozen mouse brains were prepared for in situ analysis by formalin fixation and permeabilization.

Padlock probes targeting the genes Prox1 and Satb2 were added to the sample and allowed to hybridize overnight at 37° C. Following probe hybridization, samples were washed in formamide and SSC to remove unbound probes. For padlock probe ligation, samples were incubated with 1.25 U/μL SplintR Ligase in SplintR ligase buffer for 2 hours at 37° C. Primers for rolling circle amplification (RCA) were added to the sample and allowed to hybridize to the circularized padlock probes for 30 minutes at 30° C.

RCA reaction mixture containing Phi29 polymerase, Phi29 reaction buffer, and dNTPs was added to each sample. In separate conditions, RCA was allowed to proceed for 2 hours or 4 hours. Control conditions included unmodified dNTPs only (dATP, dGTP, dCTP, dTTP), and experimental conditions included the modified dNTPs 5-Azido-PEG₄-dCTP and 5-Ethynyl-dUTP. For the experimental conditions, each of the modified nucleotides were substituted for 75% of the unmodified corresponding nucleotide (100% of dATP nucleotides were unmodified, 100% of dGTP nucleotides were unmodified, 25% of the dCTP nucleotides were unmodified dCTP and 75% were 5-Azido-PEG₄-dCTP, and 25% of the dTTP nucleotides were unmodified and 75% were 5-Ethynyl-dUTP).

Experimental conditions were set up as shown in Table 1 below.

TABLE 1

| Condition | RCA Time | Modified dNTPs present |
|---|---|---|
| 1 | 2 hours | No |
| 2 | 2 hours | Yes (75%) |
| 3 | 4 hours | No |
| 4 | 4 hours | Yes (75%) |

RCA reactions were terminated, and intermediate probes (e.g., L-shaped probes) corresponding to Prox1 or Satb2 were hybridized to corresponding RCA products (RCPs). Each intermediate probe comprised a region hybridizing to a target sequence of the corresponding RCA product and an overhang for binding fluorescently labeled probes (e.g., detection oligos, DOs). Fluorescently labeled probes were then hybridized to the intermediate probes. Samples were imaged by fluorescence microscopy to assess the density, size, and intensity of detectable signals associated with the RCPs in the dentate gyrus and cortex.

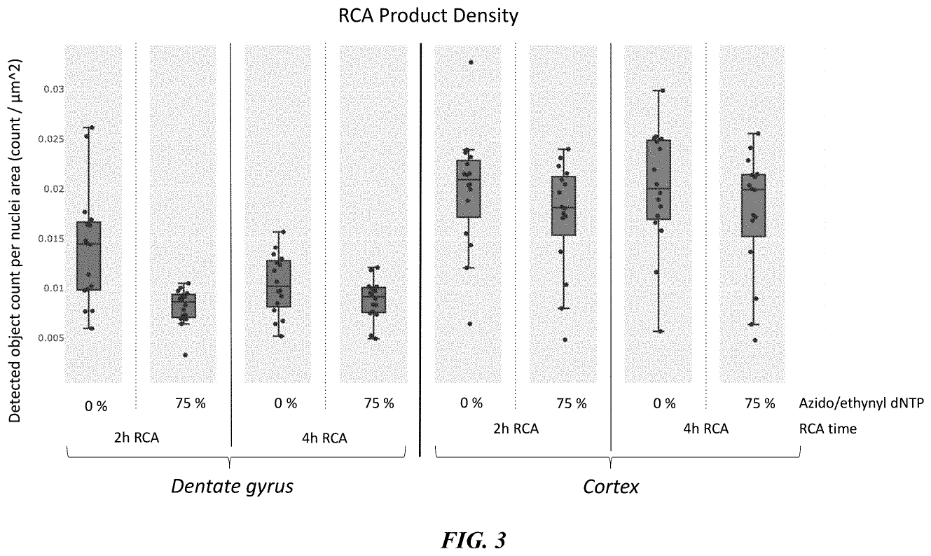

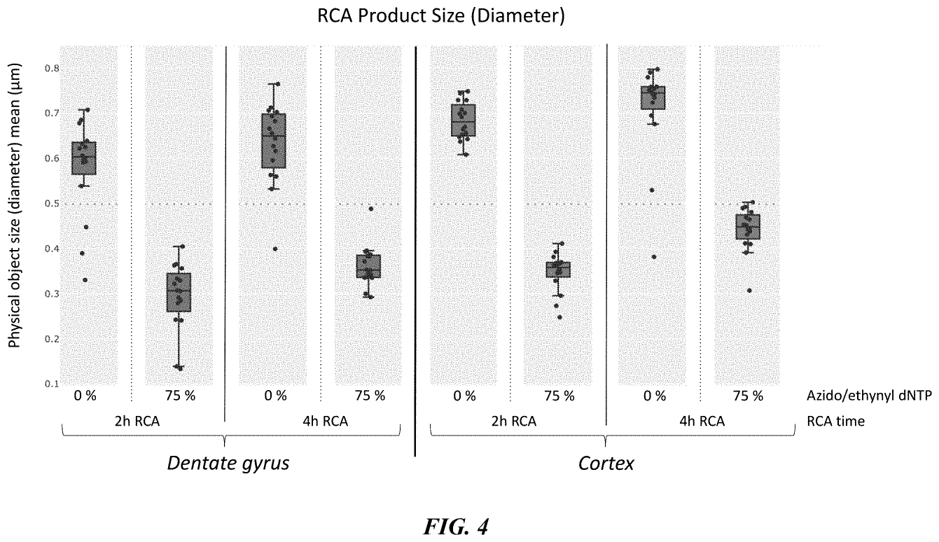

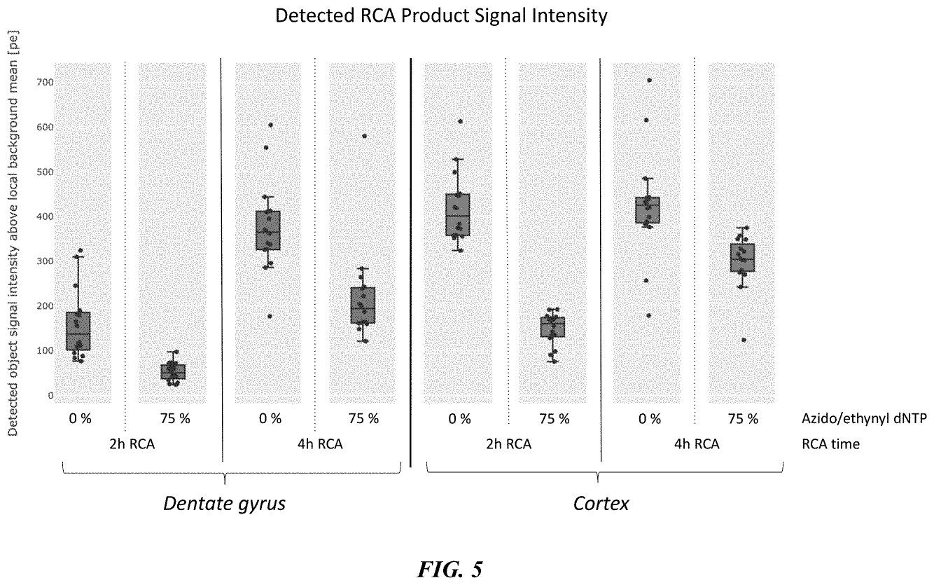

As shown in FIG. 3, the density of RCPs was not significantly affected by the presence of modified dNTPs. As shown in FIG. 4, however, RCP size was decreased in conditions where modified nucleotides were present in the RCA reaction, both for 2 hour and 4 hour conditions. As shown in FIG. 5, RCP intensity was decreased in conditions where modified nucleotides were present in the RCA reaction, both for 2 hour and 4 hour conditions.

The results demonstrate that inclusion of modified dNTPs can reduce the rate of RCA, resulting in reduction in the size of RCPs, and/or the size and intensity of detectable signals associated with RCPs.

### Example 4: Quantitative RCA Demonstrates Decreased Polymerization Rate Using Modified Nucleotides

Example 3 above demonstrates that in situ RCA using modified incorporable nucleotides can decrease the size and/or intensity of the resulting RCA products. This Example provides quantitative RCA results demonstrating a decreased rate of polymerization in a reaction mixture comprising the modified dNTPs 5-Azido-PEG₄-dCTP and 5-Ethynyl-dUTP in an RCA reaction mixture to reduce the rate of polymerization.

Quantitative RCA (qRCA) was performed to determine the effect of specific modified dNTPs on the rate of RCA. Reactions included Phi29 polymerase, Phi29 reaction buffer, circular RCA template oligonucleotides, RCA primers, and dNTPs including 0%, 50%, or 100% of modified nucleotides, as shown in Table 2. As an example, 50% 5-Azido-PEG₄-dCTP indicates that: 50% of the dCTP nucleotides are unmodified dCTP and 50% are 5-Azido-PEG₄-dCTP. Negative controls included conditions with no circular oligonucleotides, no dNTPs, or no Phi29 polymerase. Reactions also included SYBR Gold to facilitate quantification of the rate of polymerization. Reactions were incubated at 37° C. and imaged every 30 seconds for 1 hour.

TABLE 2

| Condition | Modified dNTPs | % modified dNTPs |
|---|---|---|
| 1 | 5-Azido-PEG₄-dCTP/ | 0% |
| | 5-Ethynyl-dUTP (5-EdUTP) | |
| 2 | 5-Azido-PEG₄-dCTP/ | 50% |
| | 5-Ethynyl-dUTP (5-EdUTP) | |
| 3 | 5-Azido-PEG₄-dCTP/ | 100% |
| | 5-Ethynyl-dUTP (5-EdUTP) | |
| 4 | 5-Azido-PEG₄-dCTP | 0% |
| 5 | 5-Azido-PEG₄-dCTP | 50% |
| 6 | 5-Azido-PEG₄-dCTP | 100% |
| 7 | 5-Ethynyl-dUTP (5-EdUTP) | 0% |
| 8 | 5-Ethynyl-dUTP (5-EdUTP) | 50% |
| 9 | 5-Ethynyl-dUTP (5-EdUTP) | 100% |

As shown in FIG. 6B, inclusion of 5-Azido-PEG₄-dCTP and 5-Ethynyl-dUTP, alone or in combination, reduced the rate of RCA in a concentration-dependent manner. The effect on the rate of RCA was additive, with the largest reduction in the rate of RCA observed for the reaction incorporating both 5-Azido-PEG₄-dCTP and 5-Ethynyl-dUTP (FIG. 6B, left panel). A stronger effect for an individual modified nucleotide was observed for 5-Azido-PEG₄-dCTP (FIG. 6B, middle panel) compared to 5-Ethynyl-dUTP (FIG. 6B, right panel). As 5-Azido-PEG₄-dCTP is the bulkier of the two modifications (FIG. 6A), these results suggest that the decrease in polymerization rate may be correlated with the size of the base modification.

The results above demonstrate that modified dNTPs or analogues thereof can decrease the rate of RCA reactions in a concentration and modification-dependent manner. The results therefore support the use of incorporable nucleotides or analogues thereof configured to be incorporated by the polymerase at a slower rate than a dNTP (e.g., dATP, dTTP/dUTP, dCTP, or dGTP) to control the rate of RCA reactions.

### Example 5: Use of Modified Nucleotides in a Reaction Mixture for Reducing the Size of RCA Products In Situ

This Example discloses exemplary methods that improve in situ target detection and image analysis using rolling circle amplification (RCA). Improved target detection and image analysis can be achieved by reducing the size of RCA products. Reducing RCA product size can be achieved by including in the reaction one or more nucleotides or analogues thereof, for example that comprise hydrophobic modifications, such as 5-Ethynyl-dUTP (5-EdUTP) and/or 5-Vinyl-dUTP.

The Examples above describe exemplary methods for reducing the rate of RCA. This Example provides an exemplary method for analyzing a biological sample using RCA, wherein exemplary nucleotides with added hydrophobic groups (5-Ethynyl-dUTP (5-EdUTP) or 5-Vinyl-dUTP) were included in an RCA reaction mixture to promote compaction of RCA products. The inclusion of the modified nucleotides resulted in smaller RCA product size.

Sections of fresh/frozen mouse brains were prepared for in situ analysis by formalin fixation and permeabilization. Padlock probes targeting the genes Prox1 and Satb2 were added to the sample and allowed to hybridize overnight at 37° C. Following probe hybridization, samples were washed in formamide and SSC to remove unbound probes. For padlock probe ligation, samples were incubated with SplintR Ligase and an RNase inhibitor in ligase buffer for 2 hours at 37° C. Primers for rolling circle amplification (RCA) were added to the sample and allowed to hybridize to the circularized padlock probes for 30 minutes at 37° C.

An RCA reaction mixture with Phi29 polymerase and 100 μM dNTPs was added to each sample (100 μM of dATP, 100 μM of dCTP, 100 μM of dGTP, and 100 μM of combined dTTP and modified dUTP). The modified dUTPs 5-Ethynyl-dUTP (5-EdUTP) or 5-Vinyl-dUTP were added to the reaction mixture at the following concentrations: 0 μM (control), 1 μM, 1.25 μM, 2.5 μM, 5 μM, 10 μM, 20 μM, 40 μM, 80 μM, or 100 μM, wherein the modified dUTP replaced unmodified dTTP in the reaction mixture for a combined total concentration of 100 μM modified dUTP and dTTP. Structural formulas of 5-EdUTP and 5-Vinyl-dUTP are shown in FIG. 7 (with hydrophobic modifications shown in dashed circles).

RCA was allowed to proceed for 2 hours at 37° C. RCA reactions were terminated, and intermediate probes corresponding to Prox1 or Satb2 were hybridized to corresponding RCA products (RCPs). Each intermediate probe comprised a region hybridizing to a target sequence of the corresponding RCA product and an overhang for binding fluorescently labeled probes. Fluorescently labeled (Cy3) probes were then hybridized to the intermediate probes. Samples were imaged by fluorescence microscopy to assess the density, size, and intensity of detectable signals associated with the RCPs.

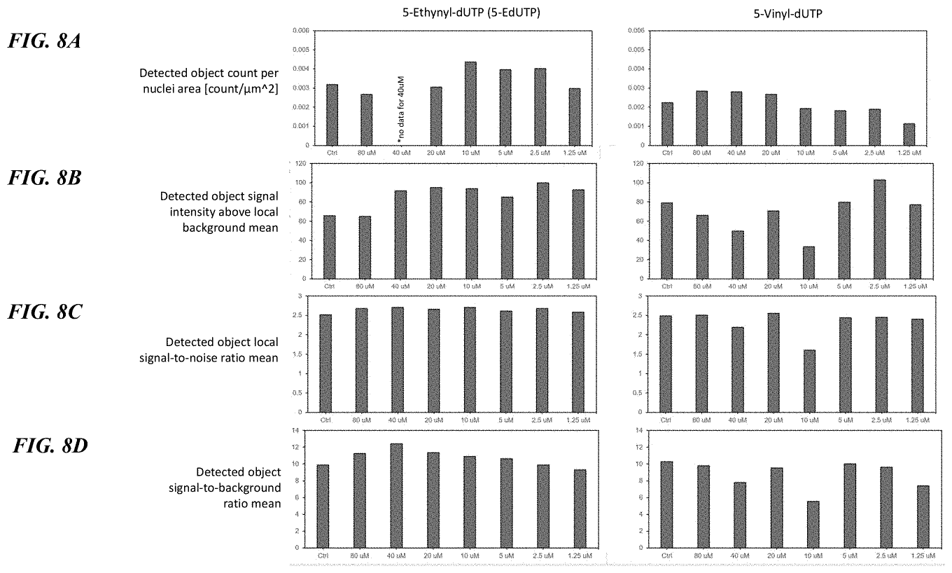

As shown in FIG. 8A, the density of RCPs was not significantly affected by the presence of modified dNTPs. Signal intensity above local background (FIG. 8B), local signal to noise ratio (FIG. 8C), and signal to background ratio (FIG. 8D) were also not affected by the presence of the modified dNTPs.

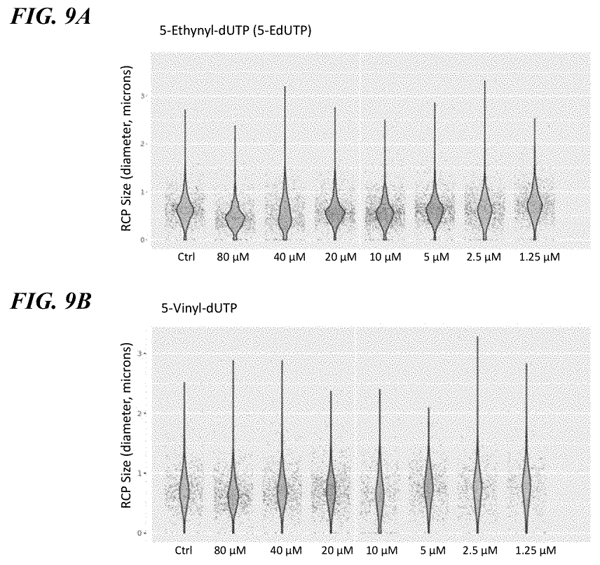

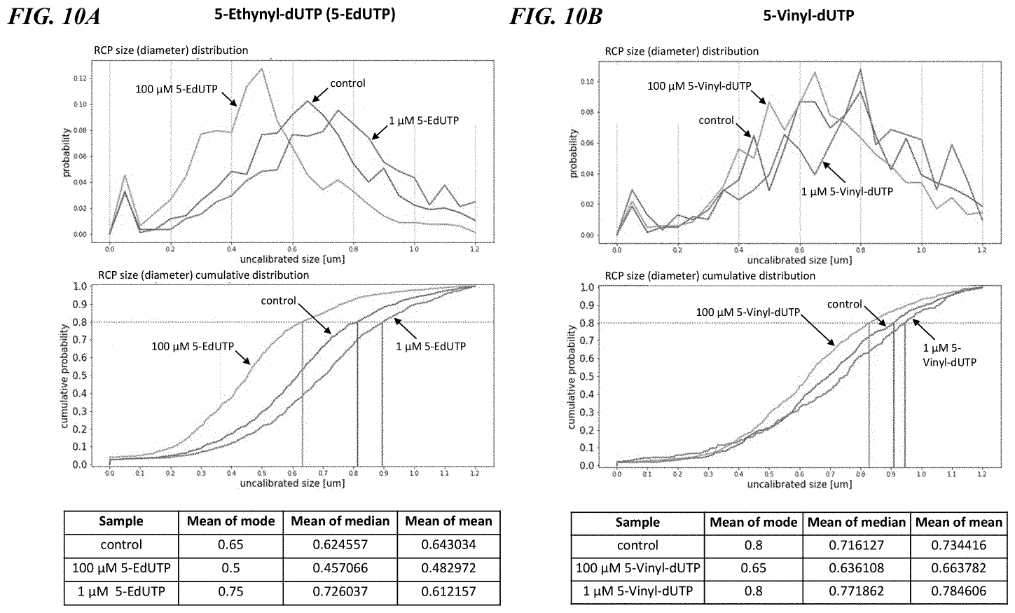

As shown in FIGS. 9A-9B, the distribution of RCP size (e.g., as measured by diameter) was decreased with inclusion of 5-EdUTP or 5-Vinyl-dUTP, with a marked reduction of RCP size with inclusion of 80 μM 5-EdUTP. As shown in FIGS. 10A-10B for a different set of concentrations, the distribution of RCP size was decreased with inclusion of 5-EdUTP or 5-Vinyl-dUTP, with a marked reduction of RCP size distribution with inclusion of 100 μM 5-EdUTP.

The results demonstrate that inclusion of nucleotide analogs comprising hydrophobic modifications can reduce RCP size, and/or the size of detectable signals associated with RCPs.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1. A method for analyzing a biological sample, comprising:
(a) contacting the biological sample with a reaction mixture comprising one or more modified nucleotides or nucleotide analogs comprising a modified nucleotide or nucleotide analog having a hydrophobic modification,
(b) performing rolling circle amplification (RCA) of a circular nucleic acid template in the biological sample using a polymerase, thereby generating an RCA product incorporating the one or more modified nucleotides or nucleotide analogs, wherein the RCA product is not crosslinked to another molecule via the one or more modified nucleotides or nucleotide analogs incorporated into the RCA product, and
(c) detecting the RCA product not crosslinked via the one or more modified nucleotides or nucleotide analogs at a location in the biological sample.

2. The method of claim 1, wherein the hydrophobic modification is a base modification.

3. The method of claim 1, wherein the hydrophobic modification comprises a carbon chain and/or a hydrocarbon ring.

4. The method of claim 1, wherein the hydrophobic modification comprises a triple bond.

5. The method of claim 1, wherein the hydrophobic modification comprises a vinyl or ethynyl group.

6. The method of claim 1, wherein the modified nucleotide or nucleotide analog having the hydrophobic modification is an ethynyl-dUTP or a vinyl-dUTP.

7. The method of claim 6, wherein the modified nucleotide or nucleotide analog having the hydrophobic modification is a 5-ethynyl-dUTP or a 5-vinyl-dUTP.

8-11. (canceled)

12. The method of claim 1, wherein the median diameter of an RCA product generated using the one or more modified nucleotides or nucleotide analogs is smaller than the median diameter of a reference RCA product produced using the same template without including the one or more modified nucleotides or nucleotide analogs in the reaction mixture.

13. (canceled)

14. The method of claim 12, wherein the median diameter of an RCA product generated using the one or more modified nucleotides or nucleotide analogs is no more than 90% or no more than 80% of the median diameter of a reference RCA product produced using the same template without including the one or more modified nucleotides or nucleotide analogs in the reaction mixture.

15-17. (canceled)

18. The method of claim 1, wherein:
the one or more modified nucleotides or nucleotide analogs incorporated into the RCA product do not comprise an amine; and/or
the one or more modified nucleotides or nucleotide analogs incorporated into the RCA product do not comprise a fluorophore.

19. The method of claim 1, comprising in (c), contacting the biological sample with a detectably labeled nucleic acid probe that directly or indirectly binds to the RCA product.

20. (canceled)

21. A method for analyzing a biological sample, comprising:
(a) contacting the biological sample with a reaction mixture comprising one or more nucleotides or nucleotide analogs,
(b) performing rolling circle amplification (RCA) of a circular nucleic acid template in the biological sample using a polymerase, thereby generating an RCA product,
wherein the one or more nucleotides or nucleotide analogs comprise:
(i) a non-incorporable nucleotide or analog thereof that is not incorporated by the polymerase, and/or
(ii) an incorporable nucleotide or nucleotide analog that is configured to be incorporated by the polymerase at a slower rate than a corresponding nucleoside triphosphate, and
(c) detecting the RCA product which is not crosslinked via nucleotide(s) or nucleotide analog(s) at a location in the biological sample.

22. The method of claim 21, wherein in (c), the RCA product is not crosslinked, via a nucleotide or nucleotide analog incorporated into the RCA product, to the RCA product itself, to another molecule in the biological sample, or to a matrix embedding the biological sample.

23. The method of claim 21, wherein:
the one or more nucleotides or nucleotide analogs incorporated into the RCA product do not comprise an amine; and/or
the one or more nucleotides or nucleotide analogs incorporated into the RCA product do not comprise a fluorophore.

24. The method of claim 21, comprising in (c), contacting the biological sample with a detectably labeled nucleic acid probe that directly or indirectly binds to the RCA product.

25. The method of claim 21, wherein the presence of the one or more modified nucleotides or nucleotide analogs in the reaction mixture decreases the polymerization rate of the polymerase and/or the size of the RCA product as compared to a reference reaction mixture without the one or more modified nucleotides or nucleotide analogs.

26. (canceled)

27. A method for analyzing a biological sample, comprising:
(a) contacting the biological sample with a circular probe or circularizable probe or probe set comprising one or more modified nucleotide or nucleotide analog residues, wherein the circular probe or circularizable probe or probe set comprises a hybridization region that hybridizes to a target nucleic acid in the biological sample, and wherein the one or more modified nucleotide or nucleotide analog residues are outside the hybridization region,
(b) using a polymerase to perform rolling circle amplification (RCA) of the circular probe or of a circularized probe generated from the circularizable probe or probe set, thereby generating an RCA product,
wherein the presence of the one or more modified nucleotide or nucleotide analog residues decreases the polymerization rate of the polymerase on the circular or circularized probe and/or the size of the RCA product as compared to a reference circular template without the one or more modified nucleotide or nucleotide analog residues, and
(c) detecting the RCA product at a location in the biological sample.

28. The method of claim 27, wherein the one or more modified nucleotide or nucleotide analog residues comprise modified deoxyribonucleotide (DNA) or DNA analog residues and/or modified ribonucleotide (RNA) or RNA analog residues.

29. The method of claim 27, wherein the one or more modified nucleotide or nucleotide analog residues do not comprise an amine and/or a fluorophore.

30. (canceled)

31. The method of claim 1, wherein the biological sample is a non-homogenized tissue sample or a tissue section.
